(19) United States
(12) Patent Application Publication
(10) Pub. No.: US 2023/0357193 A1
(43) Pub. Date: Nov. 9, 2023

(54) COMPOUND CAPABLE OF INHIBITING AND DEGRADING ANDROGEN RECEPTORS, AND PHARMACEUTICAL COMPOSITIONS AND PHARMACEUTICAL USES THEREOF

(71) Applicant: Sichuan Haisco Pharmaceutical Co., Ltd., Chengdu City (CN)

(72) Inventors: Chen ZHANG, Chengdu City (CN); Yuting LIAO, Chengdu City (CN); Guozhi ZHU, Chengdu City (CN); Fei YE, Chengdu City (CN); Xinfan CHENG, Chengdu City (CN); Xiaogang CHEN, Chengdu City (CN); Pingming TANG, Chengdu City (CN); Yao LI, Chengdu City (CN); Jia NI, Chengdu City (CN); Pangke YAN, Chengdu City (CN)

(21) Appl. No.: 18/004,708

(22) PCT Filed: Jul. 8, 2021
(86) PCT No.: PCT/CN2021/105275

§ 371 Date: Jan. 9, 2023

(30) Foreign Application Priority Data
Jul. 9, 2020 (CN) 202010655931.0
Aug. 7, 2020 (CN) 202010776540.4

(51) Int. Cl. C07D 401/14; C07D 417/14; C07D 471/04; C07D 471/08; C07D 471/10

(52) U.S. Cl. CPC C07D 401/14; C07D 417/14; C07D 471/04; C07D 471/08; C07D 471/10

(57) ABSTRACT

A compound as shown in general formula B-L-K (I) or stereoisomers, deuterated compounds, solvates, prodrugs, metabolites, pharmaceutically acceptable salts or co-crystals thereof, and intermediates thereof and uses thereof for AR-related diseases such as prostate cancer.

### TECHNICAL FIELD

The present disclosure relates to a compound as shown in general formula (I) or a stereoisomer, a deuterated compound, a solvate, a prodrug, a metabolite, a pharmaceutically acceptable salt or a co-crystal thereof, and intermediates thereof and preparation methods therefor, as well as the use thereof in AR-related diseases such as prostate cancer.

### BACKGROUND ART

Prostate cancer is mostly found in early stages, and its pathogenesis is often related to genetic factors, high-fat diet and endocrine. Generally, prostate cancer is more prevalent in developed countries than in developing countries. In 2016, there were 120,000 new prostate cancer patients in China, and by 2030, it is expected that the number of new patients will reach 237,000, and the market share will reach 4.8 billion US dollars. Patients with early-stage prostate cancer may be treated with radical therapy and have a longer survival time, while advanced patients with metastasis may be treated by a castration procedure combined with anti-androgen therapy and may develop castration-resistant prostate cancer. Clinical studies have shown that in most patients with castration-resistant prostate cancer, androgen receptors (ARs) are overexpressed, and inhibition of androgen receptor (AR) signaling has significant efficacy in patients with hormone-refractory prostate cancer, so that inhibition of androgen receptors (ARs) is an effective means to directly block this pathway.

The androgen receptor (AR), a nuclear hormone receptor that is structurally comprised of an N-terminal domain (NTD), a DNA-binding domain (DBD) and a ligand-binding domain (LBD), can regulate the expression of genes that induce prostate cancer, and thus, inhibition of the androgen receptor is an effective method for treating prostate cancer. Currently marketed androgen receptor inhibitors such as enzalutamide and bicalutamide mainly exert an inhibitory effect by interacting with the ligand-binding domain (LBD) of the androgen receptor. However, some patients will develop drug resistance caused by androgen receptor splice variants (AR-Vs) with the deletion of LBD fragment during treatment. Preclinical studies have shown that androgen receptor splice variants can accelerate the progression of enzalutamide-resistant prostate cancer, and therefore, how to solve the problem of drug resistance has become the focus of clinical medicine.

PROTAC (proteolysis targeting chimera) molecules are a class of dual function compounds which are capable of binding to both targeted proteins and E3 ubiquitin ligases. Such compounds can be recognized by proteasomes in a cell to cause the degradation of the targeted proteins, which can effectively reduce the contents of the targeted proteins in the cell. By introducing a ligand capable of binding to various targeted proteins into the PROTAC molecules, it is possible to apply the PROTAC technology to the treatment of various diseases, and this technology has attracted extensive attention in recent years.

Therefore, it is necessary to develop a novel androgen receptor (AR) or/and AR splice variant inhibitor and a PROTAC drug of E3 ubiquitin ligase for the treatment of androgen receptor-related tumor diseases.

### SUMMARY OF THE DISCLOSURE

The present disclosure develops an AR or/and AR splice variant inhibitor with a novel structure, good efficacy, high bioavailability and higher safety, for use in the treatment of AR-related diseases such as prostate cancer.

The present disclosure provides a compound or a stereoisomer, a deuterated compound, a solvate, a prodrug, a metabolite, a pharmaceutically acceptable salt or a co-crystal thereof, wherein the compound is selected from a compound as shown in general formula (I),

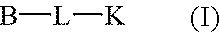

in certain embodiments, B is selected from

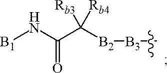

B₁ is selected from one of the following substituted or unsubstituted groups: 6-membered aryl or 6-membered heteroaryl, which, when substituted, is optionally further substituted with 0 to 4 R^{b1}, wherein the heteroaryl contains 1 to 4 heteroatoms selected from O, S and N;
B₂ is selected from one of the following substituted or unsubstituted groups: a 5-to 10-membered heterocyclic group or -NHC(=O)-, which, when substituted, is optionally further substituted with 0 to 4 R^{b2}, wherein the heterocyclic group contains 1 to 4 heteroatoms selected from O, S and N;
B₃ is selected from substituted or unsubstituted 5- to 6-membered aryl or a bond, wherein the 5- to 6-membered aryl, when substituted, is optionally further substituted with 0 to 4 R^{b2};
R^{b1} and R^{b2} are each independently selected from H, F, Cl, Br, I, OH, NH₂, CN, CF₃, —C(═O)NH₂, —C(═O)NH—C₁₋₄ alkyl, —C(═O)N(C₁₋₄ alkyl)₂, C₁₋₄ alkyl or C₁₋₄ alkoxy, wherein the alkyl or alkoxy is optionally further substituted with 0 to 4 substituents selected from H, F, Cl, Br, I or OH;
R^{b3} and R^{b4} are each independently selected from H or C₁₋₆ alkyl, wherein the alkyl is optionally further substituted with 0 to 4 substituents selected from H, F, Cl, Br, I, OH, NH₂, C₁₋₄ alkyl or C₁₋₄ alkoxy;
or R^{b3} and R^{b4} together with the carbon atoms to which they are attached form C₃₋₆ cycloalkyl or a C₃₋₆ mono-heterocyclic ring, wherein the cycloalkyl or mono-heterocyclic ring is optionally further substituted with 0 to 4 substituents selected from H, F, Cl, Br, I, OH, NH₂, C₁₋₄ alkyl or C₁₋₄ alkoxy, and the mono-heterocyclic ring contains 1 to 4 heteroatoms selected from O, S and N;
in certain embodiments, B is selected from

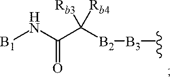

B₁ is selected from one of the following substituted or unsubstituted groups: 6-membered aryl or 6-membered heteroaryl, which, when substituted, is optionally further substituted with 0 to 4 R^{b1}, wherein the heteroaryl contains 1 to 4 heteroatoms selected from O, S and N;
B₂ is selected from one of the following substituted or unsubstituted groups: a 5-to 10-membered heterocyclic group or —NHC(═O)—, which, when substituted, is optionally further substituted with 0 to 4 R^{b2}, wherein the heterocyclic group contains 1 to 4 heteroatoms selected from O, S and N;
B₃ is selected from substituted or unsubstituted phenyl or a bond, wherein the phenyl, when substituted, is optionally further substituted with 0 to 4 R^{b2};
R^{b1} and R^{b2} are each independently selected from H, F, Cl, Br, I, OH, NH₂, CN, CF₃, —C(═O)NH₂, —C(═O)NH—CH₃, —C(═O)N(CH₃)₂, methyl, ethyl, propyl, isopropyl, methoxy or ethoxy, wherein the methyl, ethyl, propyl, isopropyl, methoxy or ethoxy is optionally further substituted with 0 to 4 substituents selected from H, F, Cl, Br, I or OH;
R^{b3} and R^{b4} are each independently selected from H or C₁₋₃ alkyl, wherein the alkyl is optionally further substituted with 0 to 4 substituents selected from H, F, Cl, Br, I, OH, NH₂, C₁₋₄ alkyl or C₁₋₄ alkoxy;
or R^{b3} and R^{b4} together with the carbon atoms to which they are attached form C₃₋₆ cycloalkyl or a C₃₋₆ mono-heterocyclic ring, wherein the cycloalkyl or mono-heterocyclic ring is optionally further substituted with 0 to 4 substituents selected from H, F, Cl, Br, I, OH, NH₂, C₁₋₄ alkyl or C₁₋₄ alkoxy, and the mono-heterocyclic ring contains 1 to 4 heteroatoms selected from O, S and N;
in certain embodiments, B is selected from

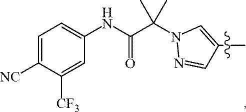

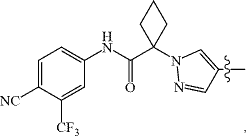

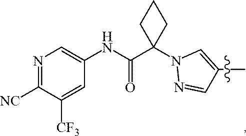

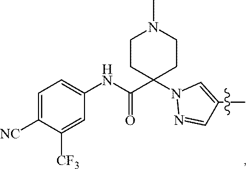

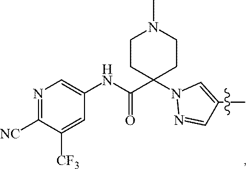

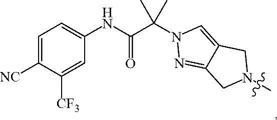

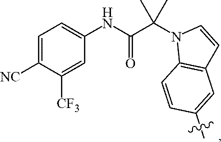

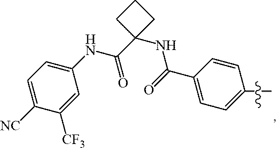

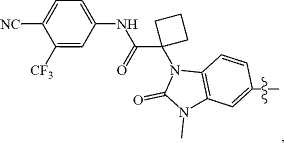

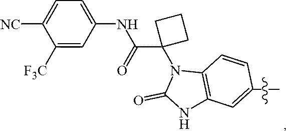

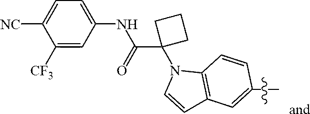

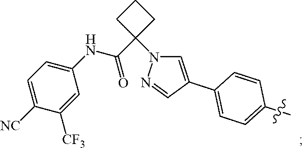

in certain embodiments, L is selected from -Ak1-Cy1-Ak2-Cy2-Ak3-Cy3-Ak4-Cy4-Ak5-;
Ak1, Ak2, Ak3, Ak4 and Ak5 are each independently selected from CH₂, O, C≡C or a bond;
Cy1, Cy2, Cy3 and Cy4 are each independently selected from a bond, a 4- to 7-membered mono-heterocyclic ring, a 5- to 10-membered fused heterocyclic ring, a 6- to 12-membered spiro-heterocyclic ring, a 7- to 10-membered bridged-heterocyclic ring, 4- to 7-membered monocycloalkyl, 5- to 10-membered fused cycloalkyl, 6- to 12-membered spiro cycloalkyl, 7- to 10-membered bridged cycloalkyl, 5- to 10-membered heteroaryl or 6- to 10-membered aryl, wherein the aryl, heteroaryl, cycloalkyl, mono-heterocyclic ring, fused heterocyclic ring, spiro-heterocyclic ring or bridged-heterocyclic ring is optionally further substituted with 0 to 4 substituents selected from H, F, Cl, Br, I, OH, COOH, CN, NH₂, oxo, C₁₋₄ alkyl, halogen-substituted C₁₋₄ alkyl, hydroxyl-substituted C₁₋₄ alkyl or C₁₋₄ alkoxy, and the heteroaryl, mono-heterocyclic ring, fused heterocyclic ring, spiro-heterocyclic ring or bridged-heterocyclic ring contains 1 to 4 heteroatoms selected from O, S and N;
in certain embodiments, L is selected from -Ak1-Cy1-Ak2-Cy2-Ak3-Cy3-Ak4-Cy4-Ak5-A;
Ak1, Ak2, Ak3, Ak4 and Ak5 are each independently selected from CH₂, O, C≡C or a bond;
Cy1, Cy2, Cy3 and Cy4 are each independently selected from a bond, a 4- to 7-membered nitrogen-containing mono-heterocyclic ring, a 5- to 10-membered nitrogen-containing fused heterocyclic ring, a 7- to 10-membered bridged-heterocyclic ring, or a 6- to 12-membered nitrogen-containing spiro-heterocyclic ring, wherein the mono-heterocyclic ring, fused heterocyclic ring, bridged-heterocyclic ring or spiro-heterocyclic ring is optionally further substituted with 0 to 4 substituents selected from H, F, Cl, Br, I, OH, COOH, CN, NH₂, oxo, C₁₋₄ alkyl, halogen-substituted C₁₋₄ alkyl, hydroxyl-substituted C₁₋₄ alkyl or C₁₋₄ alkoxy, and the mono-heterocyclic ring, fused heterocyclic ring, bridged-heterocyclic ring or spiro-heterocyclic ring contains 1 to 4 heteroatoms selected from O, S and N; and
in certain embodiments, L is selected from -Ak1-Cy1-Ak2-Cy2-Ak3-Cy3-Ak4-Cy4-Ak5-;
Ak1, Ak2, Ak3, Ak4 and Ak5 are each independently selected from CH₂, O, C≡C or a bond;
Cy1, Cy2, Cy3 and Cy4 are each independently selected from a bond or one of the following substituted or unsubstituted groups: azetidinyl, azacyclopentyl, azacyclohexyl, piperidine, morpholine, piperazine, cyclopropyl-fused-azetidinyl, cyclopropyl-fused-azacyclopentyl, cyclopropyl-fused-azacyclohexyl, cyclopropyl-fused-piperidine, cyclobutyl-fused-azetidinyl, cyclobutyl-fused-azacyclopentyl, cyclobutyl-fused-azacyclohexyl, cyclobutyl-fused-piperidine, cyclopentyl-fused-azetidinyl, cyclopentyl-fused-azacyclopentyl, cyclopentyl-fused-azacyclohexyl, cyclopentyl-fused-piperidine, cyclohexyl-fused-azetidinyl, cyclohexyl-fused-azacyclopentyl, cyclohexyl-fused-azacyclohexyl, cyclohexyl-fused-piperidine, azetidinyl-fused-azetidinyl, azetidinyl-fused-azacyclopentyl, azetidinyl-fused-azacyclohexyl, azetidinyl-fused-piperidine, azacyclopentyl-fused-azetidinyl, azacyclopentyl-fused-azacyclopentyl, azacyclopentyl-fused-azacyclohexyl, azacyclopentyl-fused-piperidine, azacyclohexyl-fused-azetidinyl, azacyclohexyl-fused-azacyclopentyl, azacyclohexyl-fused-azacyclohexyl, azacyclohexyl-fused-piperidine, cyclobutyl-spiro-azetidinyl, cyclobutyl-spiro-azacyclopentyl, cyclobutyl-spiro-azacyclohexyl, cyclopentyl-spiro-azetidinyl, cyclopentyl-spiro-azacyclopentyl, cyclopentyl-spiro-azacyclohexyl, cyclohexyl-spiro-azetidinyl, cyclohexyl-spiro-azacyclopentyl, cyclohexyl-spiro-azacyclohexyl, azetidinyl-spiro-azetidinyl, azetidinyl-spiro-azacyclopentyl, azetidinyl-spiro-azacyclohexyl, azacyclopentyl-spiro-azetidinyl, azacyclopentyl-spiro-azacyclopentyl, azacyclopentyl-spiro-azacyclohexyl, azacyclohexyl-spiro-azetidinyl, azacyclohexyl-spiro-azacyclopentyl, azacyclohexyl-spiro-azacyclohexyl, cyclobutyl-spiro-piperidine, cyclopentyl-spiro-piperidine, cyclohexyl- spiro-piperidine, azetidinyl- spiro-piperidine, azacyclopentyl- spiro-piperidine, azacyclohexyl-spiro-piperidine,

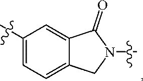

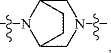

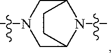

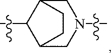

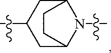

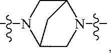

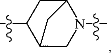

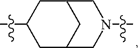

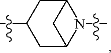

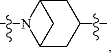

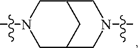

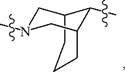

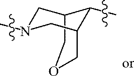

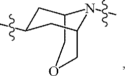

which, when substituted, is optionally further substituted with 0 to 4 substituents selected from H, F, Cl, Br, I, OH, NH₂, COOH, CN, oxo, C₁₋₄ alkyl, halogen-substituted C₁₋₄ alkyl, hydroxyl-substituted C₁₋₄ alkyl or C₁₋₄ alkoxy;
in certain embodiments, L is selected from -Ak1-Cy1-Ak2-Cy2-Ak3-Cy3-Ak4-Cy4-Ak5-;
Ak1, Ak2, Ak3, Ak4 and Ak5 are each independently selected from CH₂, O, C≡C or a bond;
Cy1, Cy2, Cy3 and Cy4 are each independently selected from one of the following substituted or unsubstituted groups: a bond,

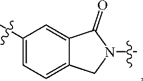

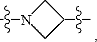

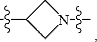

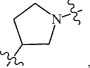

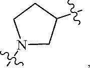

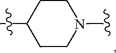

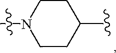

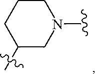

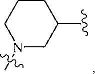

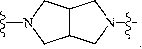

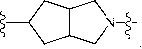

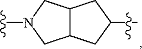

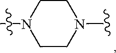

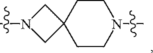

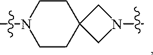

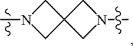

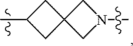

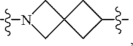

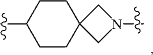

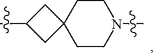

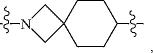

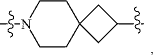

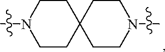

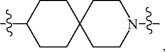

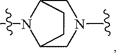

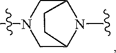

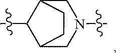

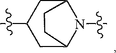

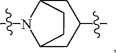

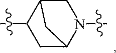

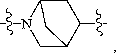

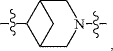

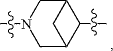

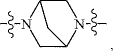

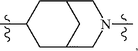

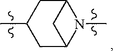

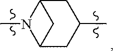

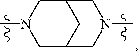

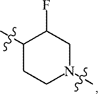

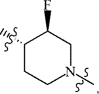

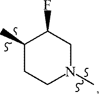

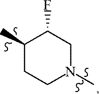

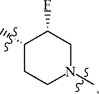

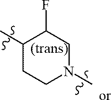

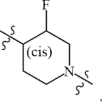

which, when substituted, is optionally further substituted with 0 to 4 substituents selected from H, F, CF₃, methyl, oxo, hydroxymethyl, COOH, CN or NH₂;
in certain embodiments, L is selected from

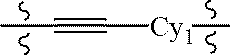

and

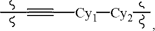

the left side of L is linked to B, and Cy1 and Cy2 are each independently selected from one of the following substituted or unsubstituted groups:

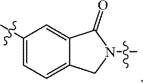

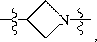

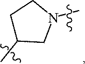

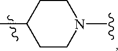

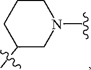

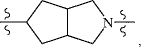

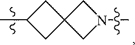

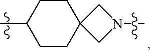

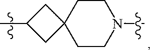

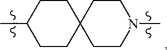

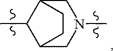

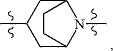

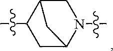

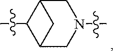

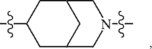

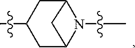

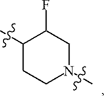

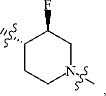

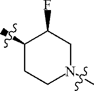

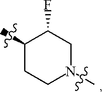

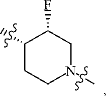

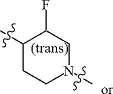

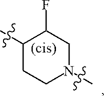

which, when substituted, is optionally further substituted with 0 to 4 substituents selected from H, F, CF₃, methyl, oxo, hydroxymethyl, COOH, CN or NH₂;
in certain embodiments, L is selected from

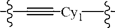

and

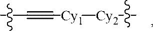

the left side of L is linked to B, and Cy1 and Cy2 are each independently selected from one of the following substituted or unsubstituted groups:

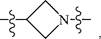

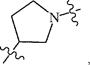

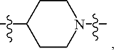

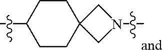

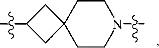

which, when substituted, is optionally further substituted with 0 to 4 substituents selected from H, F, CF₃, methyl, oxo, hydroxymethyl, COOH, CN or NH₂;
in certain embodiments, L is selected from a bond,

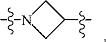

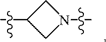

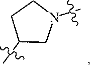

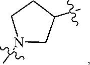

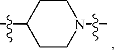

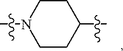

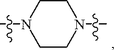

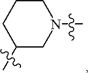

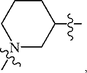

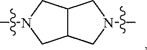

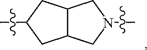

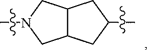

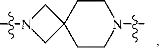

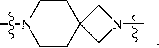

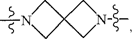

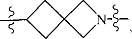

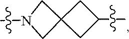

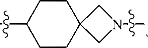

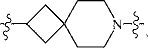

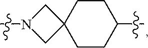

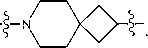

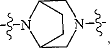

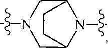

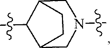

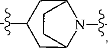

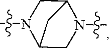

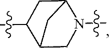

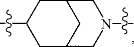

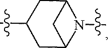

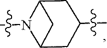

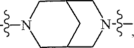

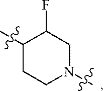

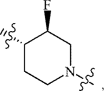

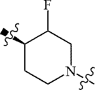

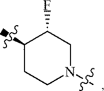

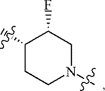

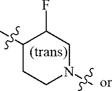

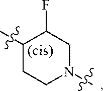

wherein the left side of L is linked to B;
or L is selected from

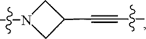

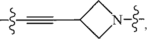

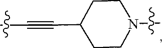

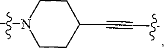

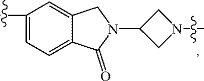

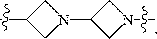

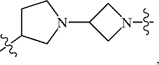

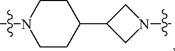

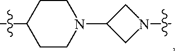

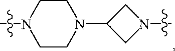

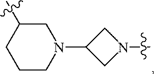

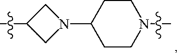

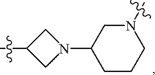

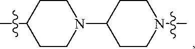

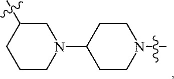

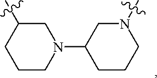

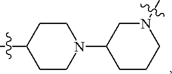

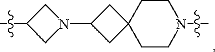

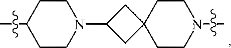

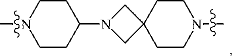

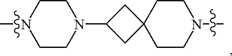

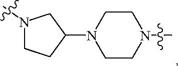

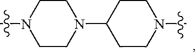

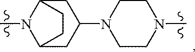

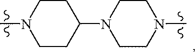

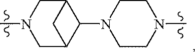

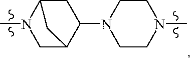

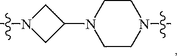

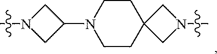

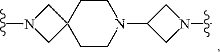

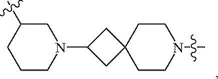

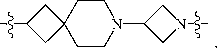

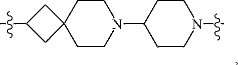

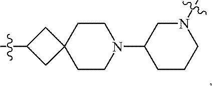

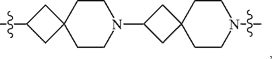

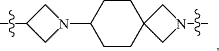

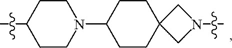

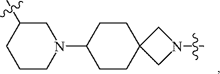

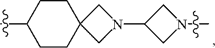

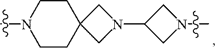

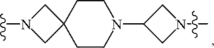

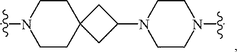

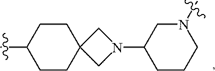

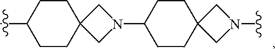

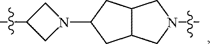

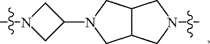

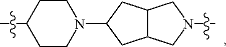

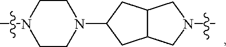

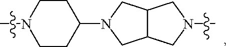

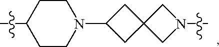

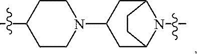

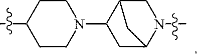

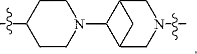

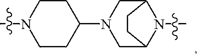

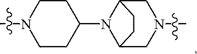

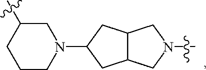

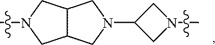

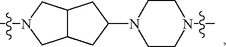

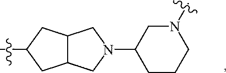

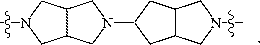

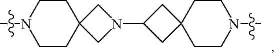

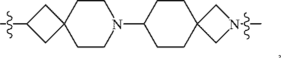

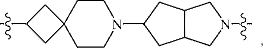

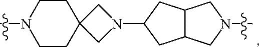

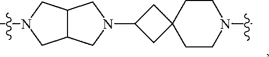

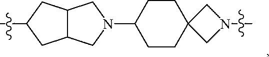

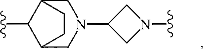

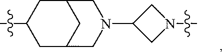

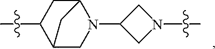

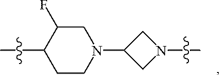

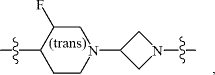

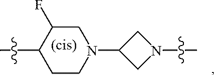

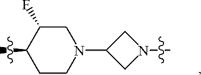

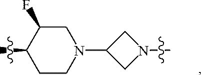

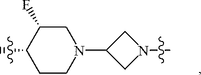

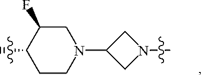

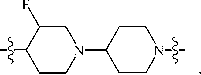

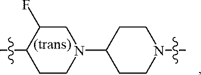

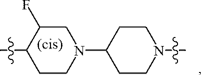

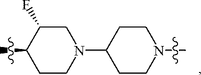

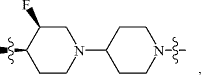

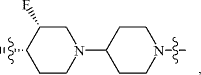

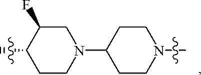

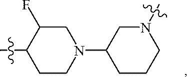

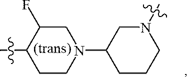

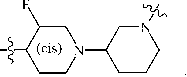

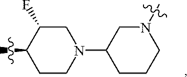

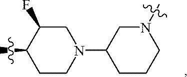

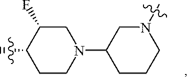

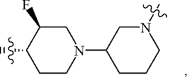

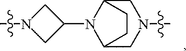

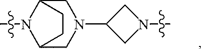

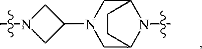

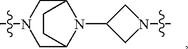

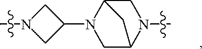

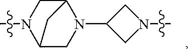

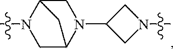

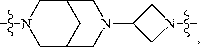

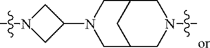

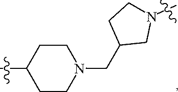

wherein the left side of L is linked to B;
or L is selected from

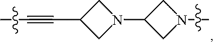

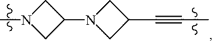

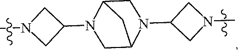

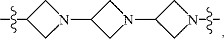

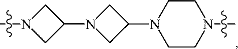

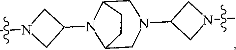

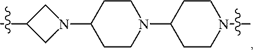

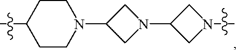

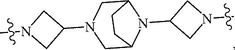

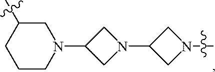

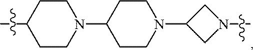

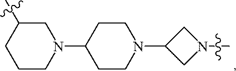

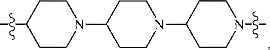

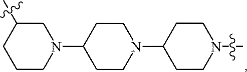

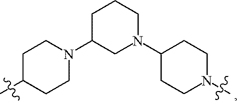

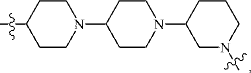

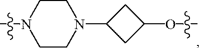

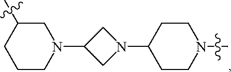

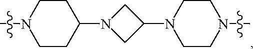

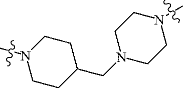

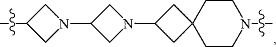

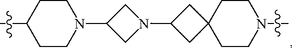

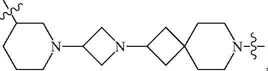

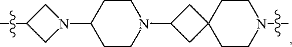

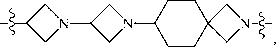

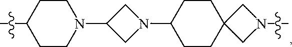

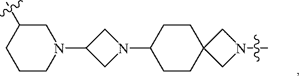

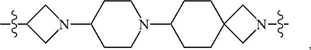

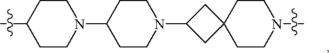

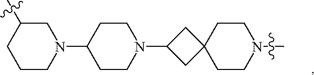

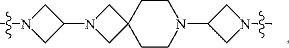

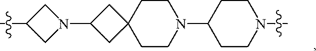

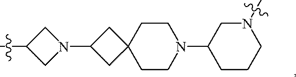

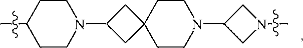

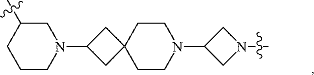

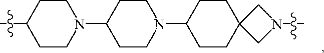

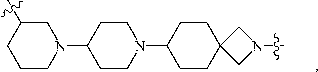

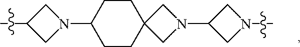

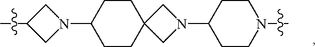

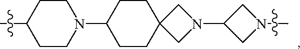

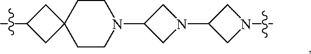

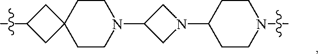

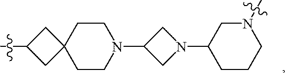

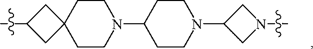

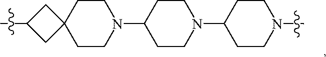

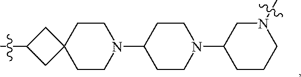

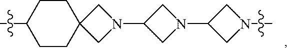

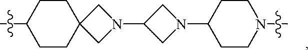

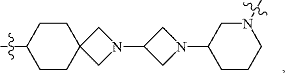

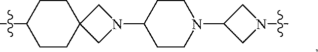

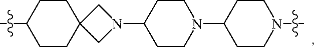

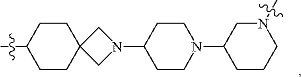

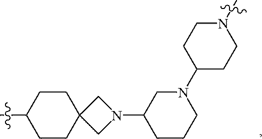

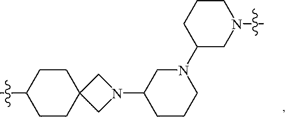

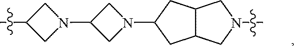

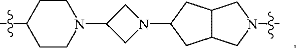

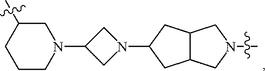

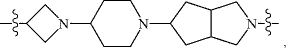

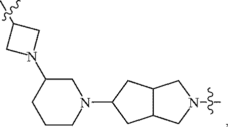

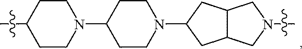

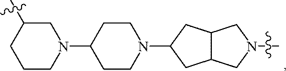

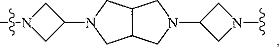

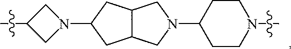

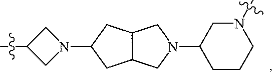

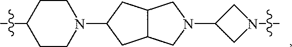

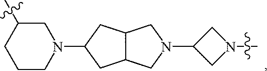

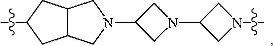

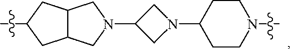

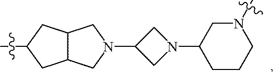

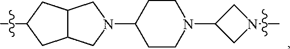

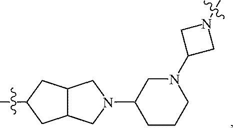

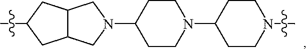

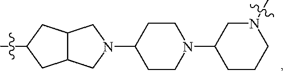

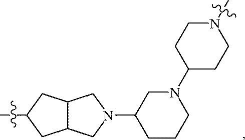

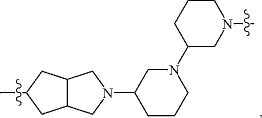

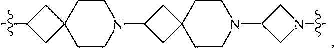

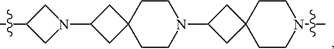

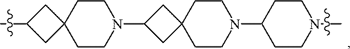

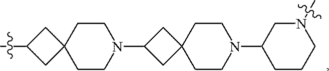

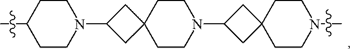

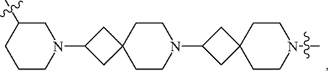

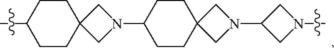

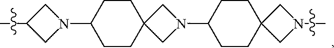

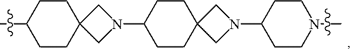

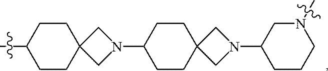

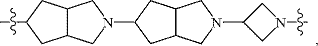

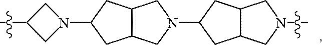

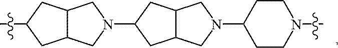

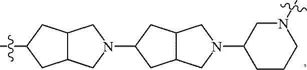

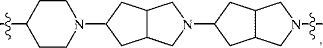

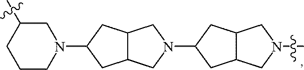

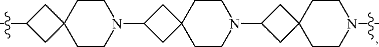

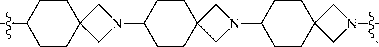

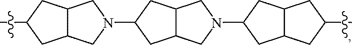

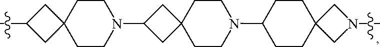

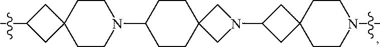

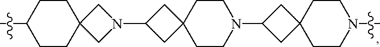

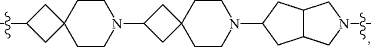

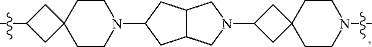

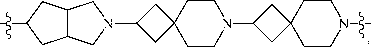

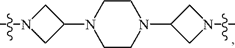

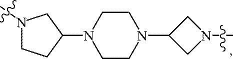

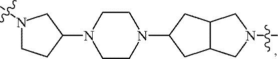

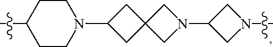

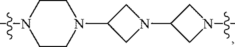

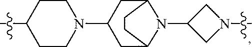

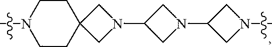

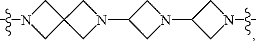

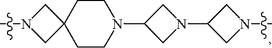

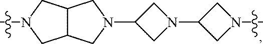

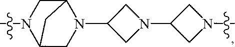

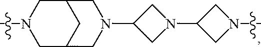

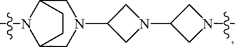

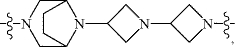

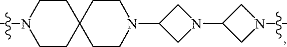

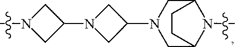

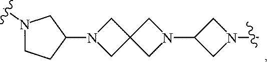

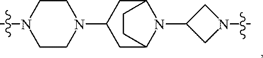

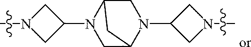

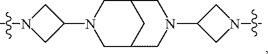

wherein the left side of L is linked to B;
or L is selected from

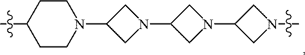

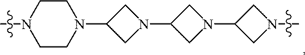

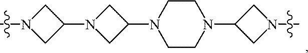

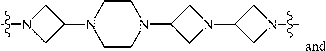

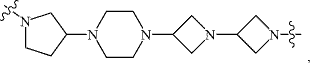

wherein the left side of L is linked to B;
in certain embodiments, K is selected from

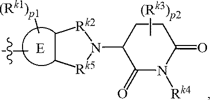

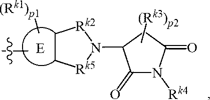

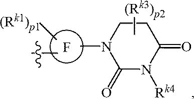

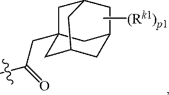

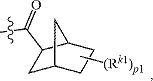

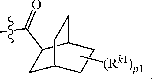

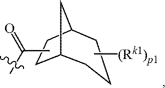

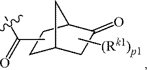

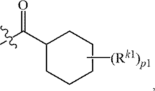

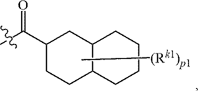

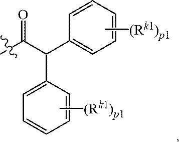

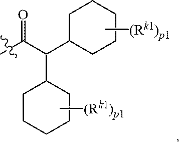

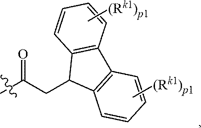

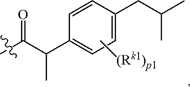

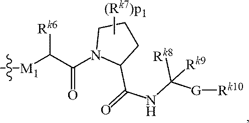

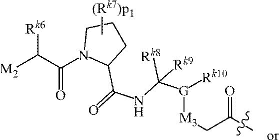

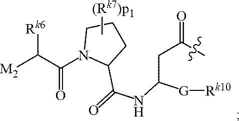

ring E or F is each independently selected from a phenyl ring or a 5- to 6-membered heteroaryl ring, wherein the heteroaryl ring contains 1 to 2 heteroatoms selected from O, S and N;
each R^{k2} is independently selected from CH₂, C═O, S═O and SO₂;
R^{k1}, R^{k3} or R^{k4} is each independently selected from H, F, Cl, Br, I, OH, NH₂, CF₃, CN, COOH, C₁₋₄ alkyl or C₁₋₄ alkoxy;
R^{k5} is selected from C═O or

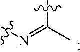

M₁ is selected from a bond, —CH₂—C(═O)NH— or —C(═O)CH₂NH—;
M₂ is selected from -NHC(=O)-C₁₋₆ alkyl or -NHC(=O)-C₃₋₆ cycloalkyl, wherein the alkyl or cycloalkyl is optionally further substituted with 0 to 4 substituents selected from H, F, Cl, Br, I, OH, NH₂, C₁₋₄ alkyl or C₁₋₄ alkoxy;
M₃ is selected from —NH— or —O—;
R^{k6} is selected from C₁₋₆ alkyl, wherein the alkyl is optionally further substituted with 0 to 4 substituents selected from H, F, Cl, Br, I, OH, C₁₋₆ alkyl or C₃₋₆ cycloalkyl;
each R^{k7} is independently selected from H, F, Cl, Br, I, OH, SH, C₁₋₆ alkyl, C₁₋₆ alkoxy, C₁₋₆ alkylthio or C₁₋₆ alkylformyloxy, wherein the alkyl, alkoxy or alkylthio is optionally further substituted with 0 to 4 substituents selected from H, F, Cl, Br, I, OH, C₁₋₄ alkyl or C₁₋₄ alkoxy;
R^{k8} and R^{k9} are each independently selected from H, C₁₋₆ alkyl or C₃₋₆ cycloalkyl, wherein the alkyl or cycloalkyl is optionally further substituted with 0 to 4 substituents selected from H, F, Cl, Br, I, OH, NH₂, C₁₋₄ alkyl or C₁₋₄ alkoxy;
R^{k10} is selected from 5- to 6-membered heteroaryl, wherein the heteroaryl is optionally further substituted with 0 to 4 substituents selected from H, F, Cl, Br, I, OH, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl;
G is selected from 6- to 10-membered aryl or 5- to 10-membered heteroaryl, wherein the aryl or heteroaryl is optionally further substituted with 0 to 4 substituents selected from H, F, Cl, Br, I, OH, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl; and
p1 or p2 is each independently selected from 0, 1, 2, 3 or 4;
in certain embodiments, K is selected from

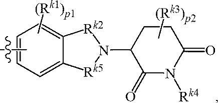

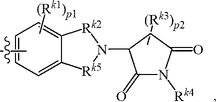

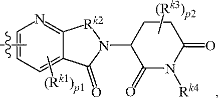

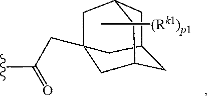

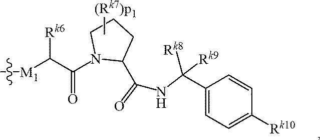

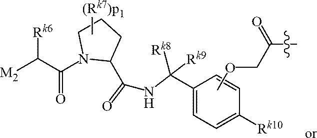

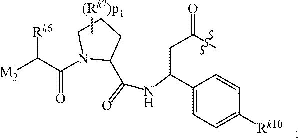

in certain embodiments, K is selected from

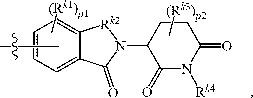

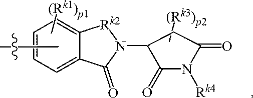

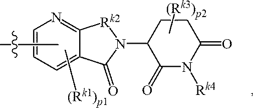

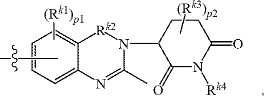

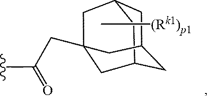

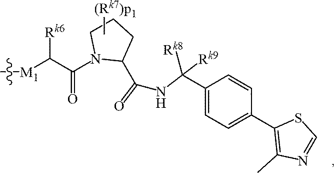

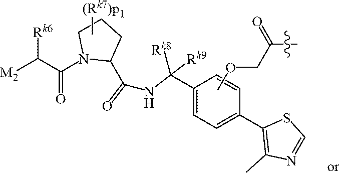

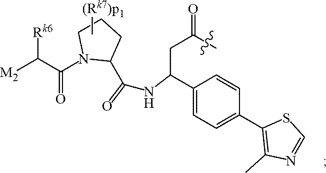

each R^{k2} is independently selected from CH₂ or C═O;
R^{k1}, R^{k3} or R^{k4} is each independently selected from H, CH₃, F, Cl, Br, I, OH or NH₂;
M₁ is selected from a bond, —CH₂—C(═O)NH— or —C(═O)CH₂NH—;
M₂ is selected from -NHC(=O)-methyl, -NHC(=O)-ethyl, -NHC(=O)-cyclopropyl, -NHC(=O)-cyclobutyl, -NHC(=O)-cyclopentyl or -NHC(=O)-cyclohexyl, wherein the methyl, ethyl, cyclopropyl, cyclobutyl, cyclopentyl or cyclohexyl is optionally further substituted with 0 to 4 substituents selected from H, F, Cl, Br, I, OH, NH₂, C₁₋₄ alkyl or C₁₋₄ alkoxy;
R^{k6} is selected from methyl, ethyl, propyl, isopropyl, tert-butyl, isobutyl or sec-butyl;
each R^{k7} is independently selected from H, F, OH, SH, methyl, methoxy or —SCH₃;
R^{k8} and R^{k9} are each independently selected from H, methyl, ethyl, cyclopropyl or cyclobutyl; and
p1 or p2 is each independently selected from 0, 1 or 2;
in certain embodiments, K is selected from

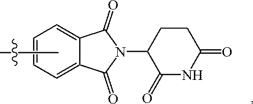

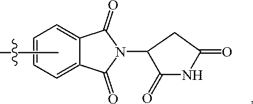

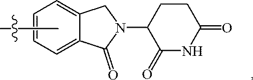

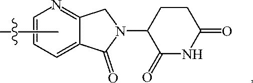

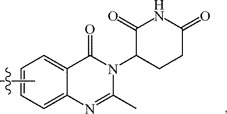

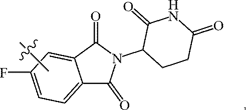

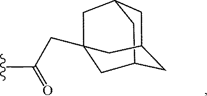

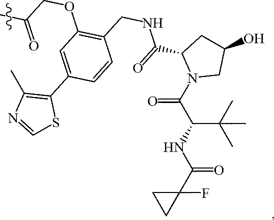

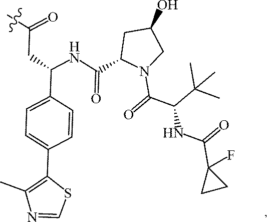

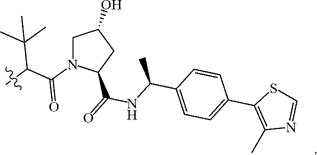

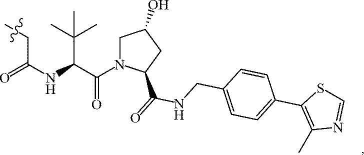

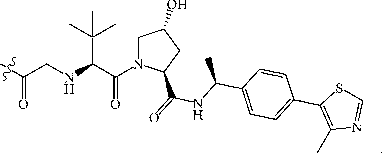

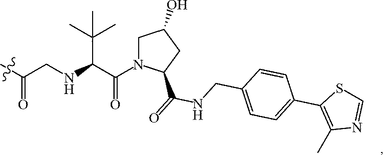

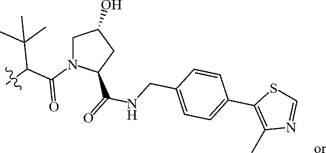

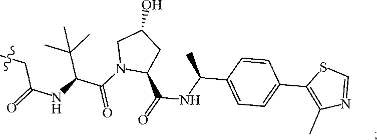

in certain embodiments, K is selected from

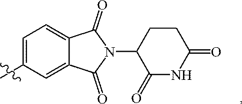

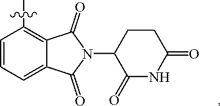

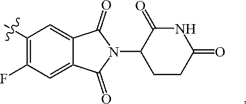

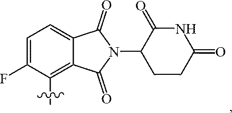

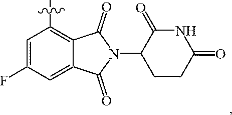

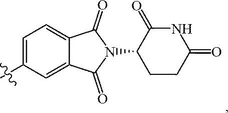

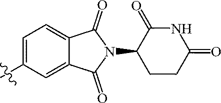

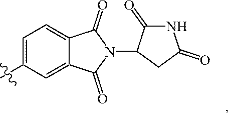

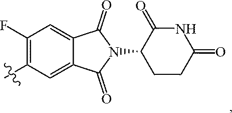

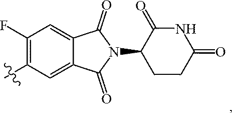

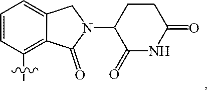

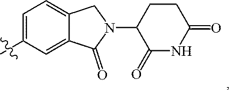

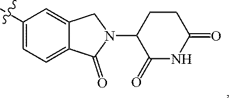

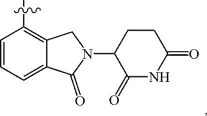

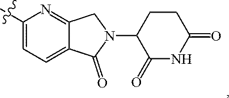

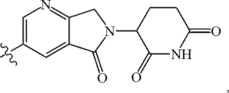

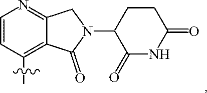

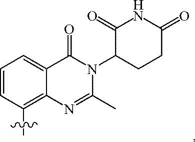

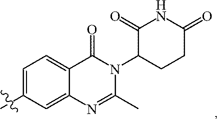

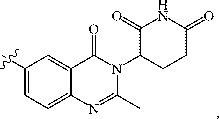

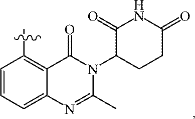

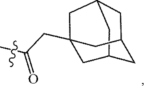

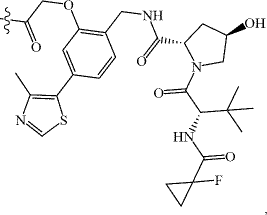

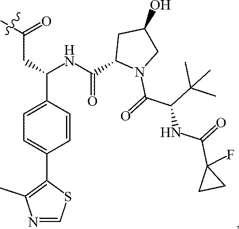

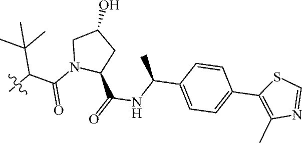

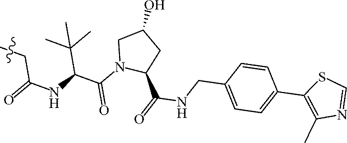

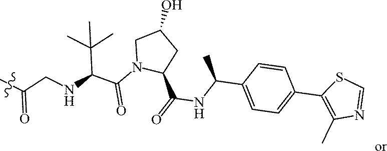

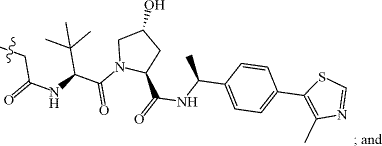

in certain embodiments, K is selected from

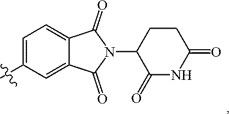

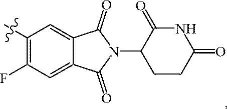

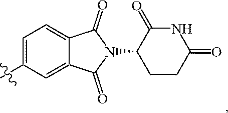

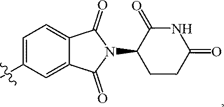

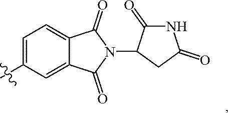

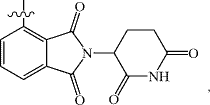

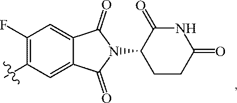

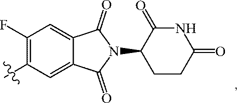

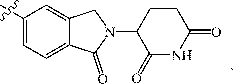

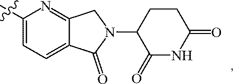

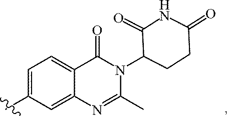

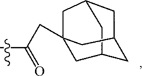

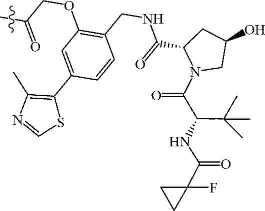

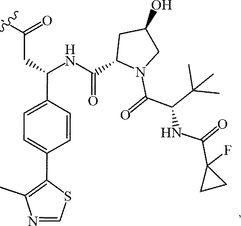

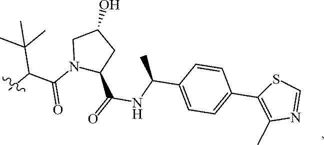

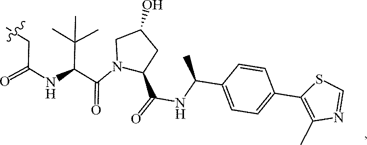

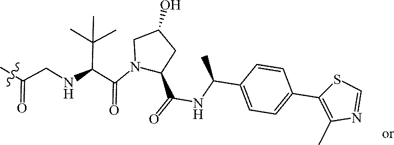

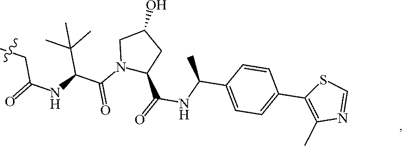

As a first embodiment of the present disclosure, provided is a compound as shown in general formula (I) or a stereoisomer, a deuterated compound, a solvate, a prodrug, a metabolite, a pharmaceutically acceptable salt or a co-crystal thereof, wherein

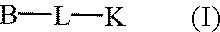

L is selected from -Ak1-Cy1-Ak2-Cy2-Ak3-Cy3-Ak4-Cy4-Ak5-;
Ak1, Ak2, Ak3, Ak4 and Ak5 are each independently selected from CH₂, O, C≡C or a bond;
Cy1, Cy2, Cy3 and Cy4 are each independently selected from a bond, a 4- to 7-membered mono-heterocyclic ring, a 5- to 10-membered fused heterocyclic ring, a 6- to 12-membered spiro-heterocyclic ring, a 7- to 10-membered bridged-heterocyclic ring, 4- to 7-membered monocycloalkyl, 5- to 10-membered fused cycloalkyl, 6- to 12-membered spiro cycloalkyl, 7- to 10-membered bridged cycloalkyl, 5- to 10-membered heteroaryl or 6- to 10-membered aryl, wherein the aryl, heteroaryl, cycloalkyl, mono-heterocyclic ring, fused heterocyclic ring, spiro-heterocyclic ring or bridged-heterocyclic ring is optionally further substituted with 0 to 4 substituents selected from H, F, Cl, Br, I, OH, COOH, CN, NH₂, oxo, C₁₋₄ alkyl, halogen-substituted C₁₋₄ alkyl, hydroxyl-substituted C₁₋₄ alkyl or C₁₋₄ alkoxy, and the heteroaryl, mono-heterocyclic ring, fused heterocyclic ring, spiro-heterocyclic ring or bridged-heterocyclic ring contains 1 to 4 heteroatoms selected from O, S and N;
B is selected from

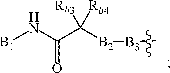

B₁ is selected from one of the following substituted or unsubstituted groups: 6-membered aryl or 6-membered heteroaryl, which, when substituted, is optionally further substituted with 0 to 4 R^{b1}, wherein the heteroaryl contains 1 to 4 heteroatoms selected from O, S and N;
B₂ is selected from one of the following substituted or unsubstituted groups: a 5-to 10-membered heterocyclic group or —NHC(═O)—, which, when substituted, is optionally further substituted with 0 to 4 R^{b2}, wherein the heterocyclic group contains 1 to 4 heteroatoms selected from O, S and N;
B₃ is selected from substituted or unsubstituted 5- to 6-membered aryl or a bond, wherein the 5- to 6-membered aryl, when substituted, is optionally further substituted with 0 to 4 R^{b2};
R^{b1} and R^{b2} are each independently selected from H, F, Cl, Br, I, OH, NH₂, CN, CF₃, —C(═O)NH₂, —C(═O)NH—C₁₋₄ alkyl, —C(═O)N(C₁₋₄ alkyl)₂, C₁₋₄ alkyl or C₁₋₄ alkoxy, wherein the alkyl or alkoxy is optionally further substituted with 0 to 4 substituents selected from H, F, Cl, Br, I or OH;
R^{b3} and R^{b4} are each independently selected from H or C₁₋₆ alkyl, wherein the alkyl is optionally further substituted with 0 to 4 substituents selected from H, F, Cl, Br, I, OH, NH₂, C₁₋₄ alkyl or C₁₋₄ alkoxy;
or R^{b3} and R^{b4} together with the carbon atoms to which they are attached form C₃₋₆ cycloalkyl or a C₃₋₆ mono-heterocyclic ring, wherein the cycloalkyl or mono-heterocyclic ring is optionally further substituted with 0 to 4 substituents selected from H, F, Cl, Br, I, OH, NH₂, C₁₋₄ alkyl or C₁₋₄ alkoxy, and the mono-heterocyclic ring contains 1 to 4 heteroatoms selected from O, S and N;
K is selected from

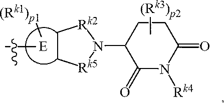

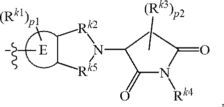

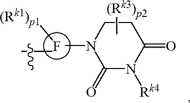

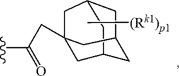

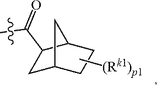

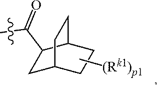

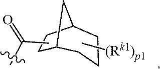

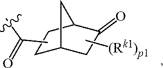

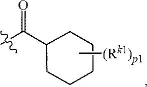

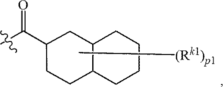

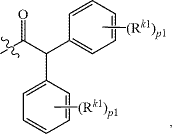

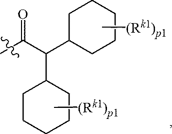

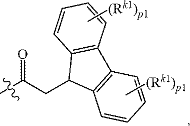

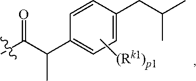

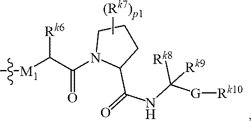

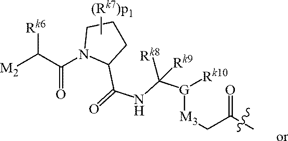

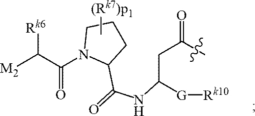

ring E or F is each independently selected from a phenyl ring or a 5- to 6-membered heteroaryl ring, wherein the heteroaryl ring contains 1 to 2 heteroatoms selected from O, S and N;
each R^{k2} is independently selected from CH₂, C═O, S═O and SO₂;
R^{k1}, R^{k3} or R^{k4} is each independently selected from H, F, Cl, Br, I, OH, NH₂, CF₃, CN, COOH, C₁₋₄ alkyl or C₁₋₄ alkoxy;
R^{k5} is selected from C═O or

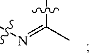

M₁ is selected from a bond, —CH₂—C(═O)NH— or —C(═O)CH₂NH—;
M₂ is selected from -NHC(=O)-C₁₋₆ alkyl or -NHC(=O)-C₃₋₆ cycloalkyl, wherein the alkyl or cycloalkyl is optionally further substituted with 0 to 4 substituents selected from H, F, Cl, Br, I, OH, NH₂, C₁₋₄ alkyl or C₁₋₄ alkoxy;
M₃ is selected from —NH— or —O—;
R^{k6} is selected from C₁₋₆ alkyl, wherein the alkyl is optionally further substituted with 0 to 4 substituents selected from H, F, Cl, Br, I, OH, C₁₋₆ alkyl or C₃₋₆ cycloalkyl;
each R^{k7} is independently selected from H, F, Cl, Br, I, OH, SH, C₁₋₆ alkyl, C₁₋₆ alkoxy, C₁₋₆ alkylthio or C₁₋₆ alkylformyloxy, wherein the alkyl, alkoxy or alkylthio is optionally further substituted with 0 to 4 substituents selected from H, F, Cl, Br, I, OH, C₁₋₄ alkyl or C₁₋₄ alkoxy;
R^{k8} and R^{k9} are each independently selected from H, C₁₋₆ alkyl or C₃₋₆ cycloalkyl, wherein the alkyl or cycloalkyl is optionally further substituted with 0 to 4 substituents selected from H, F, Cl, Br, I, OH, NH₂, C₁₋₄ alkyl or C₁₋₄ alkoxy;
R^{k10} is selected from 5- to 6-membered heteroaryl, wherein the heteroaryl is optionally further substituted with 0 to 4 substituents selected from H, F, Cl, Br, I, OH, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl;
G is selected from 6- to 10-membered aryl or 5- to 10-membered heteroaryl, wherein the aryl or heteroaryl is optionally further substituted with 0 to 4 substituents selected from H, F, Cl, Br, I, OH, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl; and
p1 or p2 is each independently selected from 0, 1, 2, 3 or 4.

As a second embodiment of the present disclosure, provided is a compound as shown in general formula (I) or a stereoisomer, a deuterated compound, a solvate, a prodrug, a metabolite, a pharmaceutically acceptable salt or a co-crystal thereof, wherein
Cy1, Cy2, Cy3 and Cy4 are each independently selected from a bond, a 4- to 7-membered nitrogen-containing mono-heterocyclic ring, a 5- to 10-membered nitrogen-containing fused heterocyclic ring, a 7- to 10-membered bridged-heterocyclic ring, or a 6- to 12-membered nitrogen-containing spiro-heterocyclic ring, wherein the mono-heterocyclic ring, fused heterocyclic ring, bridged-heterocyclic ring or spiro-heterocyclic ring is optionally further substituted with 0 to 4 substituents selected from H, F, Cl, Br, I, OH, COOH, CN, NH₂, oxo, C₁₋₄ alkyl, halogen-substituted C₁₋₄ alkyl, hydroxyl-substituted C₁₋₄ alkyl or C₁₋₄ alkoxy, and the mono-heterocyclic ring, fused heterocyclic ring, bridged-heterocyclic ring or spiro-heterocyclic ring contains 1 to 4 heteroatoms selected from O, S and N; and
K is selected from

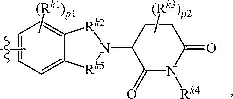

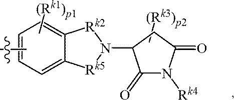

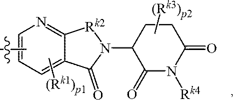

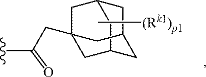

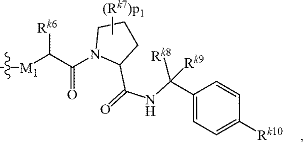

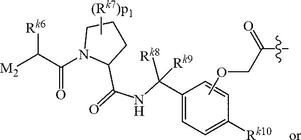

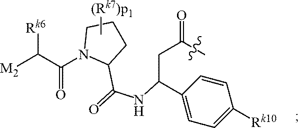

other groups are as defined in the first embodiment.

As a third embodiment of the present disclosure, provided is a compound as shown in general formula (I) or a stereoisomer, a deuterated compound, a solvate, a prodrug, a metabolite, a pharmaceutically acceptable salt or a co-crystal thereof, wherein
Cy1, Cy2, Cy3 and Cy4 are each independently selected from a bond or one of the following substituted or unsubstituted groups: azetidinyl, azacyclopentyl, azacyclohexyl, piperidine, morpholine, piperazine, cyclopropyl-fused-azetidinyl, cyclopropyl-fused-azacyclopentyl, cyclopropyl-fused-azacyclohexyl, cyclopropyl-fused-piperidine, cyclobutyl-fused-azetidinyl, cyclobutyl-fused-azacyclopentyl, cyclobutyl-fused-azacyclohexyl, cyclobutyl-fused-piperidine, cyclopentyl-fused-azetidinyl, cyclopentyl-fused-azacyclopentyl, cyclopentyl-fused-azacyclohexyl, cyclopentyl-fused-piperidine, cyclohexyl-fused-azetidinyl, cyclohexyl-fused-azacyclopentyl, cyclohexyl-fused-azacyclohexyl, cyclohexyl-fused-piperidine, azetidinyl-fused-azetidinyl, azetidinyl-fused-azacyclopentyl, azetidinyl-fused-azacyclohexyl, azetidinyl-fused-piperidine, azacyclopentyl-fused-azetidinyl, azacyclopentyl-fused-azacyclopentyl, azacyclopentyl-fused-azacyclohexyl, azacyclopentyl-fused-piperidine, azacyclohexyl-fused-azetidinyl, azacyclohexyl-fused-azacyclopentyl, azacyclohexyl-fused-azacyclohexyl, azacyclohexyl-fused-piperidine, cyclobutyl-spiro-azetidinyl, cyclobutyl-spiro-azacyclopentyl, cyclobutyl-spiro-azacyclohexyl, cyclopentyl-spiro-azetidinyl, cyclopentyl-spiro-azacyclopentyl, cyclopentyl-spiro-azacyclohexyl, cyclohexyl-spiro-azetidinyl, cyclohexyl-spiro-azacyclopentyl, cyclohexyl-spiro-azacyclohexyl, azetidinyl-spiro-azetidinyl, azetidinyl-spiro-azacyclopentyl, azetidinyl-spiro-azacyclohexyl, azacyclopentyl-spiro-azetidinyl, azacyclopentyl-spiro-azacyclopentyl, azacyclopentyl-spiro-azacyclohexyl, azacyclohexyl-spiro-azetidinyl, azacyclohexyl-spiro-azacyclopentyl, azacyclohexyl-spiro-azacyclohexyl, cyclobutyl-spiro-piperidine, cyclopentyl-spiro-piperidine, cyclohexyl- spiro-piperidine, azetidinyl- spiro-piperidine, azacyclopentyl- spiro-piperidine, azacyclohexyl-spiro-piperidine,

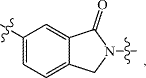

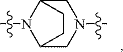

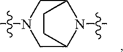

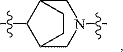

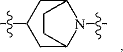

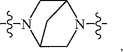

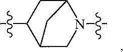

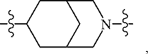

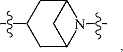

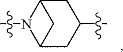

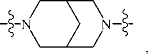

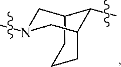

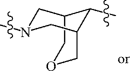

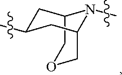

which, when substituted, is optionally further substituted with 0 to 4 substituents selected from H, F, Cl, Br, I, OH, NH₂, COOH, CN, oxo, C₁₋₄ alkyl, halogen-substituted C₁₋₄ alkyl, hydroxyl-substituted C₁₋₄ alkyl or C₁₋₄ alkoxy;
R^{b1} and R^{b2} are each independently selected from H, F, Cl, Br, I, OH, NH₂, CN, CF₃, —C(═O)NH₂, —C(═O)NH— CH₃, —C(═O)N(CH₃)₂, methyl, ethyl, propyl, isopropyl, methoxy or ethoxy, wherein the methyl, ethyl, propyl, isopropyl, methoxy or ethoxy is optionally further substituted with 0 to 4 substituents selected from H, F, Cl, Br, I or OH;
K is selected from

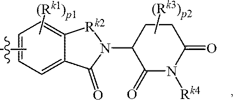

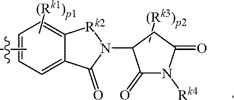

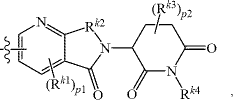

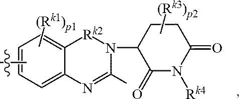

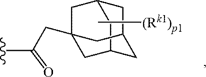

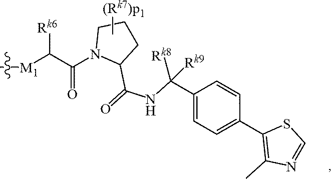

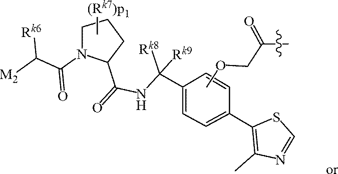

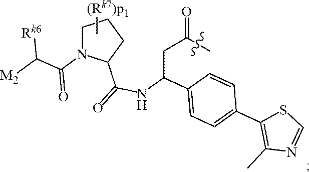

each R^{k2} is independently selected from CH₂ or C═O;
R^{k1}, R^{k3} or R^{k4} is each independently selected from H, CH₃, F, Cl, Br, I, OH or NH₂;
M₁ is selected from a bond, —CH₂—C(═O)NH— or —C(═O)CH₂NH—;
M₂ is selected from -NHC(=O)-methyl, -NHC(=O)-ethyl, -NHC(=O)-cyclopropyl, -NHC(=O)-cyclobutyl, -NHC(=O)-cyclopentyl or -NHC(=O)-cyclohexyl, wherein the methyl, ethyl, cyclopropyl, cyclobutyl, cyclopentyl or cyclohexyl is optionally further substituted with 0 to 4 substituents selected from H, F, Cl, Br, I, OH, NH₂, C₁₋₄ alkyl or C₁₋₄ alkoxy;
R^{k6} is selected from methyl, ethyl, propyl, isopropyl, tert-butyl, isobutyl or sec-butyl;
each R^{k7} is independently selected from H, F, OH, SH, methyl, methoxy or -SCH₃;
R^{k8} and R^{k9} are each independently selected from H, methyl, ethyl, cyclopropyl or cyclobutyl; and
p1 or p2 is each independently selected from 0, 1 or 2;
other groups are as defined in the second embodiment.

As a fourth embodiment of the present disclosure, provided is a compound as shown in general formula (I) or a stereoisomer, a deuterated compound, a solvate, a prodrug, a metabolite, a pharmaceutically acceptable salt or a co-crystal thereof, wherein
Cy1, Cy2, Cy3 and Cy4 are each independently selected from one of the following substituted or unsubstituted groups: a bond,

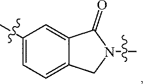

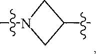

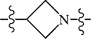

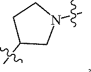

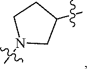

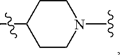

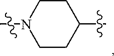

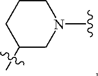

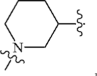

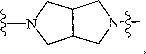

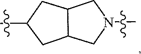

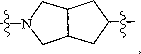

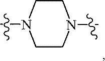

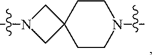

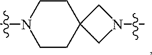

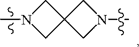

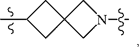

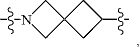

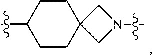

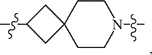

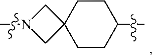

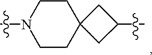

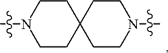

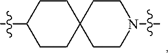

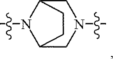

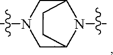

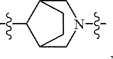

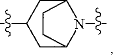

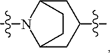

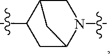

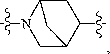

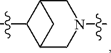

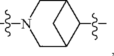

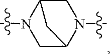

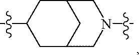

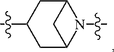

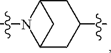

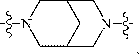

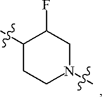

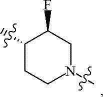

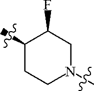

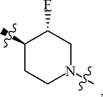

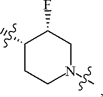

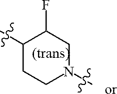

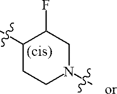

which, when substituted, is optionally further substituted with 0 to 4 substituents selected from H, F, CF₃, methyl, oxo, hydroxymethyl, COOH, CN or NH₂;
B is selected from

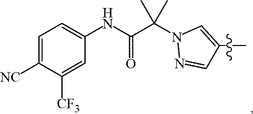

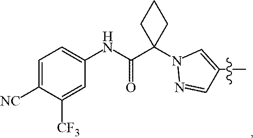

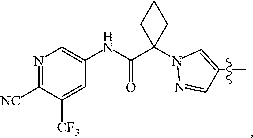

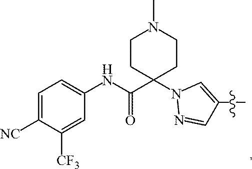

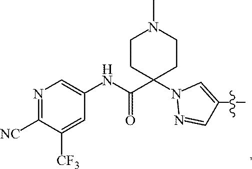

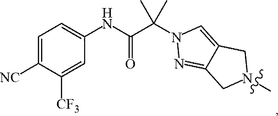

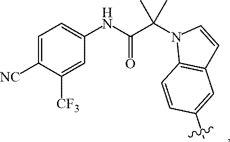

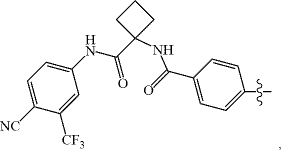

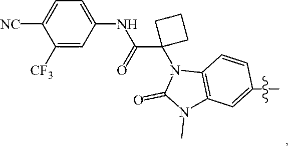

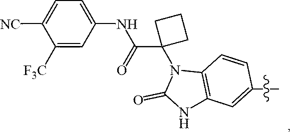

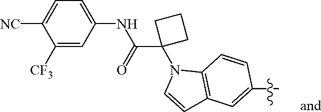

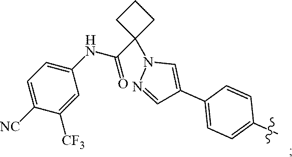

K is selected from

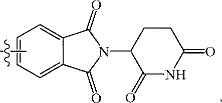

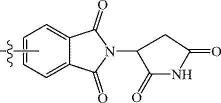

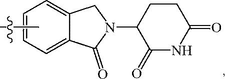

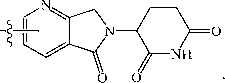

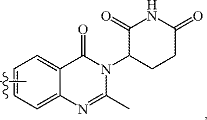

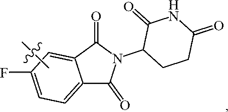

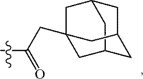

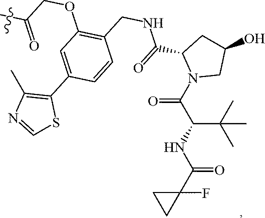

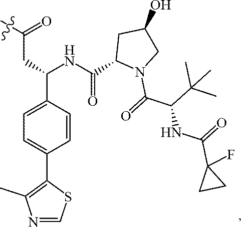

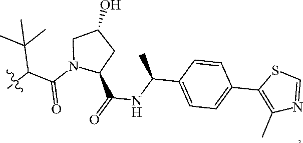

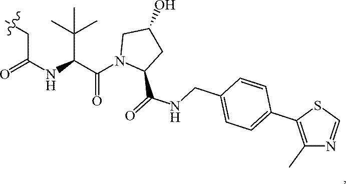

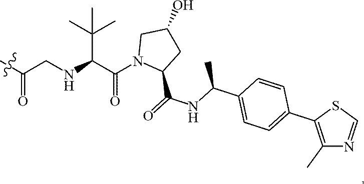

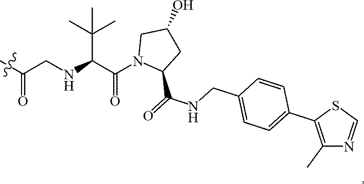

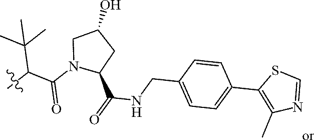

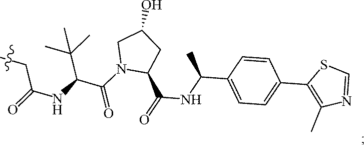

other groups are as defined in the third embodiment.

As a fifth embodiment of the present disclosure, provided is a compound as shown in general formula (I) or a stereoisomer, a deuterated compound, a solvate, a prodrug, a metabolite, a pharmaceutically acceptable salt or a co-crystal thereof, wherein
L is selected from a bond,

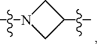

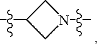

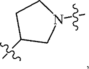

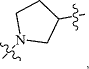

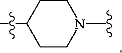

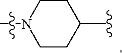

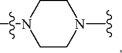

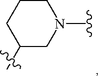

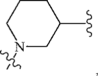

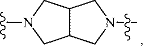

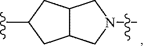

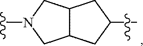

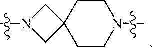

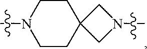

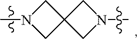

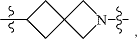

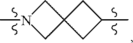

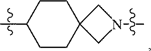

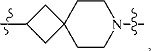

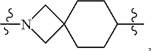

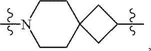

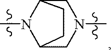

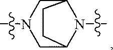

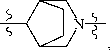

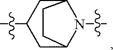

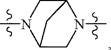

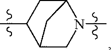

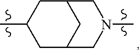

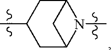

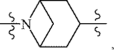

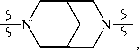

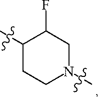

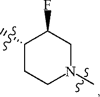

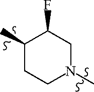

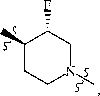

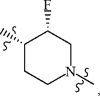

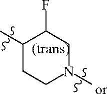

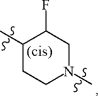

wherein the left side of L is linked to B;
or L is selected from

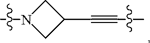

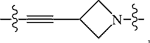

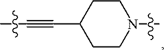

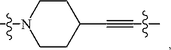

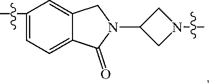

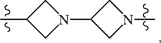

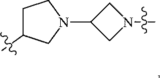

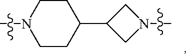

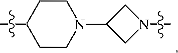

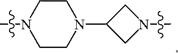

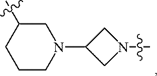

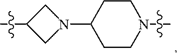

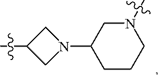

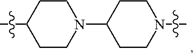

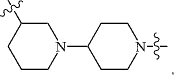

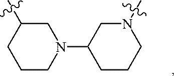

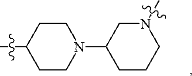

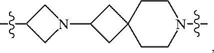

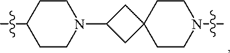

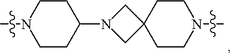

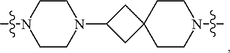

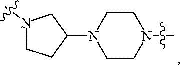

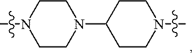

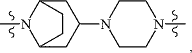

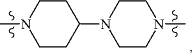

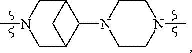

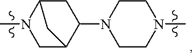

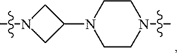

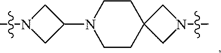

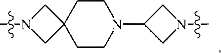

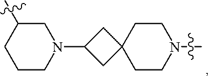

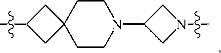

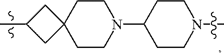

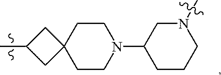

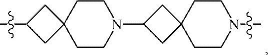

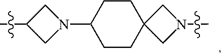

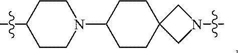

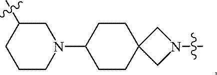

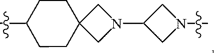

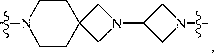

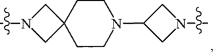

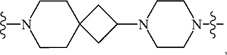

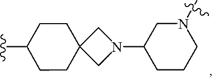

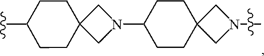

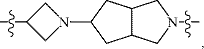

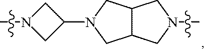

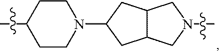

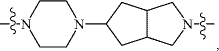

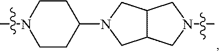

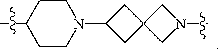

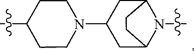

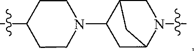

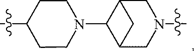

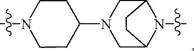

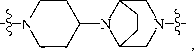

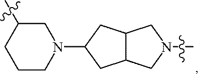

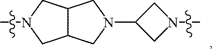

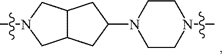

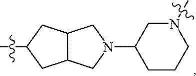

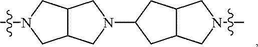

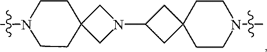

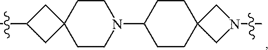

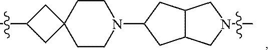

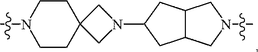

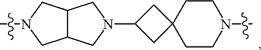

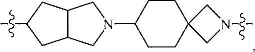

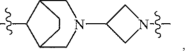

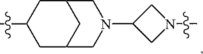

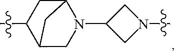

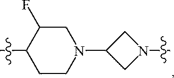

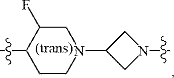

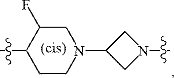

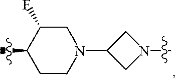

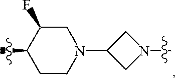

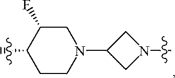

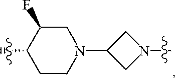

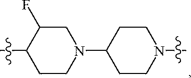

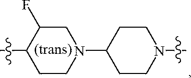

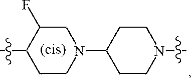

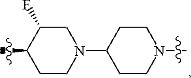

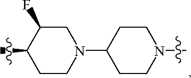

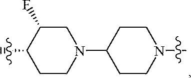

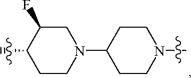

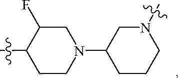

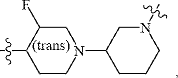

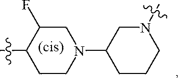

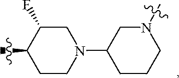

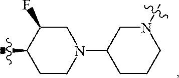

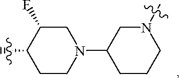

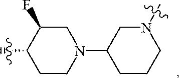

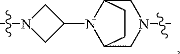

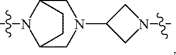

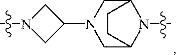

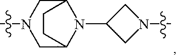

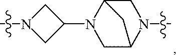

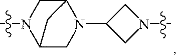

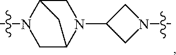

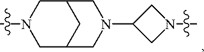

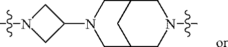

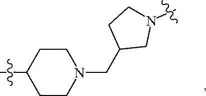

wherein the left side of L is linked to B;
or L is selected from

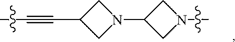

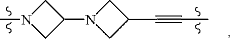

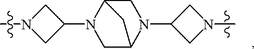

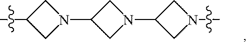

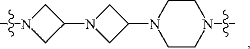

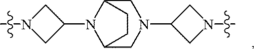

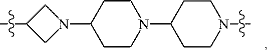

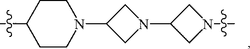

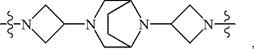

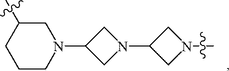

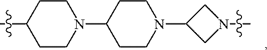

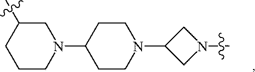

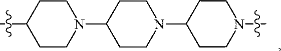

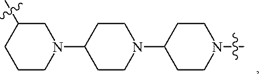

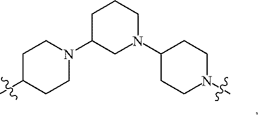

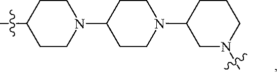

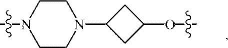

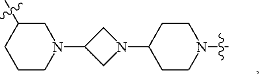

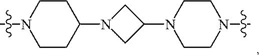

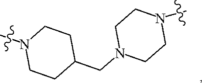

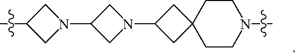

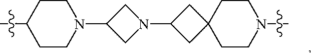

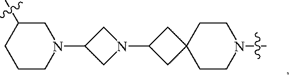

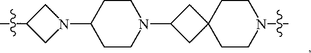

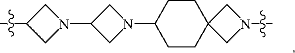

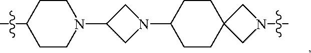

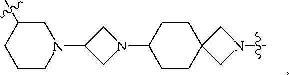

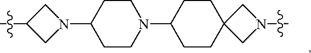

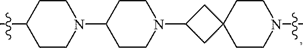

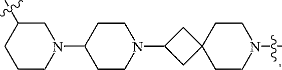

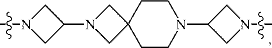

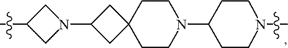

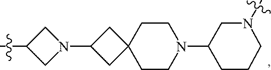

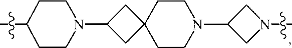

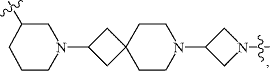

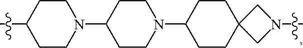

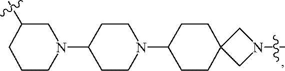

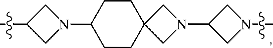

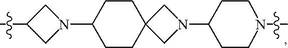

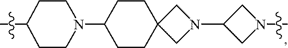

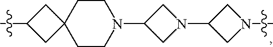

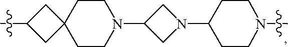

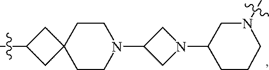

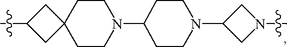

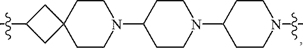

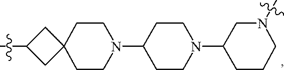

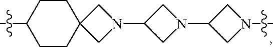

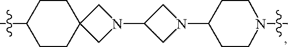

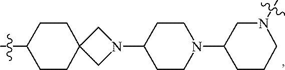

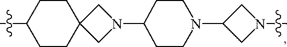

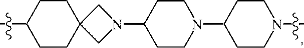

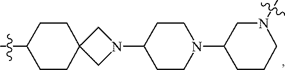

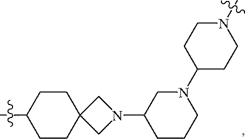

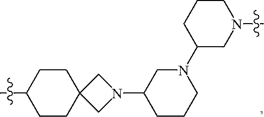

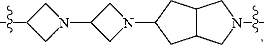

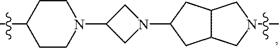

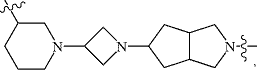

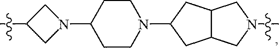

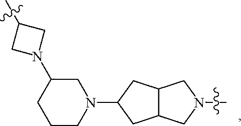

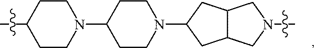

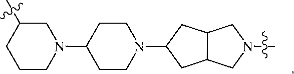

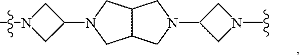

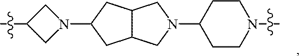

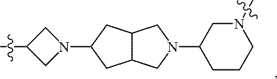

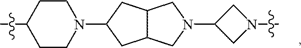

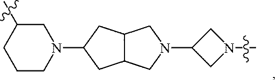

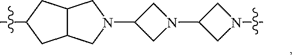

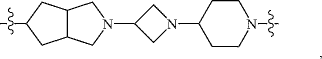

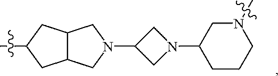

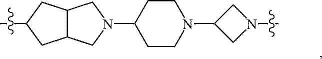

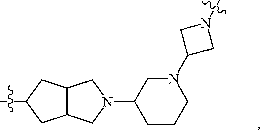

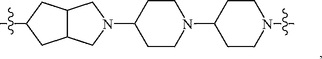

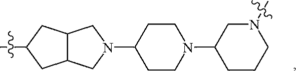

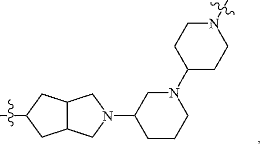

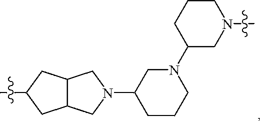

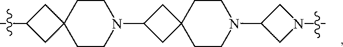

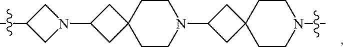

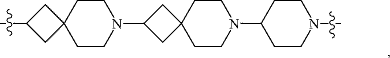

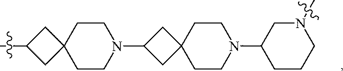

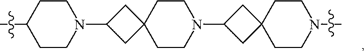

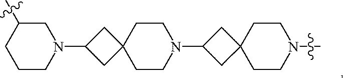

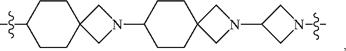

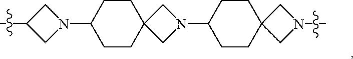

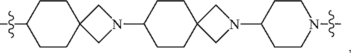

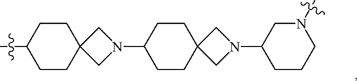

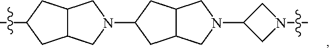

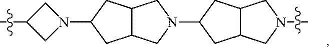

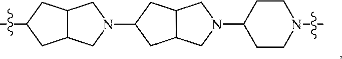

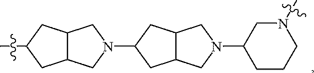

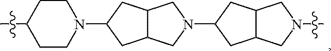

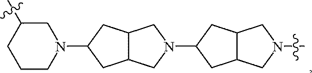

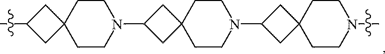

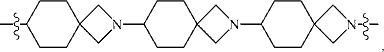

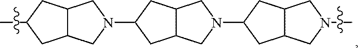

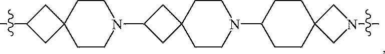

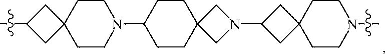

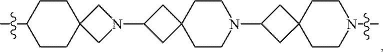

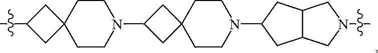

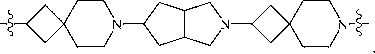

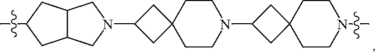

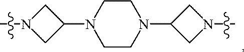

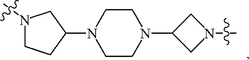

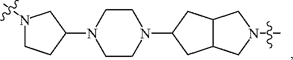

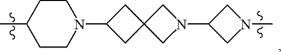

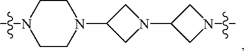

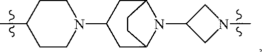

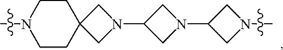

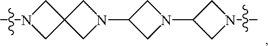

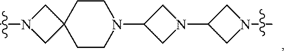

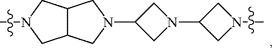

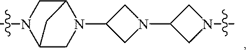

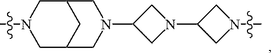

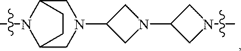

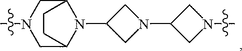

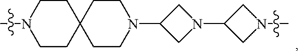

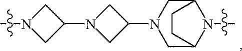

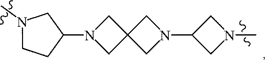

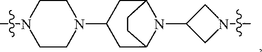

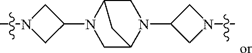

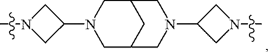

wherein the left side of L is linked to B;
or L is selected from

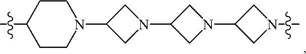

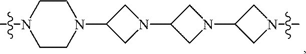

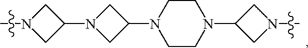

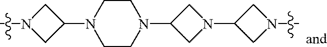

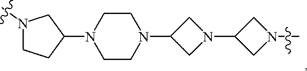

wherein the left side of L is linked to B;
other groups are as defined in the fourth embodiment.

As a sixth embodiment of the present disclosure, provided is a compound as shown in general formula (I) or a stereoisomer, a deuterated compound, a solvate, a prodrug, a metabolite, a pharmaceutically acceptable salt or a co-crystal thereof, wherein
K is selected from

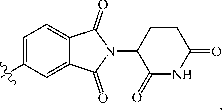

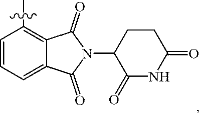

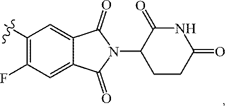

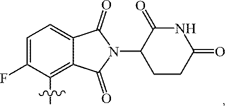

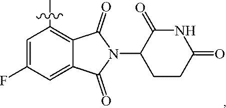

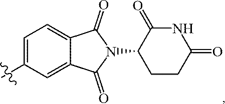

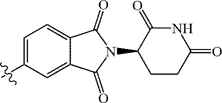

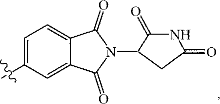

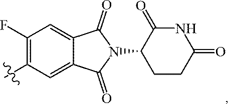

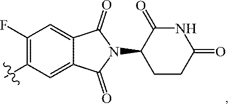

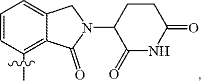

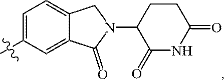

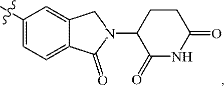

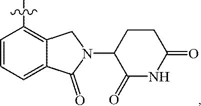

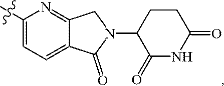

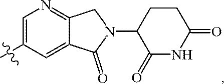

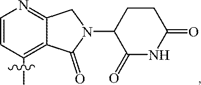

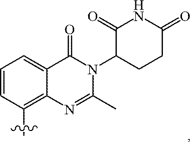

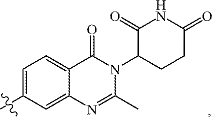

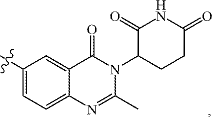

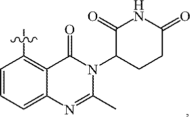

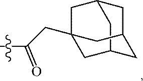

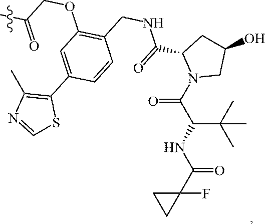

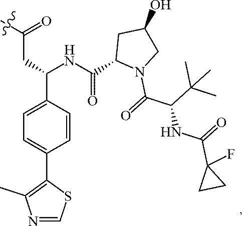

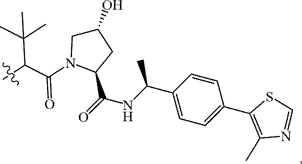

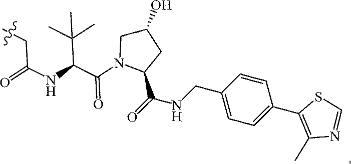

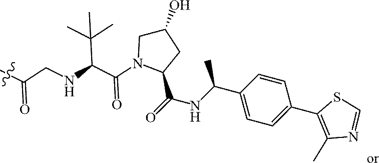

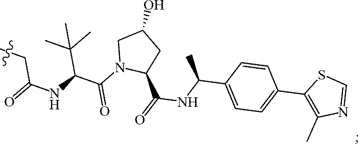

other groups are as defined in the fifth embodiment.

As a seventh embodiment of the present disclosure, provided is a compound as shown in general formula (I) or a stereoisomer, a deuterated compound, a solvate, a prodrug, a metabolite, a pharmaceutically acceptable salt or a co-crystal thereof, wherein
K is selected from

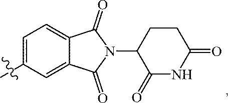

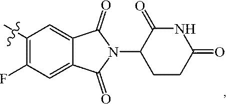

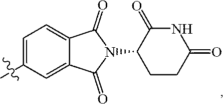

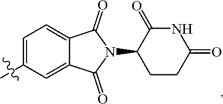

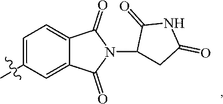

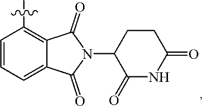

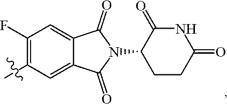

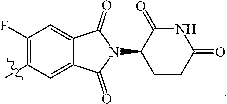

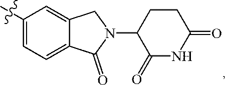

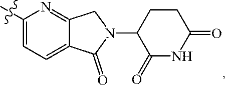

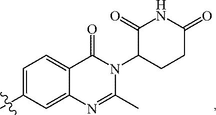

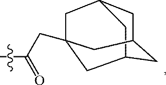

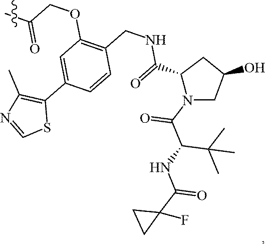

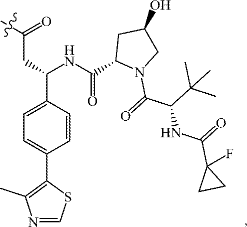

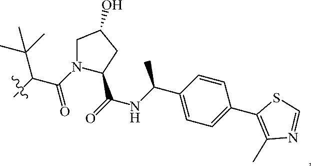

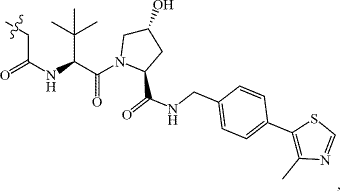

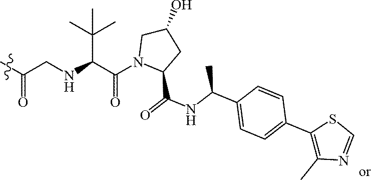

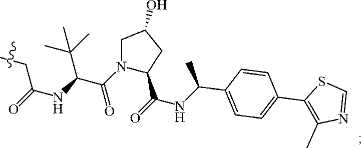

other groups are as defined in the sixth embodiment.

As an embodiment of the present disclosure, provided is a compound as shown in general formula (I) or a stereoisomer, a deuterated compound, a solvate, a prodrug, a metabolite, a pharmaceutically acceptable salt or a co-crystal thereof, wherein the compound has a structure selected from one of the following structures:

| | |
|---|---|
| 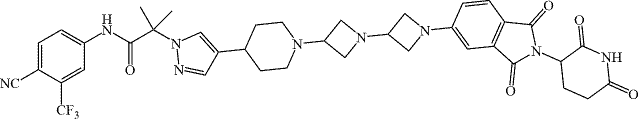 [SMILES: CC(C)(C(=O)Nc1ccc(C#N)c(C(F)(F)F)c1)n1cc(C2CCN(C3CN(C4CN(c5ccc6c(c5)C(=O)N(C5CCC(=O)NC5=O)C6=O)C4)C3)CC2)cn1] | 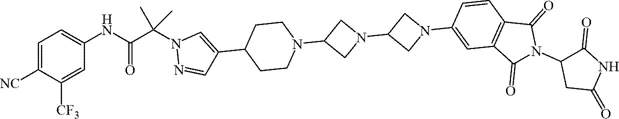 [SMILES: CC(C)(C(=O)Nc1ccc(C#N)c(C(F)(F)F)c1)n1cc(C2CCN(C3CN(C4CN(c5ccc6c(c5)C(=O)N(C5CC(=O)NC5=O)C6=O)C4)C3)CC2)cn1] |
| 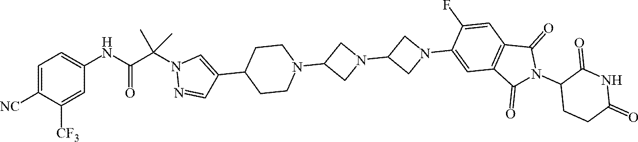 [SMILES: CC(C)(C(=O)Nc1ccc(C#N)c(C(F)(F)F)c1)n1cc(C2CCN(C3CN(C4CN(c5cc6c(cc5F)C(=O)N(C5CCC(=O)NC5=O)C6=O)C4)C3)CC2)cn1] | 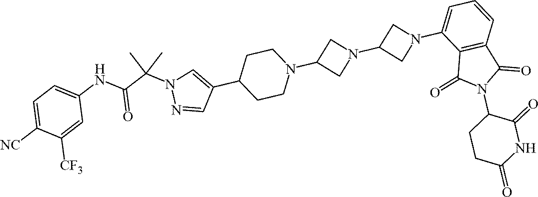 [SMILES: CC(C)(C(=O)Nc1ccc(C#N)c(C(F)(F)F)c1)n1cc(C2CCN(C3CN(C4CN(c5cccc6c5C(=O)N(C5CCC(=O)NC5=O)C6=O)C4)C3)CC2)cn1] |
| 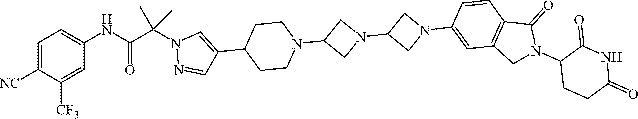 [SMILES: CC(C)(C(=O)Nc1ccc(C#N)c(C(F)(F)F)c1)n1cc(C2CCN(C3CN(C4CN(c5ccc6c(c5)CN(C5CCC(=O)NC5=O)C6=O)C4)C3)CC2)cn1] | 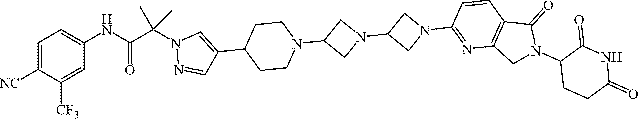 [SMILES: CC(C)(C(=O)Nc1ccc(C#N)c(C(F)(F)F)c1)n1cc(C2CCN(C3CN(C4CN(c5ccc6c(n5)CN(C5CCC(=O)NC5=O)C6=O)C4)C3)CC2)cn1] |
| 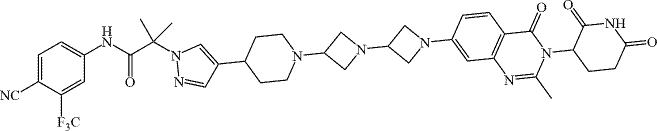 [SMILES: Cc1nc2cc(N3CC(N4CC(N5CCC(c6cnn(C(C)(C)C(=O)Nc7ccc(C#N)c(C(F)(F)F)c7)c6)CC5)C4)C3)ccc2c(=O)n1C1CCC(=O)NC1=O] | 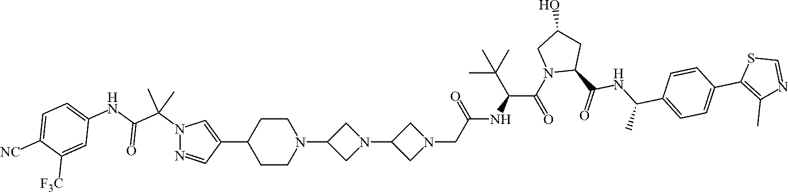 |
| 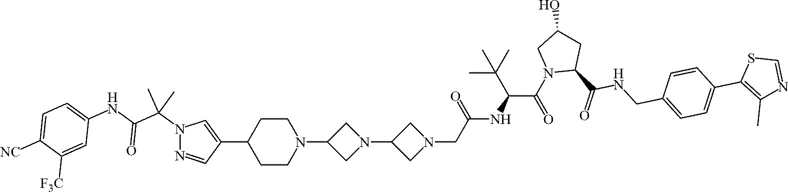 | 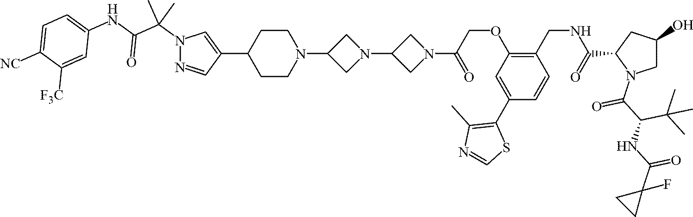 |
| 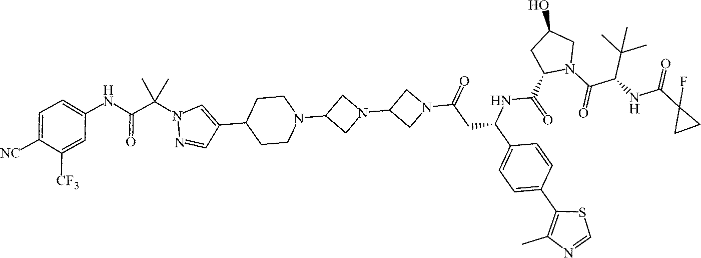 | 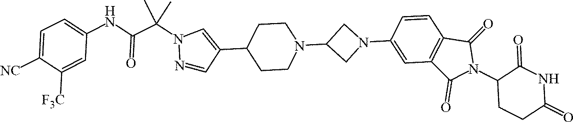 |
| 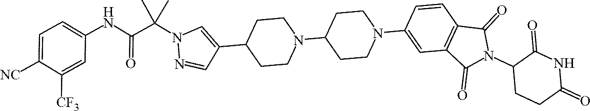 | 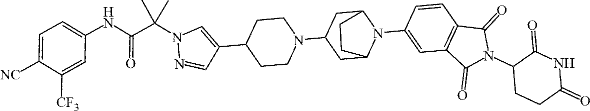 |
| 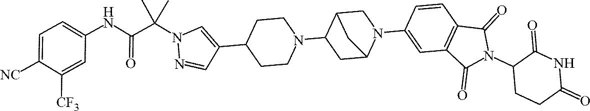 | 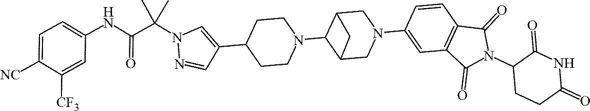 |
| 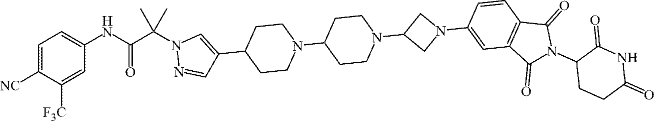 | 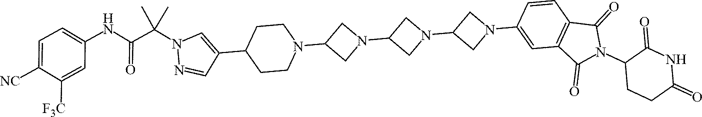 |
| 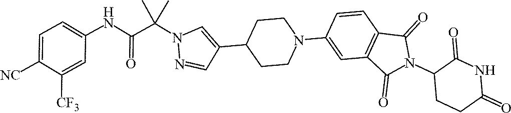 | 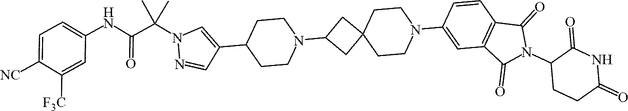 |
| 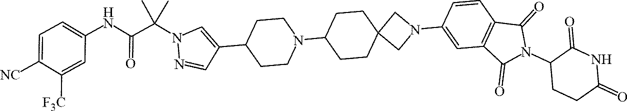 | 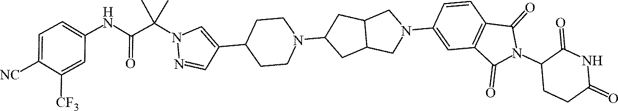 |
| 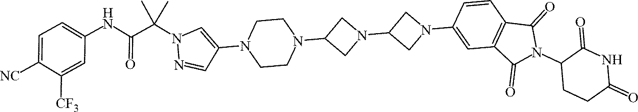 | 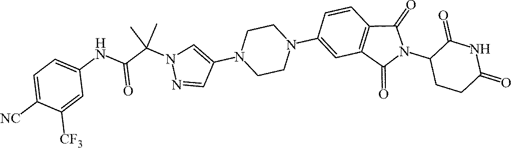 |
| 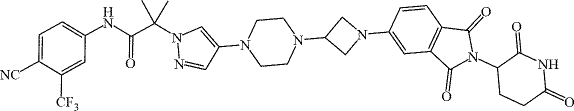 | 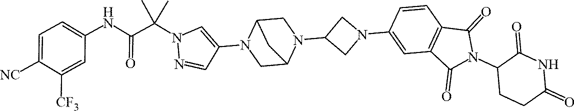 |
| 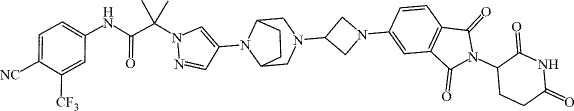 | 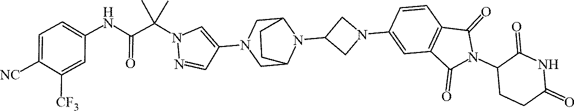 |
| 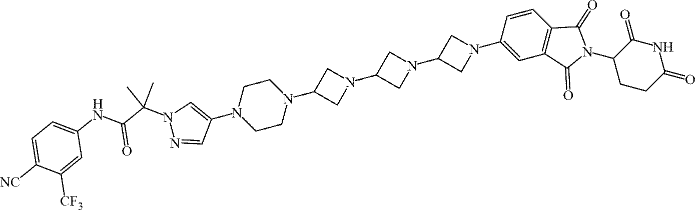 | 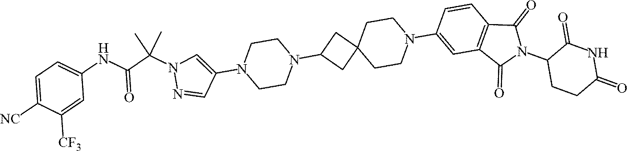 |
| 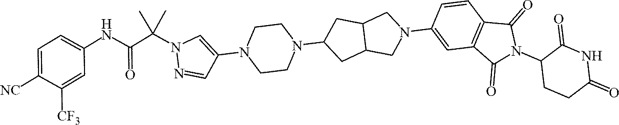 | 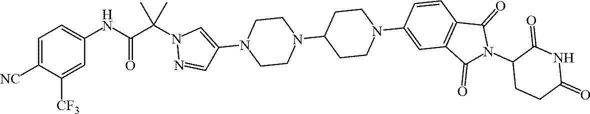 |
| 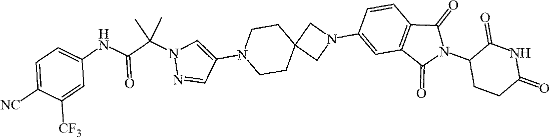 | 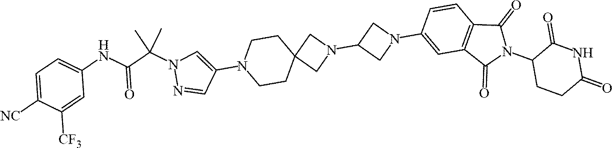 |
| 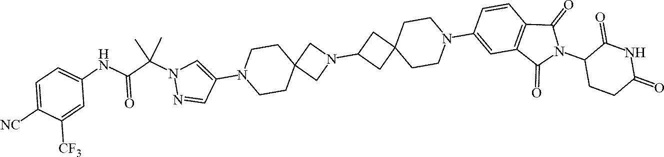 | 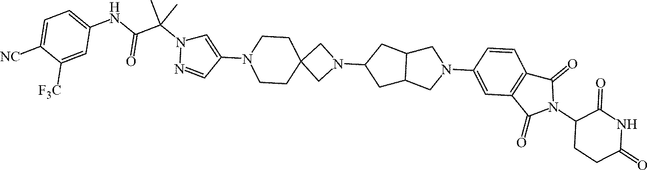 |
| 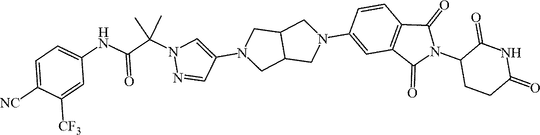 | 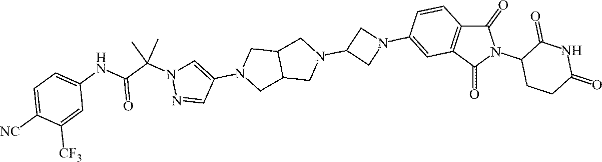 |
| 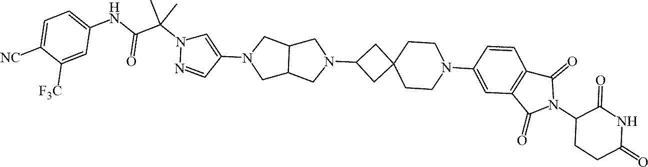 | 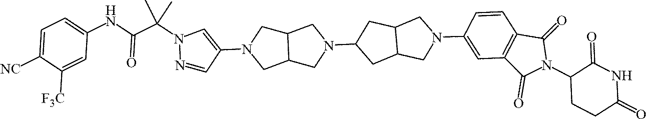 |
| 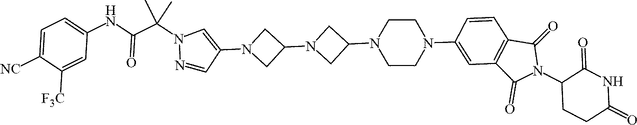 | 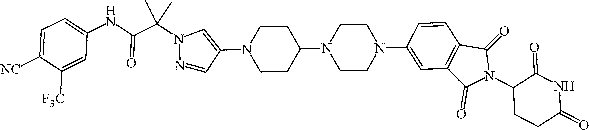 |
| 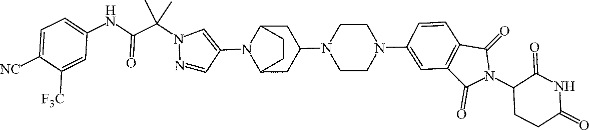 | 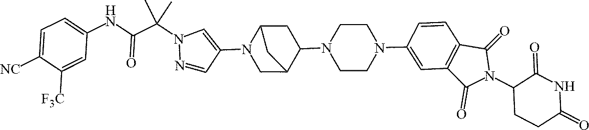 |
| 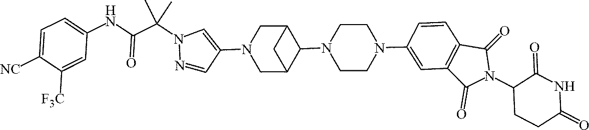 | 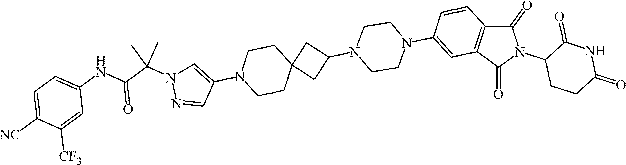 |
| 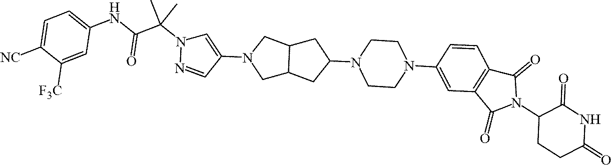 | 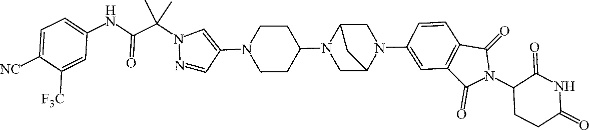 |
| 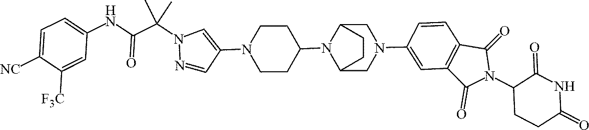 | 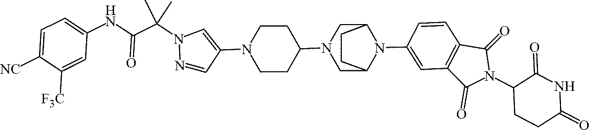 |
| 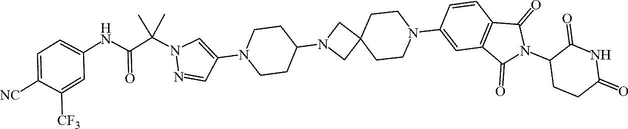 | 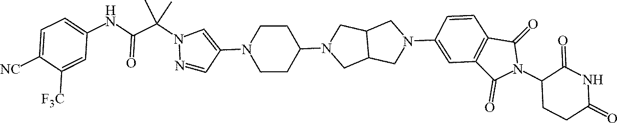 |
| 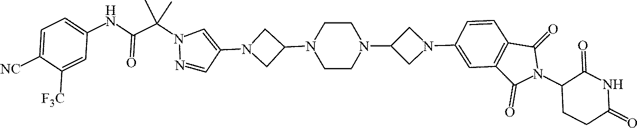 | 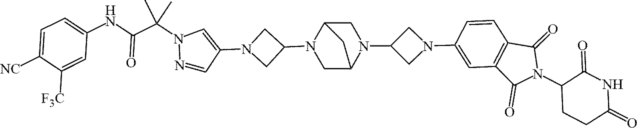 |
| 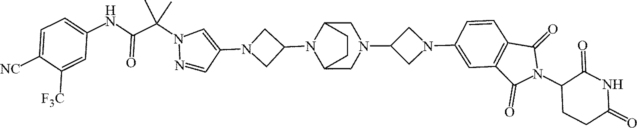 | 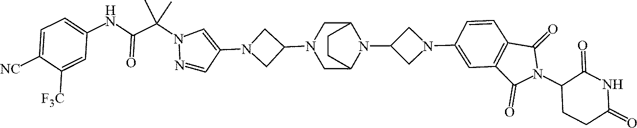 |
| 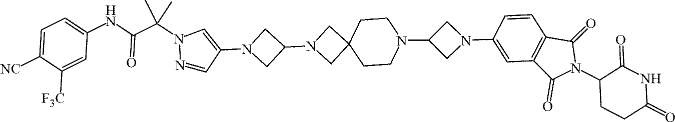 | 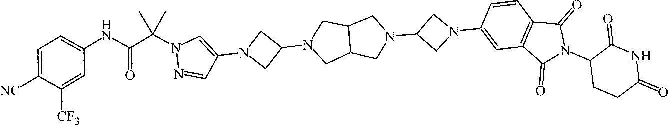 |
| 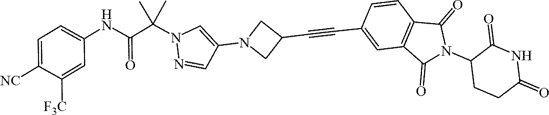 | 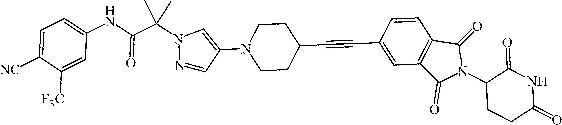 |
| 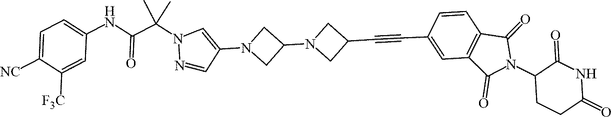 | 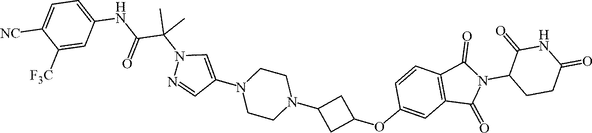 |
| 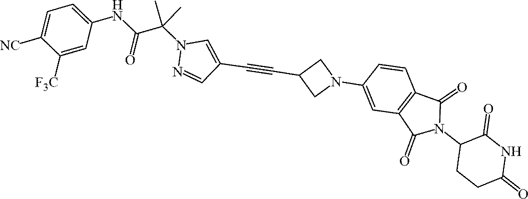 | 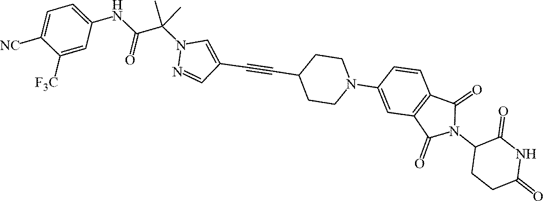 |
| 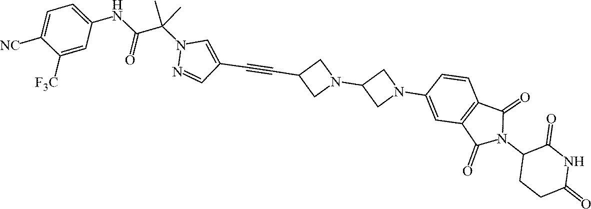 | 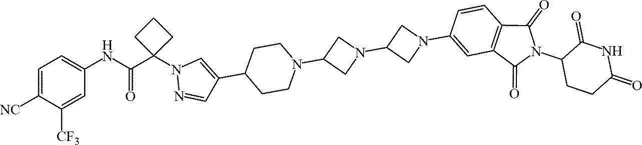 |
| 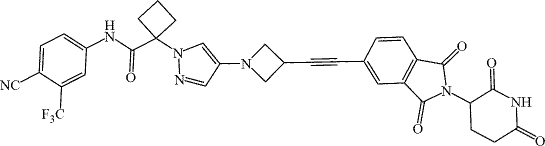 | 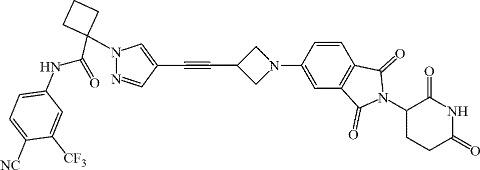 [SMILES: N#Cc1ccc(NC(=O)C2(n3cc(C#CC4CN(c5ccc6c(c5)C(=O)N(C5CCC(=O)NC5=O)C6=O)C4)cn3)CCC2)cc1C(F)(F)F] |
| 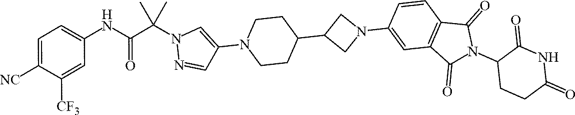 [SMILES: CC(C)(C(=O)Nc1ccc(C#N)c(C(F)(F)F)c1)n1cc(N2CCC(C3CN(c4ccc5c(c4)C(=O)N(C4CCC(=O)NC4=O)C5=O)C3)CC2)cn1] | 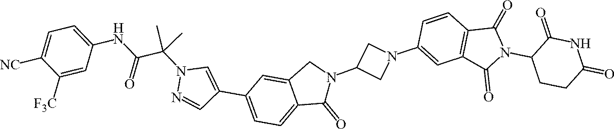 [SMILES: CC(C)(C(=O)Nc1ccc(C#N)c(C(F)(F)F)c1)n1cc(-c2ccc3c(c2)CN(C2CN(c4ccc5c(c4)C(=O)N(C4CCC(=O)NC4=O)C5=O)C2)C3=O)cn1] |
| 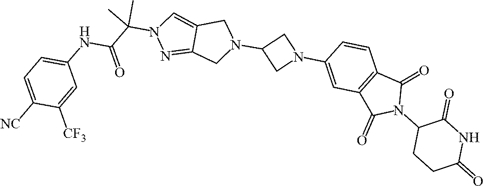 [SMILES: CC(C)(C(=O)Nc1ccc(C#N)c(C(F)(F)F)c1)n1cc2c(n1)CN(C1CN(c3ccc4c(c3)C(=O)N(C3CCC(=O)NC3=O)C4=O)C1)C2] | 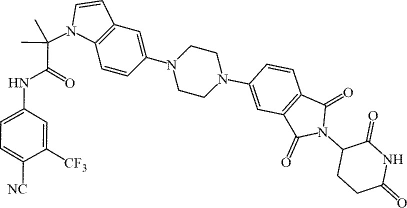 |
| 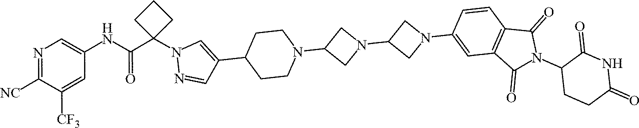 [SMILES: N#Cc1ncc(NC(=O)C2(n3cc(C4CCN(C5CN(C6CN(c7ccc8c(c7)C(=O)N(C7CCC(=O)NC7=O)C8=O)C6)C5)CC4)cn3)CCC2)cc1C(F)(F)F] | 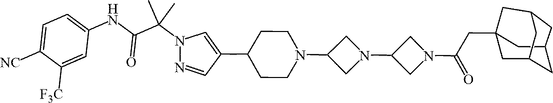 [SMILES: CC(C)(C(=O)Nc1ccc(C#N)c(C(F)(F)F)c1)n1cc(C2CCN(C3CN(C4CN(C(=O)CC56CC7CC(CC(C7)C5)C6)C4)C3)CC2)cn1] |
| 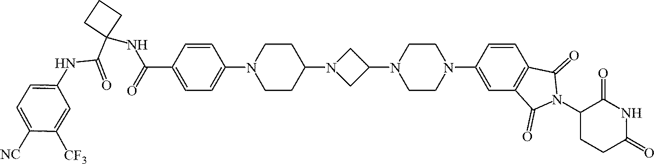 [SMILES: N#Cc1ccc(NC(=O)C2(NC(=O)c3ccc(N4CCC(N5CC(N6CCN(c7ccc8c(c7)C(=O)N(C7CCC(=O)NC7=O)C8=O)CC6)C5)CC4)cc3)CCC2)cc1C(F)(F)F] | 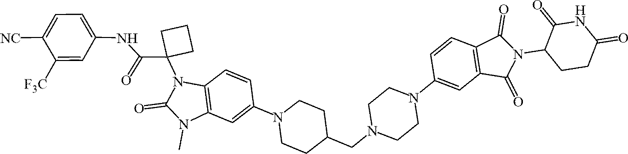 [SMILES: Cn1c(=O)n(C2(C(=O)Nc3ccc(C#N)c(C(F)(F)F)c3)CCC2)c2ccc(N3CCC(CN4CCN(c5ccc6c(c5)C(=O)N(C5CCC(=O)NC5=O)C6=O)CC4)CC3)cc21] |
| 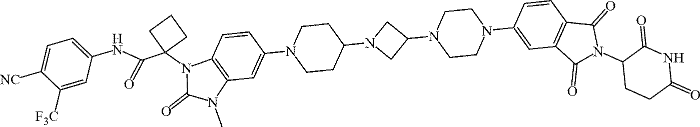 [SMILES: Cn1c(=O)n(C2(C(=O)Nc3ccc(C#N)c(C(F)(F)F)c3)CCC2)c2ccc(N3CCC(N4CC(N5CCN(c6ccc7c(c6)C(=O)N(C6CCC(=O)NC6=O)C7=O)CC5)C4)CC3)cc21] | 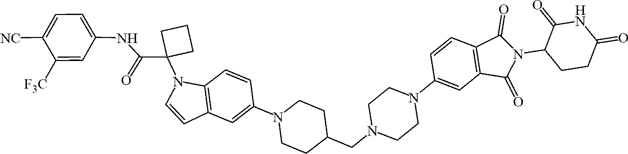 [SMILES: N#Cc1ccc(NC(=O)C2(n3ccc4cc(N5CCC(CN6CCN(c7ccc8c(c7)C(=O)N(C7CCC(=O)NC7=O)C8=O)CC6)CC5)ccc43)CCC2)cc1C(F)(F)F] |
| 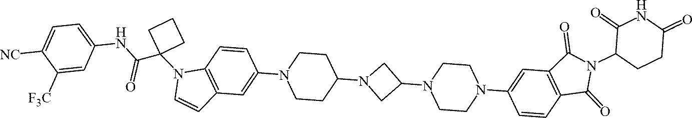 [SMILES: N#Cc1ccc(NC(=O)C2(n3ccc4cc(N5CCC(N6CC(N7CCN(c8ccc9c(c8)C(=O)N(C8CCC(=O)NC8=O)C9=O)CC7)C6)CC5)ccc43)CCC2)cc1C(F)(F)F] | 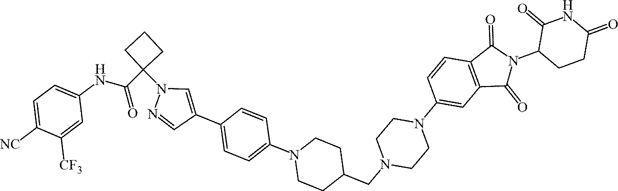 [SMILES: N#Cc1ccc(NC(=O)C2(n3cc(-c4ccc(N5CCC(CN6CCN(c7ccc8c(c7)C(=O)N(C7CCC(=O)NC7=O)C8=O)CC6)CC5)cc4)cn3)CCC2)cc1C(F)(F)F] |
| 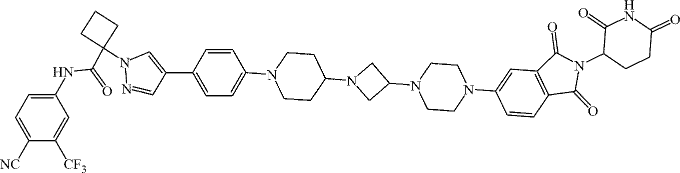 [SMILES: N#Cc1ccc(NC(=O)C2(n3cc(-c4ccc(N5CCC(N6CC(N7CCN(c8ccc9c(c8)C(=O)N(C8CCC(=O)NC8=O)C9=O)CC7)C6)CC5)cc4)cn3)CCC2)cc1C(F)(F)F] | 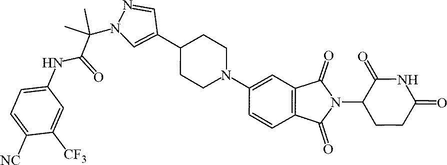 [SMILES: CC(C)(C(=O)Nc1ccc(C#N)c(C(F)(F)F)c1)n1cc(C2CCN(c3ccc4c(c3)C(=O)N(C3CCC(=O)NC3=O)C4=O)CC2)cn1] |
| 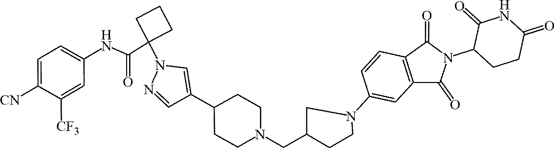 [SMILES: N#Cc1ccc(NC(=O)C2(n3cc(C4CCN(CC5CCN(c6ccc7c(c6)C(=O)N(C6CCC(=O)NC6=O)C7=O)C5)CC4)cn3)CCC2)cc1C(F)(F)F] | 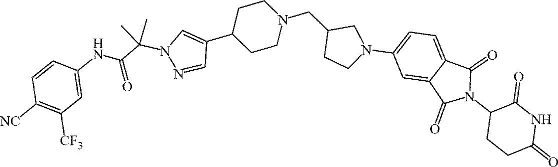 [SMILES: CC(C)(C(=O)Nc1ccc(C#N)c(C(F)(F)F)c1)n1cc(C2CCN(CC3CCN(c4ccc5c(c4)C(=O)N(C4CCC(=O)NC4=O)C5=O)C3)CC2)cn1] |
| 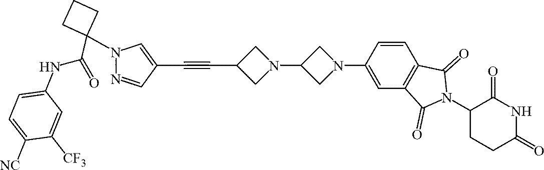 [SMILES: N#Cc1ccc(NC(=O)C2(n3cc(C#CC4CN(C5CN(c6ccc7c(c6)C(=O)N(C6CCC(=O)NC6=O)C7=O)C5)C4)cn3)CCC2)cc1C(F)(F)F] | 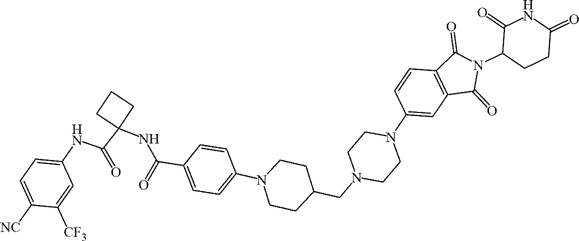 [SMILES: N#Cc1ccc(NC(=O)C2(NC(=O)c3ccc(N4CCC(CN5CCN(c6ccc7c(c6)C(=O)N(C6CCC(=O)NC6=O)C7=O)CC5)CC4)cc3)CCC2)cc1C(F)(F)F] |

The present disclosure relates to a pharmaceutical composition, comprising the above-mentioned compound in the present disclosure or the stereoisomer, deuterated compound, solvate, prodrug, metabolite, pharmaceutically acceptable salt or co-crystal thereof, and a pharmaceutically acceptable carrier.

The present disclosure relates to the use of the above-mentioned compound in the present disclosure or the stereoisomer, deuterated compound, solvate, prodrug, metabolite, pharmaceutically acceptable salt or co-crystal thereof in the preparation of a medicament for treating a disease related to AR activity or expression level.

The present disclosure relates to the use of the above-mentioned compound in the present disclosure or the stereoisomer, deuterated compound, solvate, prodrug, metabolite, pharmaceutically acceptable salt or co-crystal thereof in the preparation of a medicament for treating a disease related to the inhibition or degradation of AR.

The present disclosure relates to the use of the above-mentioned compound in the present disclosure or the stereoisomer, deuterated compound, solvate, prodrug, metabolite, pharmaceutically acceptable salt or co-crystal thereof in the preparation of a medicament for treating a disease related to the activity or expression level of an AR or AR splice variant.

The present disclosure relates to the use of the above-mentioned compound in the present disclosure or the stereoisomer, deuterated compound, solvate, prodrug, metabolite, pharmaceutically acceptable salt or co-crystal thereof in the preparation of a medicament for treating a disease related to the inhibition or degradation of an AR or AR splice variant.

The present disclosure relates to the use of the above-mentioned compound in the present disclosure or the stereoisomer, deuterated compound, solvate, prodrug, metabolite, pharmaceutically acceptable salt or co-crystal thereof, wherein the disease is selected from prostate cancer.

Synthetic method:

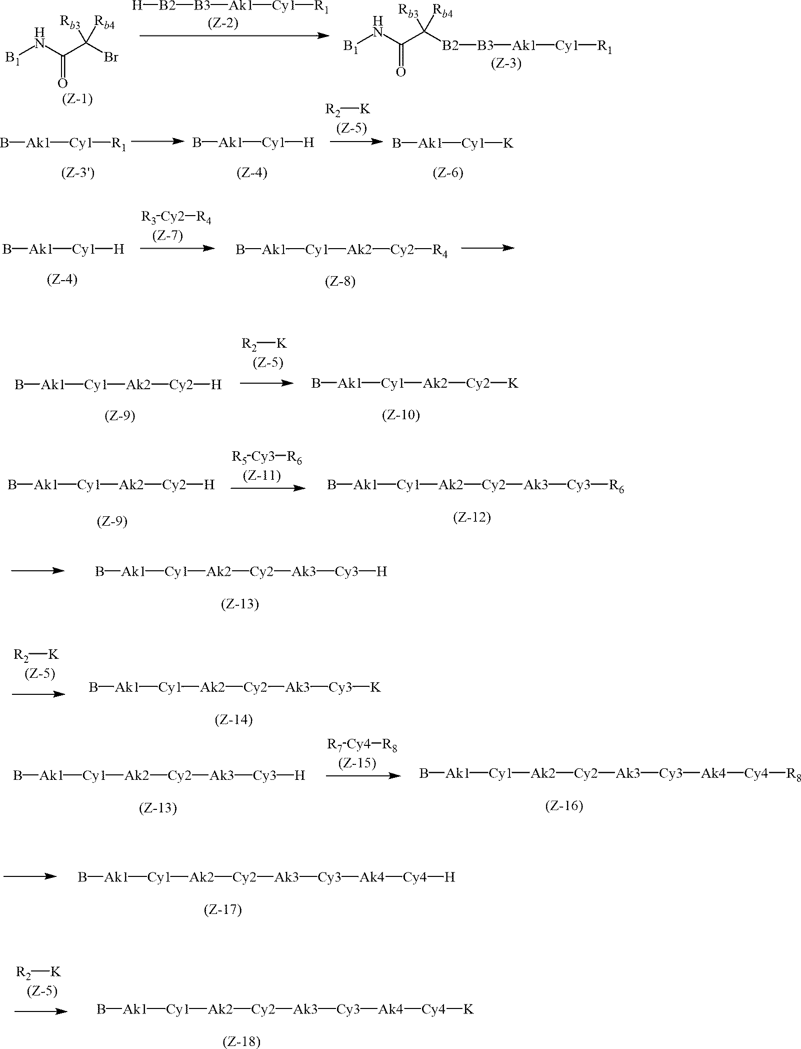

general formula (Z-3), i.e., general formula (Z-3′), is generated by a nucleophilic substitution reaction of general formula (Z-1) with general formula (Z-2), and when R₁ is an amino protecting group, general formula (Z-3′) is deprotected to obtain corresponding general formula (Z-4), and then general formula (Z-6), i.e., general formula (I), is obtained by a nucleophilic substitution reaction of general formula (Z-4) with general formula (Z-5); general formula (Z-8) is generated by a nucleophilic substitution or reductive amination reaction of general formula (Z-4) with general formula (Z-7), and when R₄ is an amino protecting group, general formula (Z-8) is deprotected to obtain corresponding general formula (Z-9), and then general formula (Z-10), i.e., general formula (I), is obtained by a nucleophilic substitution reaction of general formula (Z-9) with general formula (Z-5);
general formula (Z-12) is generated by a nucleophilic substitution or reductive amination reaction of general formula (Z-9) with general formula (Z-11), and when R₆ is an amino protecting group, general formula (Z-12) is deprotected to obtain corresponding general formula (Z-13), and then general formula (Z-14), i.e., general formula (I), is obtained by a nucleophilic substitution reaction of general formula (Z-13) with general formula (Z-5); and
general formula (Z-16) is generated by a nucleophilic substitution or reductive amination reaction of general formula (Z-13) with general formula (Z-15), and when R₈ is an amino protecting group, general formula (Z-16) is deprotected to obtain corresponding general formula (Z-17), and then general formula (Z-18), i.e., general formula (I), is obtained by a nucleophilic substitution reaction of general formula (Z-17) with general formula (Z-5).

The synthetic method of general formula (Z-5) is described in WO 2017197056;
R₁ is selected from H, (═O), —CHO, F, Cl, Br, I or an amino protecting group (preferably Boc);
R₂ is selected from NH₂, F, Cl, Br, I, OTf and OH; and
R₃, R₄, R₅, R₆, R₇ and R₈ are each independently selected from H, (═O), —CHO, H, F, Cl, Br, I, OTf or an amino protecting group (preferably Boc).

Unless stated to the contrary, the terms used in the description and claims have the following meanings.

The carbon, hydrogen, oxygen, sulfur, nitrogen or F, Cl, Br, I involved in the groups and compounds of the present disclosure all comprise their isotopes, and the carbon, hydrogen, oxygen, sulfur or nitrogen involved in the groups and compounds of the present disclosure is optionally further substituted with one or more of their corresponding isotopes, wherein the isotopes of carbon comprise ¹²C, ¹³C and ¹⁴C, the isotopes of hydrogen comprise protium (H), deuterium (D, also known as heavy hydrogen), tritium (T, also known as superheavy hydrogen), the isotopes of oxygen comprise ¹⁶O, ¹⁷O and ¹⁸O, the isotopes of sulfur comprise ³²S, ³³S, ³⁴S and ³⁶S, the isotopes of nitrogen comprise ¹⁴N and ¹⁵N, the isotopes of fluorine comprise ¹⁷F and ¹⁹F, the isotopes of chlorine comprise ³⁵Cl and ³⁷Cl, and the isotopes of bromine comprise ⁷⁹Br and ⁸¹Br.

“Alkyl” refers to a straight or branched saturated aliphatic hydrocarbon group containing 1 to 20 carbon atoms, preferably alkyl containing 1 to 8 carbon atoms, more preferably alkyl containing 1 to 6 carbon atoms, further preferably alkyl containing 1 to 4 carbon atoms. Non-limiting examples include methyl, ethyl, n-propyl, isopropyl, n-butyl, sec-butyl, neobutyl, tert-butyl, n-pentyl, isoamyl, neopentyl, n-hexyl and various branched isomers thereof; the alkyl can be optionally further substituted with 0 to 6 substituents selected from F, Cl, Br, I, hydroxyl, sulfhydryl, nitro, cyano, amino, alkylamino, amido, alkenyl, alkynyl, C₁₋₆ alkyl, C₁₋₆ hydroxyalkyl, C₁₋₆ alkoxy, 3- to 8-membered carbocyclyl, a 3- to 8-membered heterocyclic group, 3- to 8-membered carbocyclyloxy, 3- to 8-membered heterocyclyloxy, carboxyl or a carboxylate group, and the definition of the alkyl described herein is consistent with this definition.

“Alkoxy” refers to -O-alkyl. Non-limiting examples include methoxy, ethoxy, n-propoxy, isopropoxy, n-butoxy, sec-butoxy, tert-butoxy, n-pentoxy, n-hexyloxy, cyclopropoxy and cyclobutoxy. The alkoxy can be optionally further substituted with 0 to 5 substituents selected from F, Cl, Br, I, hydroxyl, sulfhydryl, nitro, cyano, amino, alkylamino, alkenyl, alkynyl, alkyl, hydroxyalkyl, alkoxy, carbocyclyl, a heterocyclic group, carbocyclyloxy, heterocyclyloxy, carboxyl or a carboxylate group. The definition of the alkoxy described herein is consistent with this definition.

“Cycloalkyl” refers to a straight or branched saturated cyclic aliphatic hydrocarbon group containing 3 to 20 carbon atoms, preferably cycloalkyl containing 3 to 10 carbon atoms. Non-limiting examples include cyclopropyl, cyclobutyl, cyclopentyl, cyclohexyl and cycloheptyl. The cycloalkyl can be optionally further substituted with 0 to 5 substituents selected from F, Cl, Br, I, hydroxyl, sulfhydryl, nitro, cyano, amino, alkylamino, alkenyl, alkynyl, alkyl, hydroxyalkyl, alkoxy, carbocyclyl, a heterocyclic group, carbocyclyloxy, heterocyclyloxy, carboxyl or a carboxylate group. The definition of the cycloalkyl described herein is consistent with this definition.

“Heterocyclic group” or “heterocyclic ring” refers to a substituted or unsubstituted saturated or unsaturated aromatic ring or non-aromatic ring, and the aromatic ring or non-aromatic ring can be a 3- to 8-membered monocyclic ring, a 4- to 12-membered bicyclic ring or a 10- to 15-membered tricyclic ring and contains 1 to 3 heteroatoms selected from N, O or S, preferably a 3- to 8-membered heterocyclic group, and the optionally substituted N and S in the ring of the heterocyclic group can be oxidized into various oxidation states. Heterocyclic group can be connected to a heteroatom or carbon atom, and heterocyclic group can be connected to a bridged ring or spiro ring. Non-limiting examples include oxiranyl, azacyclopropyl, oxetanyl, azetidinyl, 1,3-dioxolane, 1,4-dioxolane, 1,3-dioxane, azacycloheptyl, pyridyl, furanyl, thienyl, pyranyl, N-alkylpyrrolyl, pyrimidinyl, pyrazinyl, pyridazinyl, imidazolyl, piperidyl, morpholinyl, thiomorpholinyl, 1,3-dithianyl, dihydrofuranyl, dihydropyranyl, dithiolanyl, tetrahydrofuranyl, tetrahydropyrrolyl, tetrahydroimidazolyl, tetrahydrothiazolyl, tetrahydropyranyl, benzimidazolyl, benzopyridyl, pyrrolopyridyl, benzodihydrofuranyl, azabicyclo[3.2.1]octyl, azabicyclo[5.2.0]nonyl, oxatricyclo[5.3.1.1]dodecyl, azaadamantyl and oxaspiro[3.3]heptyl. The heterocyclic group can be optionally further substituted with 0 to 5 substituents selected from F, Cl, Br, I, ═O, hydroxyl, sulfhydryl, nitro, cyano, amino, alkylamino, amido, alkenyl, alkynyl, alkyl, hydroxyalkyl, alkoxy, carbocyclyl, a heterocyclic group, carbocyclyloxy, heterocyclyloxy, carboxyl or a carboxylate group. The definition of the heterocyclic group described herein is consistent with this definition.

“Spiro ring” refers to a 5- to 20-membered polycyclic group sharing one carbon atom (referred to as a spiro atom) between substituted or unsubstituted monocyclic rings, which may contain 0 to 5 double bonds, and may contain 0 to 5 heteroatoms selected from N, O or S(═O)ₙ. The spiro ring is preferably 6- to 14-membered, further preferably 6- to 12-membered, and more preferably 6- to 10-membered. Its non-limiting examples include:

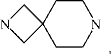

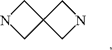

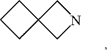

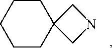

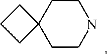

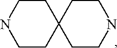

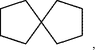

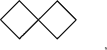

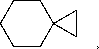

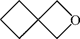

and

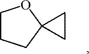

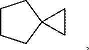

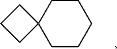

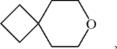

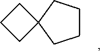

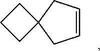

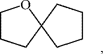

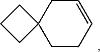

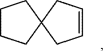

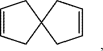

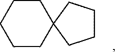

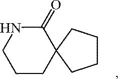

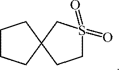

When a substitution occurs, substituents can be 1 to 5 groups selected from F, Cl, Br, I, alkyl, cycloalkyl, alkoxy, haloalkyl, mercaptan, hydroxyl, nitro, sulfhydryl, amino, cyano, isocyano, aryl, heteroaryl, a heterocyclic group, a bridged ring group, a spiro ring group, a fused ring group, hydroxyalkyl, =O, carbonyl, aldehyde, carboxylic acid, formate, —(CH₂)ₘ—C(═O)—R^{a}, —O—(CH₂)ₘ—C(═O)—R^{a}, —(CH₂)ₘ—C(═O)—NR^{b}R^{c}, —(CH₂)ₘS(═O)ₙR^{a}, —(CH₂)ₘ—alkenyl-R^{a}, OR^{d} or —(CH₂)ₘ—alkynyl-R^{a} (wherein m and n are 0, 1 or 2), arylthio, thiocarbonyl, silyl or —NR^{b}R^{c} and the like, wherein R^{b} and R^{c} are independently selected from H, hydroxyl, amino, carbonyl, alkyl, alkoxy, cycloalkyl, a heterocyclic group, aryl, heteroaryl, sulfonyl, trifluoromethylsulfonyl. Alternatively, R^{b} and R^{c} may form five- or six-membered cycloalkyl or a heterocyclic group. R^{a} and R^{d} are each independently selected from aryl, heteroaryl, alkyl, alkoxy, cycloalkyl, a heterocyclic group, carbonyl, an ester group, a bridged ring group, a spiro ring group or a fused ring group. The definition of the spiro ring described herein is consistent with this definition.

“Fused ring” refers to a polycyclic group in which each ring in the system shares an adjacent pair of carbon atoms with other rings in the system, wherein one or more of the rings may contain 0 or more double bonds, which may be substituted or unsubstituted, and each ring in the fused ring system may contain 0 to 5 heteroatoms selected from N, S(═O)ₙ or O (n is selected from 0, 1 or 2). The fused ring is preferably 5- to 20-membered, further preferably 5- to 14-membered, more preferably 5- to 12-membered, and still further preferably 5- to 10-membered. Non-limiting examples include:

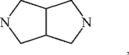

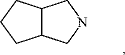

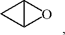

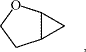

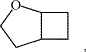

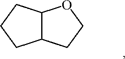

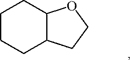

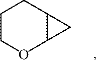

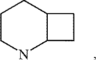

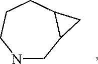

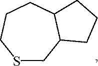

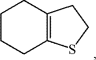

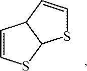

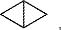

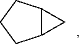

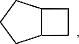

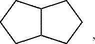

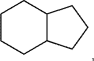

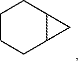

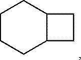

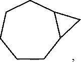

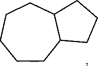

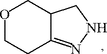

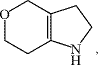

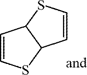

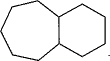

When a substitution occurs, substituents can be 1 to 5 groups selected from F, Cl, Br, I, alkyl, cycloalkyl, alkoxy, haloalkyl, mercaptan, hydroxyl, nitro, sulfhydryl, amino, cyano, isocyano, aryl, heteroaryl, a heterocyclic group, a bridged ring group, a spiro ring group, a fused ring group, hydroxyalkyl, ═O, carbonyl, aldehyde, carboxylic acid, formate, —(CH₂)ₘC(═O)—R^{a}, —O—(CH₂)ₘC(═O)—R^{a}, —(CH₂)ₘC(═O)—NR^{b}R^{c}, —(CH₂)ₘS(═O)ₙR^{a}, —(CH₂)ₘ—alkenyl-R^{a}, OR^{d} or —(CH₂)ₘ—alkynyl-R^{a} (wherein m and n are 0, 1 or 2), arylthio, thiocarbonyl, silyl or —NR^{b}R^{c} and the like, wherein R^{b} and R^{c} are independently selected from H, hydroxyl, amino, carbonyl, alkyl, alkoxy, cycloalkyl, a heterocyclic group, aryl, heteroaryl, sulfonyl, trifluoromethylsulfonyl. Alternatively, R^{b} and R^{c} may form five- or six-membered cycloalkyl or a heterocyclic group. R^{a} and R^{d} are each independently selected from aryl, heteroaryl, alkyl, alkoxy, cycloalkyl, a heterocyclic group, carbonyl, an ester group, a bridged ring group, a spiro ring group or a fused ring group. The definition of the fused ring described herein is consistent with this definition.

“Bridged ring” refers to a polycyclic group containing any two carbon atoms that are not directly connected, which group may contain 0 or more double bonds and can be substituted or unsubstituted, and any ring in the fused ring system may contain 0 to 5 heteroatoms or groups selected from N, S(═O)ₙ or O (wherein n is 0, 1, or 2). The ring atoms contain 5 to 20 atoms, preferably 5 to 14 atoms, further preferably 5 to 12 atoms, and still further preferably 5 to 10 atoms. Non-limiting examples include

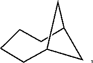

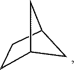

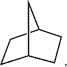

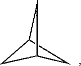

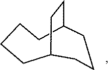

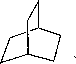

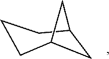

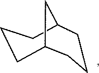

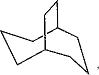

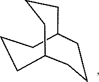

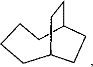

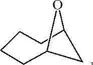

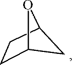

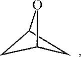

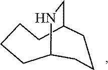

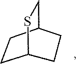

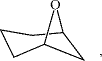

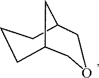

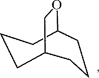

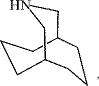

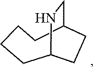

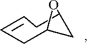

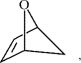

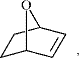

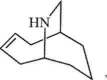

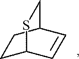

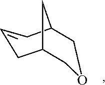

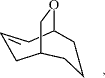

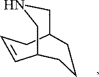

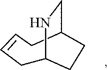

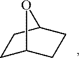

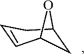

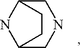

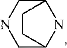

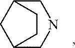

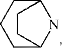

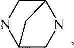

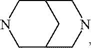

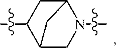

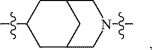

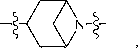

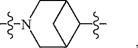

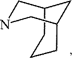

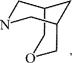

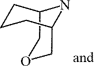

adamantane. When a substitution occurs, substituents can be 1 to 5 groups selected from F, Cl, Br, I, alkyl, cycloalkyl, alkoxy, haloalkyl, mercaptan, hydroxyl, nitro, sulfhydryl, amino, cyano, isocyano, aryl, heteroaryl, a heterocyclic group, a bridged ring group, a spiro ring group, a fused ring group, hydroxyalkyl, =O, carbonyl, aldehyde, carboxylic acid, formate, —(CH₂)ₘ—C(═O)—R^{a}, —O—(CH₂)ₘ—C(═O)—R^{a}, —(CH₂)ₘ—C(═O)—NR^{b}R^{c}, —(CH₂)ₘS(═O)ₙR^{a}, —(CH₂)ₘ—alkenyl-R^{a}, OR^{d} or —(CH₂)ₘ—alkynyl-R^{a} (wherein m and n are 0, 1 or 2), arylthio, thiocarbonyl, silyl or —NR^{b}R^{c} and the like, wherein R^{b} and R^{c} are independently selected from H, hydroxyl, amino, carbonyl, alkyl, alkoxy, cycloalkyl, a heterocyclic group, aryl, heteroaryl, sulfonyl, trifluoromethylsulfonyl. Alternatively, R^{b} and R^{c} may form five- or six-membered cycloalkyl or a heterocyclic group. R^{a} and R^{d} are each independently selected from aryl, heteroaryl, alkyl, alkoxy, cycloalkyl, a heterocyclic group, carbonyl, an ester group, a bridged ring group, a spiro ring group or a fused ring group. The definition of the bridged ring described herein is consistent with this definition.

“Mono-heterocyclic ring” refers to “heterocyclic group” or “heterocyclic ring” in a monocyclic ring system, and the definition of the mono-heterocyclic ring described herein is consistent with this definition.

“Fused heterocyclic ring” refers to a “fused ring” containing heteroatom(s). The definition of the fused heterocyclic ring described herein is consistent with this definition.

“Spiro-heterocyclic ring” refers to a “spiro ring” containing heteroatom(s). The definition of the spiro-heterocyclic ring described herein is consistent with this definition.

“Bridged-heterocyclic ring” refers to a “bridged ring” containing heteroatom(s). The definition of the bridged-heterocyclic ring described herein is consistent with this definition.

“Heteroaryl” or “heteroaryl ring” refers to a substituted or unsubstituted 5-to 14-membered aromatic ring, and contains 1 to 5 heteroatoms or groups selected from N, O or S(═O)ₙ, preferably 5- to 10-membered aromatic ring, further preferably 5- to 6-membered. Non-limiting examples of heteroaryl include, but are not limited to pyridyl, furanyl, thienyl, pyridinyl, pyranyl, N-alkylpyrrolyl, pyrimidinyl, pyrazinyl, pyridazinyl, imidazolyl, benzimidazole, benzimidazolyl, benzopyridine, pyrrolopyridine and the like. The heteroaryl ring can be fused to aryl, a heterocyclic group or a cycloalkyl ring, wherein the ring connected to the parent structure is a heteroaryl ring, and non-limiting examples include

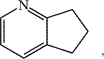

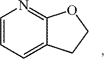

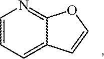

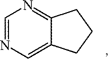

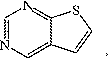

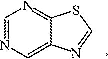

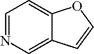

and

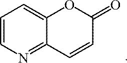

When a substitution occurs, substituents can be 1 to 5 groups selected from F, Cl, Br, I, alkyl, cycloalkyl, alkoxy, haloalkyl, mercaptan, hydroxyl, nitro, sulfhydryl, amino, cyano, isocyano, aryl, heteroaryl, a heterocyclic group, a bridged ring group, a spiro ring group, a fused ring group, hydroxyalkyl, =O, carbonyl, aldehyde, carboxylic acid, formate, —(CH₂)ₘ—C(═O)—R^{a}, —O—(CH₂)ₘ—C(═O)—R^{a}, —(CH₂)ₘ C(═O)—NR^{b}R^{c}, —(CH₂)ₘS(═O)ₙR^{a}, —(CH₂)ₘ—alkenyl-R^{a}, OR^{d}or —(CH₂)ₘ—alkynyl-R^{a} (wherein m and n are 0, 1 or 2), arylthio, thiocarbonyl, silyl or —NR^{b}R^{c} and the like, wherein R^{b} and R^{c} are independently selected from H, hydroxyl, amino, carbonyl, alkyl, alkoxy, cycloalkyl, a heterocyclic group, aryl, heteroaryl, sulfonyl, trifluoromethylsulfonyl. Alternatively, R^{b} and R^{c} may form five- or six-membered cycloalkyl or a heterocyclic group. R^{a} and R^{d} are each independently selected from aryl, heteroaryl, alkyl, alkoxy, cycloalkyl, a heterocyclic group, carbonyl, an ester group, a bridged ring group, a spiro ring group or a fused ring group. The definition of the heteroaryl or heteroaryl ring described herein is consistent with this definition.

“Substituted with 0 to X substituents” refers to substituted with 0, 1, 2, 3 ... X substituents, wherein X is selected from any integer between 1 and 10. For example, “substituted with 0 to 4 substituents” refers to substituted with 0, 1, 2, 3 or 4 substituents. For example, “substituted with 0 to 5 substituents” refers to substituted with 0, 1, 2, 3, 4 or 5 substituents. For example, “bridged-heterocyclic ring is optionally further substituted with 0 to 4 substituents selected from H or F” means that the bridged-heterocyclic ring is optionally further substituted with 0, 1, 2, 3 or 4 substituents selected from H or F.

An X- to Y-membered ring (X is an integer, 3 ≤ X < Y, and Y is selected from any integer between 4 and 12) includes X+1-, X+2-, X+3-, X+4-,...to Y-membered rings. Rings include a heterocyclic ring, a carbocyclic ring, an aromatic ring, aryl, heteroaryl, cycloalkyl, a mono-heterocyclic ring, a fused heterocyclic ring, a spiro-heterocyclic ring or a bridged-heterocyclic ring. For example, a “4- to 7-membered mono-heterocyclic ring” refers to a 4-, 5-, 6- or 7-membered mono-heterocyclic ring, and a “5- to 10-membered fused heterocyclic ring” refers to a 5-, 6-, 7-, 8-, 9- or 10-membered fused heterocyclic ring.

The term “optional” or “optionally” refers to that the events or circumstances subsequently described may but not necessarily occur, and the description includes the occasions where the events or circumstances occur or do not occur. For example, “alkyl optionally substituted with F” means that an alkyl may but not necessarily be substituted with F, and the description includes the case where the alkyl is substituted with F and the case where the alkyl is not substituted with F.

“Pharmaceutically acceptable salt” or “pharmaceutically acceptable salt thereof” refers to a salt of the compound of the present disclosure maintaining the biological effectiveness and characteristics of the free acid or free base, and obtained by reacting the free acid with a non-toxic inorganic base or organic base, and reacting the free base with a non-toxic inorganic acid or organic acid.

“Pharmaceutical composition” refers to a mixture of one or more of the compounds of the present disclosure, a pharmaceutically acceptable salt or a prodrug thereof, and other chemical components, wherein “other chemical components” refer to pharmaceutically acceptable carriers, excipients and/or one or more other therapeutic agents.

“Carrier” refers to a material that does not cause significant irritation to the organism and does not eliminate the biological activity and characteristics of the administered compound.

The term “co-crystal” refers to a crystal formed by the combination of active pharmaceutical ingredient (API) and co-crystal former (CCF) under the action of hydrogen bonds or other non-covalent bonds. The pure state of API and CCF are both solid at room temperature, and there is a fixed stoichiometric ratio between various components. The co-crystal is a multi-component crystal, which includes both a binary co-crystal formed between two neutral solids and a multi-element co-crystal formed between a neutral solid and a salt or solvate.

The term “stereoisomer” refers to an isomer produced as a result of different spatial arrangement of atoms in molecules, including cis-trans isomers, enantiomers and conformational isomers.

“DC₅₀” refers to the dose at which 50% of the protein is degraded.

“IC₅₀” refers to the concentration of a drug or inhibitor required to inhibit half of a given biological process (or a component of the process such as an enzyme, a receptor and a cell).

### DETAILED DESCRIPTION OF EMBODIMENTS

The technical solutions of the present disclosure will be described in detail below in conjunction with examples, but the protection scope of the present disclosure includes but is not limited thereto.

To achieve the objects of the present disclosure, according to organic synthesis techniques known to those skilled in the art, and starting from commercially available chemicals and/or compounds described in chemical documents, the preparation of the compounds, “commercially available chemicals”, for use in the reactions described herein is obtained from standard commercial sources, including Shanghai Aladdin Bio-Chem Technology Co., Ltd., Shanghai Macklin Biochemical Co., Ltd., Sigma-Aldrich, Alfa Aesar (China) Chemical Co., Ltd., Tokyo Chemical Industry (Shanghai) Co., Ltd., Energy Chemical Co., Ltd., Shanghai Titan Scientific Co., Ltd., Kelong Chemical Co., Ltd., J&K Scientific and the like.

References and monographs in the art introduce in detail the synthesis of reactants that can be used to prepare the compounds described herein, or provide articles describing the preparation method for reference. The references and monographs include: “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992; Fuhrhop, J. and Penzlin G. “Organic Synthesis: Concepts, Methods, Starting Materials”, Second, Revised and Enlarged Edition (1994) John Wiley & Sons ISBN: 3 527-29074-5; Hoffman, R.V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modem Carbonyl Chemistry” (2000) Wiley-VCH, ISBN: 3-527-29871-1; Patai, S. “Patai’s 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J.C., “Intermediate Organic Chemistry” 2nd Edition (1993) Wiley-Interscience, ISBN: 0-471-57456-2; “Industrial Organic Chemicals: Starting Materials and Intermediates: An Ullmann’s Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups”, John Wiley & Sons, in 73 volumes.

Specific and similar reactants can be selectively identified by the indexes of known chemicals prepared by the Chemical Abstracts Service of the American Chemical Society, wherein the indexes are available in most public libraries or university libraries and online. Chemicals that are known but not commercially available in the catalog are optionally prepared by custom chemical synthesis plants, wherein many of standard chemical supply plants (for example, those listed above) provide custom synthesis services. Reference document for the preparation and selection of the pharmaceutically acceptable salts of the compounds described herein is P. H. Stahl & C. G. Wermuth “Handbook of Pharmaceutical Salts”, Verlag Helvetica Chimica Acta, Zurich, 2002.

The technical solutions of the present disclosure will be described in detail by the following examples, but the scope of protection of the present disclosure includes but is not limited thereto.

The compounds used in the reactions described herein are prepared according to organic synthesis techniques known to those skilled in the art, and starting from commercially available chemicals and(or) compounds described in chemical documents. “Commercially available chemicals” are obtained from regular commercial sources, and suppliers include: Titan Technology Co., Ltd., Energy Chemical Co., Ltd., Shanghai Demo Co., Ltd., Chengdu Kelong Chemical Co., Ltd., Accela ChemBio Co., Ltd., PharmaBlock Sciences (Nanjing), Inc., WuXi Apptec Co., Ltd., J&K Scientific and the like.

The structures of the compounds are determined by nuclear magnetic resonance (NMR) or (and) mass spectrometry (MS). The NMR shift (δ) is given in the unit of 10⁻⁶ (ppm). NMR is measured with (Bruker Avance III 400 and Bruker Avance 300) NMR instrument, and the solvent for determination is deuterated dimethyl sulfoxide (DMSO-d₆), deuterated chloroform (CDCl₃), deuterated methanol (CD₃OD), and the internal standard is tetramethylsilane (TMS);

MS is measured with (Agilent 6120B(ESI) and Agilent 6120B(APCI));

HPLC is determined with Agilent 1260DAD high pressure liquid chromatography (Zorbax SB-C18 100 × 4.6 mm, 3.5 µM);

Yantai Huanghai HSGF254 or Qingdao GF254 silica gel plate is used as a thin layer chromatography silica plate; the specification of the silica gel plate used for thin layer chromatography (TLC) is 0.15 mm-0.20 mm; and the specification when separating and purifying a product by thin layer chromatography is 0.4 mm - 0.5 mm.

For the column chromatography, Yantai Huanghai silica gel of 200-300 mesh silica gel is generally used as a carrier.

DMSO: dimethyl sulfoxide, DIPEA: N,N′-diisopropylethylamine, DCE: dichloroethane, DCM: dichloromethane, DIPEA: N,N-diisopropylethylamine.

### Synthesis of Intermediate A

### Tert-butyl 4-(1H-pyrazol-4-yl)piperidine-1-carboxylate (intermediate A)

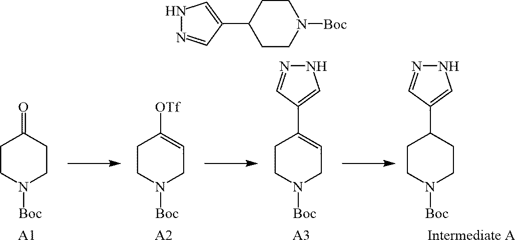

### Step 1: Tert-butyl 4-(trifluoromethylsulfonyloxy)-3,6-dihydro-2H-pyridine-1-carboxylate (A2)

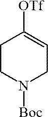 [SMILES: CC(C)(C)OC(=O)N1CC=C(OS(=O)(=O)C(F)(F)F)CC1]

N-Bocpiperidone (A1) (5.21 g, 26.1 mmol) was dissolved in 26 mL of tetrahydrofuran, and then the mixture was cooled to -78° C. under nitrogen atmosphere. A solution of lithium bis(trimethylsilyl)amide (1 mol/L) in tetrahydrofuran (28.5 mL) was slowly added dropwise, and after the addition was completed, the reaction was stirred at -78° C. for 1h. A solution of N-phenylbis(trifluoromethanesulfonyl)imide (10.2 g, 28.6 mmol) in tetrahydrofuran (26 mL) was added dropwise, and after the addition was completed, the reaction solution was naturally warmed to room temperature and reacted for 3 h. The reaction was quenched by dropwise adding 20 mL of saturated sodium bicarbonate solution, and 50 mL of ethyl acetate was added. The liquid separation was conducted, and the organic layer was washed with 20 mL of saturated sodium chloride solution, dried over anhydrous sodium sulfate and concentrated under reduced pressure to obtain a crude product, which was separated and purified by silica gel column chromatography (petroleum ether/ethyl acetate (v/v) = 100/0-9/1) to obtain tert-butyl 4-(trifluoromethylsulfonyloxy)-3,6-dihydro-2H-pyridine-1-carboxylate (A2) (7.80 g, yield: 90%).

### Step 2: Tert-butyl 4-(1H-pyrazol-4-yl)-3,6-dihydro-2H-pyridine-1-carboxylate (A3)

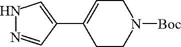 [SMILES: CC(C)(C)OC(=O)N1CC=C(c2cn[nH]c2)CC1]

Tert-butyl 4-(trifluoromethylsulfonyloxy)-3,6-dihydro-2H-pyridine-1-carboxylate (A2) (1.00 g, 3.02 mmol) was dissolved in 5 mL of acetonitrile. 4-(4,4,5,5-tetramethyl-1,3,2-dioxaborolan-2-yl)-1H-pyrazole (0.761 g, 3.92 mmol), 5 mL of saturated sodium bicarbonate aqueous solution and Pd(dppf)Cl₂•CH₂Cl₂ (122 mg, 0.16 mmol) were successively added, and the mixture was reacted at 110° C. for 30 min under a microwave condition. The reaction was cooled to room temperature, and 20 mL of water was added. The mixture was extracted with ethyl acetate (20 mL × 2). The organic layers were combined, washed with 20 mL of saturated sodium chloride solution, dried over anhydrous sodium sulfate and concentrated under reduced pressure to obtain a crude product, which was separated and purified by silica gel column chromatography (petroleum ether/ethyl acetate (v/v) = 4/1-1/1) to obtain tert-butyl 4-(1H-pyrazol-4-yl)-3,6-dihydro-2H-pyridine-1-carboxylate (A3) (0.420 g, yield: 56%).

¹HNMR (400 MHz, CDC1₃) δ 7.61 (s, 2H), 5.92 (s, 1H), 4.07 - 4.00 (m, 2H), 3.62 (t, 2H), 2.46 - 2.37 (m, 2H), 1.48 (s, 9H).

$$\text{LCMS} \text{m} / \text{z} = 250.3 {\left[\text{M+1}\right]}^{+}$$

### Step 3: Tert-butyl 4-(1H-pyrazol-4-yl)piperidine-1-carboxylate (intermediate A)

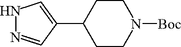 [SMILES: CC(C)(C)OC(=O)N1CCC(c2cn[nH]c2)CC1]

Tert-butyl 4-(1H-pyrazol-4-yl)-3,6-dihydro-2H-pyridine-1-carboxylate (A3) (0.400 g, 1.60 mmol) was dissolved in 10 mL of anhydrous ethanol. 200 mg of 10% palladium on carbon was added, and the mixture was subjected to hydrogen replacement three times and reacted at 40° C. for 5 h under hydrogen atmosphere. The reaction solution was filtered with celite, and the filtrate was concentrated under reduced pressure to obtain tert-butyl 4-(1H-pyrazol-4-yl)piperidine-1-carboxylate (intermediate A) (0.4 g, yield: > 99%).

¹HNMR (400 MHz, CDC1₃) δ 8.94 (s, 1H), 7.43 (s, 2H), 4.30 - 4.00 (m, 2H), 2.83 (t, 2H), 2.74 - 2.63 (m, 1H), 1.96 - 1.84 (m, 2H), 1.60 - 1.39 (m, 11H).

### Synthesis of Intermediate B

### Tert-butyl 3-ethynyl-[1,3′-biazetidine]-1′-carboxylate (intermediate B)

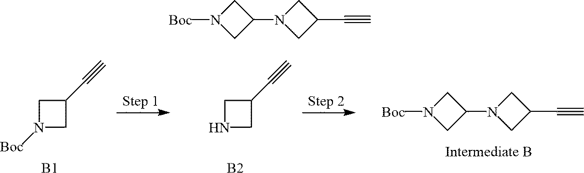

### Step 1: 3-ethynylazetidine (B2) Hydrochloride

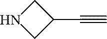

Tert-butyl 3-ethynylazetidine-carboxylate (B1) (2.77 g, 15.28 mmol) was added to a 250 mL single-necked round-bottom flask containing 40 mL of 3 mol/L ethyl acetate hydrochloride solution, and the mixture was stirred at room temperature for 4 h. The reaction solution was concentrated under reduced pressure to obtain crude 3-ethynylazetidine (B2) hydrochloride (1.74 g).

$$\text{LCMS} \text{m} / \text{z} = 82.2 {\left[\text{M+1}\right]}^{+}$$

### Step 2: Tert-butyl 3-ethynyl-[1,3′-biazetidine]-1′-carboxylate (intermediate B)

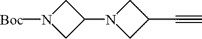 [SMILES: C#CC1CN(C2CN(C(=O)OC(C)(C)C)C2)C1]

The crude 3-ethynylazetidine (B2) hydrochloride (1.74 g) was weighed into a 250 mL single-necked round-bottom flask and dissolved in 100 mL of 1,2-dichloroethane. 3 mL of triethylamine was added, and then tert-butyl 3-oxoazetidine-1-carboxylate (6.33 g, 37.00 mmol), acetic acid (3.11 g, 51.80 mmol) and 6 g ofanhydrous sodium sulfate were successively added. The mixture was heated to 60° C. and reacted for 2 h. The reaction solution was cooled to room temperature, sodium triacetoxyborohydride (18.82 g, 88.79 mmol) was added in portions, and the resulting solution was reacted at room temperature for 16 h. After the reaction was completed, 50 mL of water was added to the reaction solution, and the liquid separation was conducted. The organic phase was washed successively once with 50 mL of saturated sodium bicarbonate solution, 50 mL of water and 50 mL of saturated brine, dried over anhydrous sodium sulfate, filtered and concentrated under reduced pressure. The crude product was separated and purified by silica gel column chromatography (petroleum ether/ethyl acetate (v/v) = 4/1) to obtain tert-butyl 3-ethynyl-[1,3′-biazetidine]-1′-carboxylate (intermediate B) (3.33 g, two-step yield calculated from compound B1: 92%).

$$\text{LCMS} \text{m} / \text{z} = 237.2 {\left[\text{M+1}\right]}^{+}$$

### Synthesis of Intermediate 1

### N-cyano-3-(trifluoromethyl)phenyl)-2-methyl-2-(piperidin-4-yl)-1H-pyrazol-1-yl)propanamide (Intermediate 1)

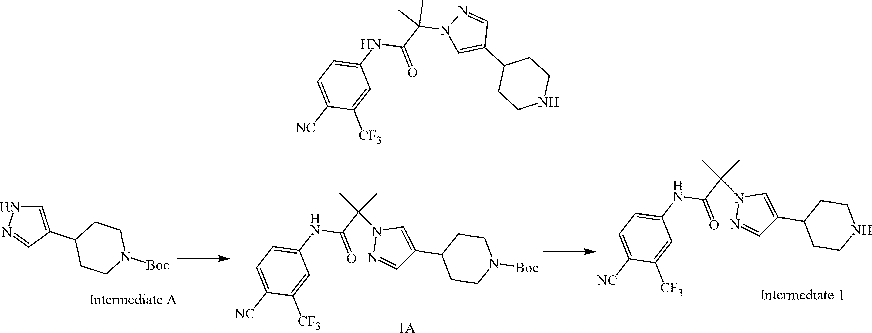

### Step 1: Tert-butyl 4-(1-(1-((4-cyano-3-(trifluoromethyl)phenyl)amino)-2-methyl-1-oxopropan-2-yl)-1H-pyrazol-4-yl)piperidine-1-carboxylate (1A)

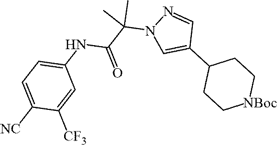

2-bromo-N-(4-cyano-3-(trifluoromethyl)phenyl)-2-methylpropanamide (see WO 2020063407 for the synthetic method) (4.0 g, 11.9 mmol) was dissolved in 50 mL of acetonitrile. Tert-butyl 4-(1H-pyrazol-4-yl)piperidine-1-carboxylate (intermediate A)(2.5 g, 9.9 mmol) and cesium carbonate (6.5 g, 19.9 mmol) were added, and the reaction was stirred at 50° C. for 1 h. The reaction solution was concentratedunder reduced pressure, diluted with 100 mL of ethyl acetate, washed with 100 mL of water, dried over anhydrous sodium sulfate and concentrated under reduced pressure. The crude product was separated and purified by silica gel column chromatography (petroleum ether/ethyl acetate (v/v) = 2 : 1) to obtain tert-butyl 4-(1-(1-((4-cyano-3-(trifluoromethyl)phenyl)amino)-2-methyl-1-oxopropan-2-yl)-1H-pyrazol-4-yl)piperidine-1-carboxylate (1A) (3.7 g, yield: 74%).

### Step 2: N-(4-cyano-3-(trifluoromethyl)phenyl)-2-methyl-2-(4-(piperidin-4-yl)-1H-pyrazol-1-yl)propanamide (Intermediate 1)

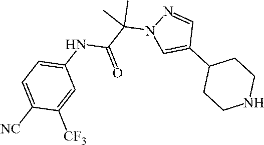

Tert-butyl 4-(1-(1-((4-cyano-3-(trifluoromethyl)phenyl)amino)-2-methyl-1-oxopropan-2-yl)-1H-pyrazol-4-yl)piperidine-1-carboxylate (1A) (3.7 g, 7.3 mmol) was dissolved in 50 mL of DCM. 15 mL of trifluoroacetic acid was added, and the mixture was stirred at room temperature for 3 h. The reaction solution was concentrated under reduced pressure and diluted with 50 mL of dichloromethane. The pH was adjusted to 9.0 with saturated sodium bicarbonate solution. The organic phase was separated, washed with 50 mL of water, dried over anhydrous sodium sulfate and concentrated under reduced pressure to obtain N-(4-cyano-3-(trifluoromethyl)phenyl)-2-methyl-2-(4-(piperidin-4-yl)-1H-pyrazol-1-yl)propanamide (intermediate 1) (2.9 g, yield: 98%).

$$\text{LCMS} \text{m} / \text{z} = 406.3 {\left[\text{M+1}\right]}^{+}$$

### Synthesis of Intermediate 2

### N-cyano-3-(trifluoromethyl)phenyl)-1-(piperidin-4-yl)-1H-pyrazol-1-yl)cyclobutane-1-carboxamide (Intermediate 2)

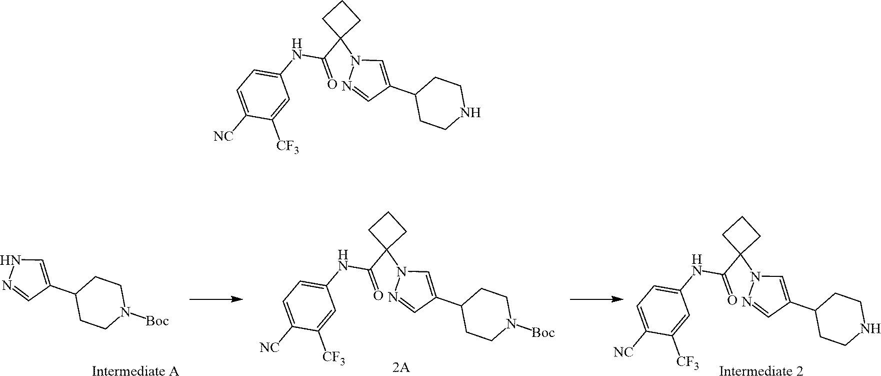

### Step 1: Tert-butyl 4-[1-[1-[[4-cyano-3-(trifluoromethyl)phenyl]carbamoyl]cyclobutyl]pyrazol-4-yl] piperidine-1-carboxylate (2A)

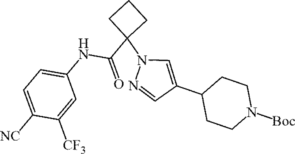

Tert-butyl 4-(1H-pyrazol-4-yl)piperidine-1-carboxylate (intermediate A) (0.145 g, 0.576 mmol) was dissolved in 5 mL of acetonitrile. 1-bromo-N-(4-cyano-3-(trifluoromethyl)phenyl)cyclobutane-1-carboxamide (see WO 2020063407 for the synthetic method) (0.200 g, 0.576 mmol) and cesium carbonate (0.207 g, 0.635 mmol) were added successively, and the reaction was stirred at 80° C. for 3 h. The reaction solution was cooled to room temperature, and 20 mL of ethyl acetate and 10 mL of water were added. The liquid separation was conducted. The organic layer was dried over anhydrous sodium sulfate and concentrated under reduced pressure to obtain a crude product, which was separated and purified by silica gel column chromatography (petroleum ether/ethyl acetate (v/v) = 4 : 1) to obtain tert-butyl 4-[1-[1-[[4-cyano-3-(trifluoromethyl)phenyl]carbamoyl]cyclobutyl]pyrazol-4-yl] piperidine-1-carboxylate (2A) (0.060 g, yield: 20%).

¹HNMR (400 MHz, CDC1₃) δ 9.14 (s, 1H), 8.00 - 7.96 (m, 1H), 7.81 - 7.70 (m, 2H), 7.60 (s, 1H), 7.39 (s, 1H), 4.23 - 4.12 (m, 2H), 3.10 - 2.99 (m, 2H), 2.87 - 2.61 (m, 5H), 2.27 - 2.14 (m, 1H), 2.14 - 2.04 (m, 1H), 1.94 - 1.85 (m, 2H), 1.58 - 1.40 (m, 11H).

### Step 2: N-(4-cyano-3-(trifluoromethyl)phenyl)-1-(4-(piperidin-4-yl)-1H-pyrazol-1-yl)cyclobutane-1-carboxamide (Intermediate 2)

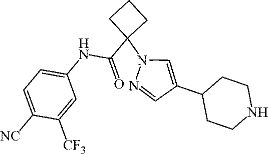

Tert-butyl 4-[1-[1-[[4-cyano-3-(trifluoromethyl)phenyl]carbamoyl]cyclobutyl]pyrazol-4-yl] piperidine-1-carboxylate (2A) (1.50 g, 2.90 mmol) was dissolved in 10 mL of dichloromethane. 4 mL of trifluoroacetic acid was added, and the mixture was reacted at room temperature for 20 min. The reaction solution was concentrated under reduced pressure, 50 mL of dichloromethane was added to the residue, the pH was adjusted to 9 with saturated sodium bicarbonate solution, and the liquid separation was conducted. The organic layer was dried over anhydrous sodium sulfate and concentrated under reduced pressure to obtain N-(4-cyano-3-(trifluoromethyl)phenyl)-1-(4-(piperidin-4-yl)-1H-pyrazol-1-yl)cyclobutane-1-carboxamide (intermediate 2) (1.20 g, yield: > 99%).

$$\text{LCMS} \text{m} / \text{z} = 418.2 {\left[\text{M+1}\right]}^{+}$$

### Example 1

### N-cyano-3-(trifluoromethyl)phenyl)-2-(1-((3aR,6aS)-2-(2-(2,6-dioxopiperidin-3-yl)-1,3-dioxoisoindolin-5-yl)octahydrocyclopenta[c]pyrrol-5-yl)piperidin-4-yl)-1H-pyrazol-1-yl)-2-methylpropanamide (Compound 1)

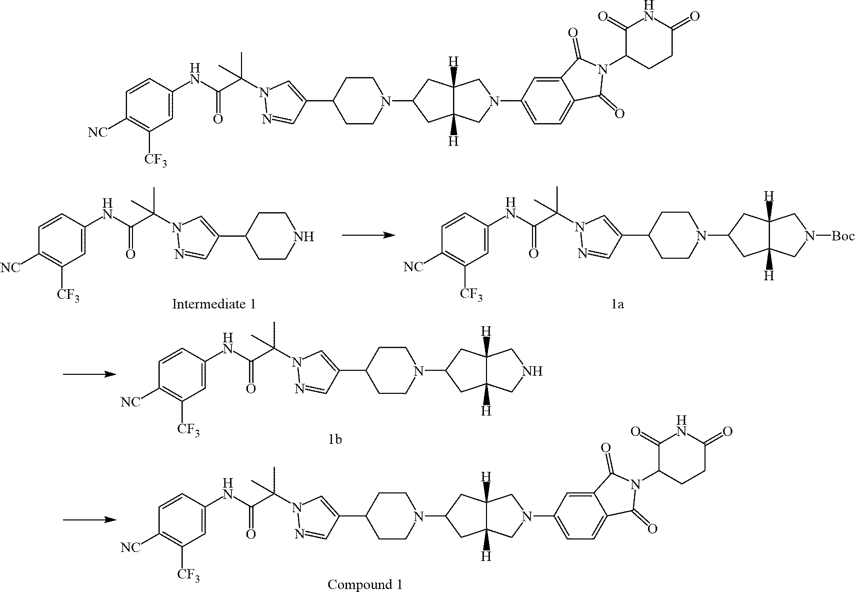

### Step 1: tert-butyl(3aR,6aS)-5-(4-(l-(l-((4-cyano-3-(trifluoromethyl)phenyl)amino)-2-methyl-1-oxopropan-2-yl)-1H-pyrazol-4-yl)piperidin-1-yl)hexahydrocyclopenta[c]pyrrole-2(1H)-carboxylate (1a)

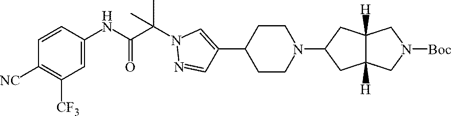

N-cyano-3-(trifluoromethyl)phenyl)-2-methyl-2-(piperidin-4-yl)-1H-pyrazol-1-yl)propanamide (intermediate 1) (202 mg, 0.50 mmol) was dissolved in 15 mL of DCE. (3aR,6aS)-5-oxo hexahydrocyclopenta[c]pyrrole-2(1H)-carboxylate (225 mg, 1.00 mmol) was added, and the mixture was stirred at room temperature for 0.5 h. After that, sodium triacetoxyborohydride (318 mg, 1.50 mmol) was added, and the resulting mixture was stirred at room temperature for 16 h. To the reaction solution, 20 mL of saturated sodium bicarbonate aqueous solution was slowly added, and the mixture was extracted with DCM (40 mL × 3). The organic phase was washed with 50 mL of water, dried over anhydrous sodium sulfate and concentrated under reduced pressure. The crude product was separated and purified by silica gel column chromatography (dichloromethane/methanol (v/v) = 20 : 1) to obtain tert-butyl(3aR,6aS)-5-(4-(1-(1-((4-cyano-3-(trifluoromethyl)phenyl)amino)-2-methyl-1-oxopropan-2-yl)-1H-pyrazol-4-yl)piperidin-1-yl)hexahydrocyclopenta[c]pyrrole-2(1H)-carboxylate (1a) (0.25 g, yield: 81%).

$$\text{LCMS} \text{m} / \text{z} = 615.4 {\left[\text{M+1}\right]}^{+}$$

### Step 2: N-(4-cyano-3-(trifluoromethyl)phenyl)-2-methyl-2-(4-(1-((3aR,6aS)-octahydrocyclopenta[c]pyrrol-5-yl)piperidin-4-yl)-1H-pyrazol-1-yl)propanamide (1b)

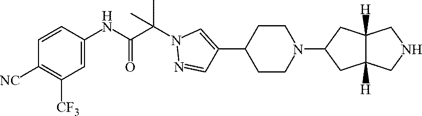

Tert-butyl(3aR,6aS)-5-(4-(1-(1-((4-cyano-3-(trifluoromethyl)phenyl)amino)-2-methyl-1-oxopropan-2-yl)- 1H-pyrazol-4-yl)piperidin-1-yl)hexahydrocyclopenta[c]pyrrole-2(1H)-carboxylate (1a) (0.25 g, 0.41 mmol) was dissolved in 10 mL of DCM. 6 mL of trifluoroacetic acid was added, and the mixture was stirred at room temperature for 3 h. After the reaction was completed, the reaction system was concentrated under reduced pressure, and the residue was dissolved in 30 mL of 4 mol/L aqueous NaOH. The reaction solution was extracted with DCM (40 mL × 3). The organic phase was washed with 50 mL of water, dried over anhydrous sodium sulfate and concentrated under reduced pressure to obtain crude N-(4-cyano-3-(trifluoromethyl)phenyl)-2-methyl-2-(4-(1-((3aR,6aS)-octahydrocyclopenta[c]pyrrol-5-yl)piperidin-4-yl)-1H-pyrazol-1-yl)propanamide (1b) (0.18 g).

$$\text{LCMS} \text{m} / \text{z} = 515.3 {\left[\text{M+1}\right]}^{+}$$

### Step 3: N-(4-cyano-3-(trifluoromethyl)phenyl)-2-(4-(1-((3aR,6aS)-2-(2-(2,6-dioxopiperidin-3-yl)-1,3-dioxoisoindolin-5-yl)octahydrocyclopenta[c]pyrrol-5-yl)piperidin-4-yl)-1H-pyrazol-1-yl)-2-methylpropanamide (Compound 1)

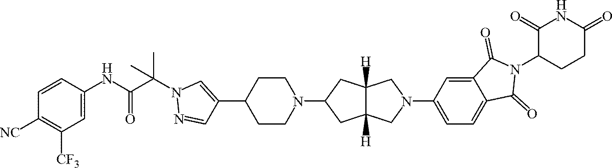

The crude N-(4-cyano-3-(trifluoromethyl)phenyl)-2-methyl-2-(4-(1-((3aR,6aS)-octahydrocyclopenta[c]pyrrol-5-yl)piperidin-4-yl)-1H-pyrazol-1-yl)propanamide (1b) (0.18 g) was dissolved in 10 mL of DMSO. 0.5 mL of DIPEA and 2-(2,6-dioxopiperidin-3-yl)-5-fluoroisoindoline-1,3-dione (see WO 2017197056 for the synthetic method) (145 mg, 0.53 mmol) were added, and the mixture was stirred at 80° C. for 5 h. The reaction solution was cooled to room temperature, and 20 mL of water was added. The mixture was filtered to collect a solid, which was washed with 10 mL of water, dissolved with 50 mL of dichloromethane, dried over anhydrous sodium sulfate and concentrated under reduced pressure, and then the crude product was separated and purified by silica gel column chromatography (dichloromethane/methanol (v/v) = 15 : 1) to obtain N-(4-cyano-3-(trifluoromethyl)phenyl)-2-(4-(1-((3aR,6aS)-2-(2-(2,6-dioxopiperidin-3-yl)-1,3-dioxoisoindolin-5-yl)octahydrocyclopenta[c]pyrrol-5-yl)piperidin-4-yl)- 1H-pyrazol- 1-yl)-2-methylpropanamide (compound 1) (140 mg, two-step yield calculated from compound 1a: 44%).

¹H NMR (400 MHz, CDC1₃) δ 9.78 (s, 1H), 8.57 (br.s, 1H), 7.99 - 7.90 (m, 1H), 7.80 - 7.68 (m, 2H), 7.64 (d, 1H), 7.60 (s, 1H), 7.45 (s, 1H), 7.00 - 6.95 (m, 1H), 6.69 (dd, 1H), 4.92 (dd, 1H), 3.66 - 3.52 (m, 2H), 3.44 - 3.32 (m, 2H), 3.19 - 3.03 (m, 2H), 2.92 - 2.63 (m, 6H), 2.59 - 2.45 (m, 1H), 2.35 - 2.21 (m, 2H), 2.18 - 2.01 (m, 3H), 1.97 - 1.85 (m, 8H), 1.84 - 1.62 (m, 2H), 1.60 - 1.47 (m, 2H).

$$\text{LCMS} \text{m} / \text{z} = 771.3 {\left[\text{M+1}\right]}^{+}$$

### Example 2

### N-cyano-3-(trifluoromethyl)phenyl)-2-(1-((1-(2-(2,6-dioxopiperidin-3-yl)-1,3-dioxoisoindolin-5-yl)pyrrolidin-3-yl)methyl)piperidin-4-yl)-1H-pyrazol-1-yl)-2-methylpropanamide (Compound 2)

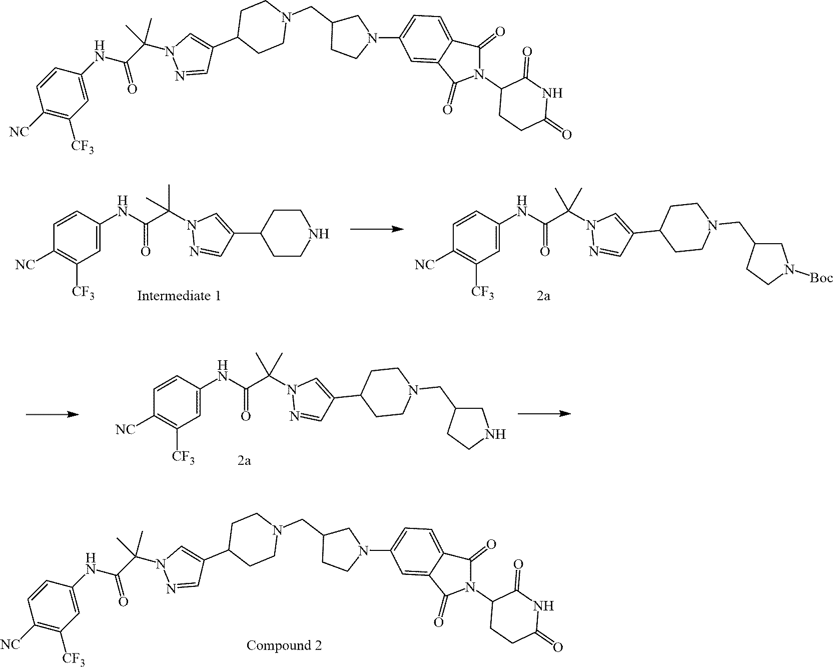

### Step 1: Tert-butyl 3-((4-(1-(1-((4-cyano-3-(trifluoromethyl)phenyl)amino)-2-methyl-1-oxopropan-2-yl)-1H-pyrazol-4-yl)piperidin-1-yl)methyl)pyrrolidine-1-carboxylate (2a)

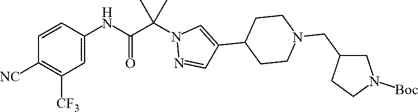

N-cyano-3-(trifluoromethyl)phenyl)-2-methyl-2-(piperidin-4-yl)-1H-pyrazol-1-yl)propanamide (intermediate 1) (202 mg, 0.50 mmol) was dissolved in 15 mL of DCE. Tert-butyl 3-formylpyrrole-1-carboxylate (200 mg, 1.00 mmol) was added, and the mixture was stirred at room temperature for 0.5 h. After that, sodium triacetoxyborohydride (318 mg, 1.5 mmol) was added, and the resulting mixture was stirred at room temperature for 16 h. To the reaction solution, 20 mL of saturated sodium bicarbonate aqueous solution was slowly added, and the mixture was extracted with DCM (40 mL × 3). The organic phase was washed with 50 mL of water, dried over anhydrous sodium sulfate and concentrated under reduced pressure. The crude product was separated and purified by silica gel column chromatography (dichloromethane/methanol (v/v) = 20 : 1) to obtain tert-butyl 3-((4-(1-(1-((4-cyano-3-(trifluoromethyl)phenyl)amino)-2-methyl-1-oxopropan-2-yl)-1H-pyrazol-4-yl)piperidin-1-yl)methyl)pyrrolidine-1-carboxylate (2a) (260 g, yield: 88%).

$$\text{LCMS} \text{m} / \text{z} = 589.2 {\left[\text{M+1}\right]}^{+}$$

### Step 2: N-(4-cyano-3-(trifluoromethyl)phenyl)-2-methyl-2-(4-(1-(pyrrolidin-3-ylmethyl)piperidin-4-yl)-1H-pyrazol-1-yl)propanamide (2b)

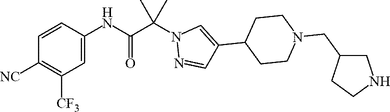

Tert-butyl 3-((4-(1-(1-((4-cyano-3-(trifluoromethyl)phenyl)amino)-2-methyl-1-oxopropan-2-yl)-1H-pyrazol-4-yl)piperidin-1-yl)methyl)pyrrolidine-1-carboxylate (2a) (0.26 g, 0.44 mmol) was dissolved in 10 mL of DCM. 6 mL of trifluoroacetic acid was added, and the mixture was stirred at room temperature for 3 h. After the reaction was completed, the reaction system was directly subjected to rotary evaporation, and the residue was dissolved in 30 mL of 4 mol/L aqueous NaOH. The reaction solution was extracted with DCM (40 mL × 3). The organic phase was washed with 50 mL of water, dried over anhydrous sodium sulfate and concentrated under reduced pressure to obtain crude N-(4-cyano-3-(trifluoromethyl)phenyl)-2-methyl-2-(4-(1-(pyrrolidin-3-ylmethyl)piperidin-4-yl)-1H-pyrazol-1-yl)propanamide (2b) (0.20 g).

$$\text{LCMS} \text{m} / \text{z} = 489.2 {\left[\text{M+1}\right]}^{+}$$

### Step 3: N-(4-cyano-3-(trifluoromethyl)phenyl)-2-(4-(1-((1-(2-(2,6-dioxopiperidin-3-yl)-1,3-dioxoisoindolin-5-yl)pyrrolidin-3-yl)methyl)piperidin-4-yl)-1H-pyrazol-1-yl)-2-methylpropanamide (Compound 2)

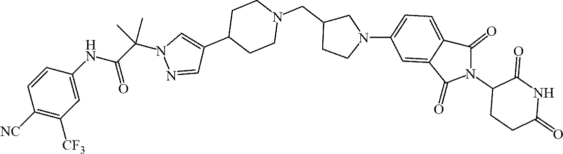

The crude N-(4-cyano-3-(trifluoromethyl)phenyl)-2-methyl-2-(4-(1-(pyrrolidin-3-ylmethyl)piperidin-4-yl)-1H-pyrazol-1-yl)propanamide (2b) (0.18 g) was dissolved in 10 mL of DMSO. 0.5 mL of DIPEA and 2-(2,6-dioxopiperidin-3-yl)-5-fluoroisoindoline-1,3-dione (see WO 2017197056 for the synthetic method) (153 mg, 0.55 mmol) were added, and the mixture was stirred at 80° C. for 5 h. The reaction solution was cooled to room temperature, and 20 mL of water was added. The mixture was filtered to collect a solid, which was washed with 10 mL of water, dissolved with 50 mL of dichloromethane, dried over anhydrous sodium sulfate and concentrated under reduced pressure, and then the crude product was separated and purified by silica gel column chromatography (dichloromethane/methanol (v/v) = 15 : 1) to obtain N-(4-cyano-3-(trifluoromethyl)phenyl)-2-(4-(1-((1-(2-(2,6-dioxopiperidin-3-yl)-1,3-dioxoisoindolin-5-yl)pyrrolidin-3-yl)methyl)piperidin-4-yl)-1H-pyrazol-1-yl)-2-methylpropanamide (compound 2) (150 mg, two-step yield calculated from compound 2a: 51%).

¹H NMR (400 MHz, CDC1₃) δ 9.79 (s, 1H), 8.22 (s, 1H), 7.99 - 7.93 (m, 1H), 7.81 - 7.69 (m, 2H), 7.68 - 7.60 (m, 2H), 7.48 (s, 1H), 6.95 (d, 1H), 6.68 (dd, 1H), 4.93 (dd, 1H), 3.65 - 3.35 (m, 3H), 3.30 - 3.15 (m, 1H), 3.12 - 2.59 (m, 6H), 2.59 - 2.32 (m, 3H), 2.30 - 2.05 (m, 3H), 1.98 - 1.80 (m, 9H), 1.76 - 1.52 (m, 3H).

$$\text{LCMS} \text{m} / \text{z} = 745.3 {\left[\text{M+1}\right]}^{+}$$

### Example 3

### N-cyano-3-(trifluoromethyl)phenyl)-2-(1-(2-(2-(2,6-dioxopiperidin-3-yl)-1,3-dioxoisoindolin-5-yl)-2-azaspiro[3.5]nonan-7-yl)piperidin-4-yl)-1H-pyrazol-1-yl)-2-methylpropanamide (Compound 3)

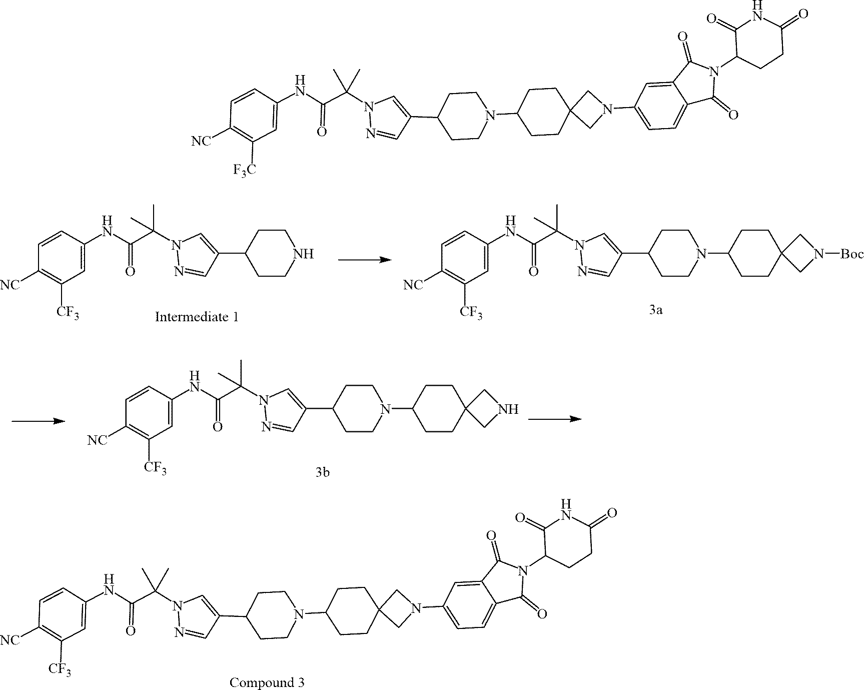

### Step 1: Tert-butyl 7-(4-(1-(1-((4-cyano-3-(trifluoromethyl)phenyl)amino)-2-methyl-1-oxopropan-2-yl)-1H-pyrazol-4-yl)piperidin-1-yl)-2-azaspiro[3.5]nonane-2-carboxylate (3a)

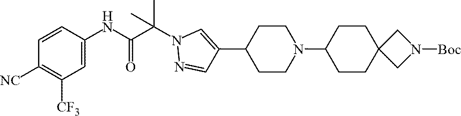

N-cyano-3-(trifluoromethyl)phenyl)-2-methyl-2-(piperidin-4-yl)-1H-pyrazol-1-yl)propanamide (intermediate 1) (202 mg, 0.50 mmol) was dissolved in 15 mL of DCE. 7-oxo-2-azaspiro[3.5]nonane-2-carboxylate (240 mg, 1.00 mmol) was added, and the mixture was stirred at room temperature for 0.5 h. After that, sodiumtriacetoxyborohydride (318 mg, 1.5 mmol) was added, and the resulting mixture was stirred at room temperature for 16 h. To the reaction solution, 20 mL of saturated sodium bicarbonate aqueous solution was slowly added, and the mixture was extracted with DCM (40 mL × 3). The organic phase was washed with 50 mL of water, dried over anhydrous sodium sulfate and concentrated under reduced pressure. The crude product was separated and purified by silica gel column chromatography (dichloromethane/methanol (v/v) = 20 : 1) to obtain tert-butyl 7-(4-(1-(1-((4-cyano-3-(trifluoromethyl)phenyl)amino)-2-methyl-1-oxopropan-2-yl)-1H-pyrazol-4-yl)piperidin-1-yl)-2-azaspiro[3.5]nonane-2-carboxylate (3a) (200 mg, yield: 64%).

$$\text{LCMS} \text{m} / \text{z} = 629.4 {\left[\text{M+1}\right]}^{+}$$

### Step 2: 2-(4-(1-(2-azaspiro[3.5]nonan-7-yl)piperidin-4-yl)-1H-pyrazol-1-yl)-N-(4-cyano-3-(trifluoromethyl)phenyl)-2-methylpropanamide (3b)

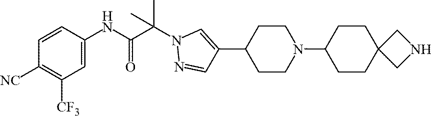

Tert-butyl 7-(4-(1-(1-((4-cyano-3-(trifluoromethyl)phenyl)amino)-2-methyl-1-oxopropan-2-yl)-1H-pyrazol-4-yl)piperidin-1-yl)-2-azaspiro[3.5]nonane-2-carboxylate (3a) (0.20 g, 0.32 mmol) was dissolved in 10 mL of DCM. 6 mL of trifluoroacetic acid was added, and the mixture was stirred at room temperature for 3 h. After the reaction was completed, the reaction system was directly concentrated under reduced pressure, and the residue was dissolved in 30 mL of 4 mol/L aqueous NaOH. The reaction solution was extracted with DCM (40 mL × 3). The organic phase was washed with 50 mL of water, dried over anhydrous sodium sulfate and concentrated under reduced pressure to obtain crude 2-(4-(1-(2-azaspiro[3.5]nonan-7-yl)piperidin-4-yl)-1H-pyrazol-1-yl)-N-(4-cyano-3-(trifluoromethyl)phenyl)-2-methylpropanamide (3b) (0.15 g).

$$\text{LCMS} \text{m} / \text{z} = 529.3 {\left[\text{M+1}\right]}^{+}$$

### Step 3: N-(4-cyano-3-(trifluoromethyl)phenyl)-2-(4-(1-(2-(2-(2,6-dioxopiperidin-3-yl)-1,3-dioxoisoindolin-5-yl)-2-azaspiro[3.5]nonan-7-yl)piperidin-4-yl)-1H-pyrazol-1-yl)-2-methylpropanamide (compound 3)

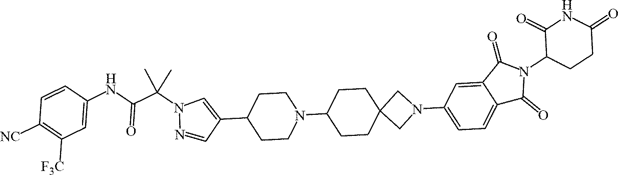

The crude 2-(4-(1-(2-azaspiro[3.5]nonan-7-yl)piperidin-4-yl)-1H-pyrazol-1-yl)-N-(4-cyano-3-(trifluoromethyl)phenyl)-2-methylpropanamide (3b) (0.15 g) was dissolved in 10 mL of DMSO. 0.5 mL of DIPEA and 2-(2,6-dioxopiperidin-3-yl)-5-fluoroisoindoline-1,3-dione (see WO 2017197056 for the synthetic method) (118 mg, 0.43 mmol) were added, and the mixture was stirred at 80° C. for 5 h. The reaction solution was cooled to room temperature, and 20 mL of water was added. The mixture was filtered to collect a solid, which was washed with 10 mL of water, dissolved with 50 mL of dichloromethane, dried over anhydrous sodium sulfate and concentrated under reduced pressure, and then the crude product was separated and purified by silica gel column chromatography (dichloromethane/methanol (v/v) = 15 : 1) to obtain N-(4-cyano-3-(trifluoromethyl)phenyl)-2-(4-(1-(2-(2-(2,6-dioxopiperidin-3-yl)-1,3-dioxoisoindolin-5-yl)-2-azaspiro[3.5]nonan-7-yl)piperidin-4-yl)-1H-pyrazol-1-yl)-2-methylpropanamide (compound 3) (120 mg, two-step yield calculated from compound 3a: 48%).

¹H NMR (400 MHz, CDC1₃) δ 9.78 (s, 1H), 8.22 (br.s, 1H), 7.97 - 7.93 (m, 1H), 7.81 - 7.69 (m, 2H), 7.66 - 7.59 (m, 2H), 7.46 (s, 1H), 6.76 (d, 1H), 6.49 (dd, 1H), 4.93 (dd, 1H), 3.73 (s, 2H), 3.68 (s, 2H), 3.16 - 2.95 (m, 2H), 2.93 - 2.67 (m, 3H), 2.59 -2.46 (m, 1H), 2.44 - 2.24 (m, 2H), 2.16 - 2.02 (m, 3H), 2.00 - 1.84 (m, 10H), 1.80 -1.52 (m, 5H), 1.45 - 1.31 (m, 2H).

$$\text{LCMS} \text{m} / \text{z} = 785.3 {\left[\text{M+1}\right]}^{+}$$

### Example 4

### N-cyano-3-(trifluoromethyl)phenyl]-1-[1-[[1-[2-(2,6-dioxo-3-piperidyl)-1,3-dioxo-isoindolin-5-yl]pyrrolidin-3-yl]methyl]-4-piperidyl]pyrazol-1-yl]cyclobutanecarboxamide (Compound 4)

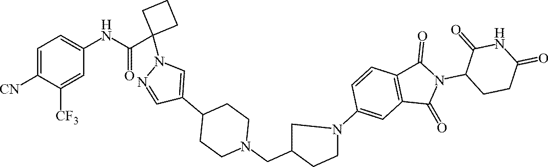 [SMILES: N#Cc1ccc(NC(=O)C2(n3cc(C4CCN(CC5CCN(c6ccc7c(c6)C(=O)N(C6CCC(=O)NC6=O)C7=O)C5)CC4)cn3)CCC2)cc1C(F)(F)F]

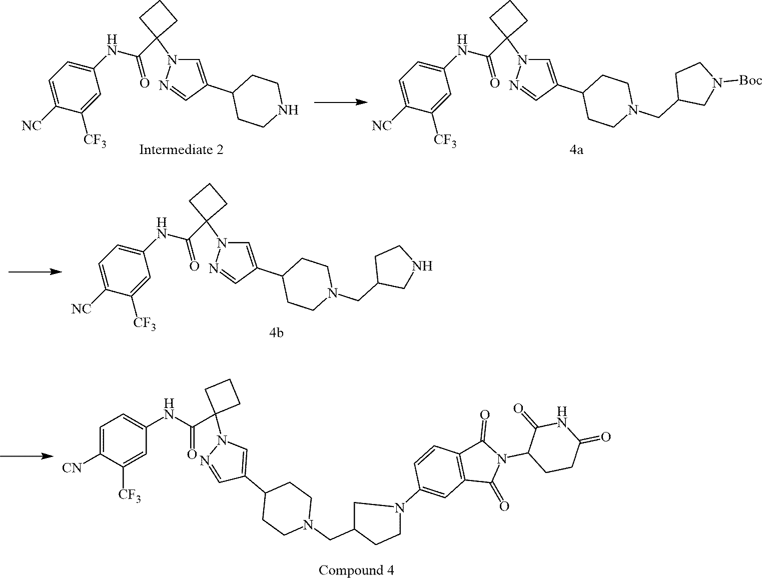

### Step 1: Tert-butyl 3-[[4-[1-[1-[[4-cyano-3-(trifluoromethyl)phenyl]carbamoyl]cyclobutyl]pyrazol-4-yl]-1-piperidyl]methyl]pyrrolidine-1-carboxylate (4a)

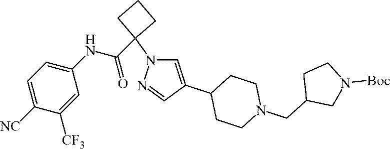

N-cyano-3-(trifluoromethyl)phenyl)-1-(piperidin-4-yl)-1H-pyrazol-1-yl)cyclobutane-1-carboxamide (intermediate 2) (0.100 g, 0.240 mmol) was dissolved in 3 mL of 1,2-dichloroethane. N-Boc-3-pyrrolecarboxaldehyde (0.0955 g, 0.479 mmol), glacial acetic acid (0.036 g, 0.599 mmol) and sodium triacetoxyborohydride (0.102 g, 0.481 mmol) were added successively. After the addition was completed, the mixture was reacted at room temperature for 16 h. The pH was adjusted to 9 by adding dropwise saturated sodium bicarbonate solution. The liquid separation was conducted, the aqueous layer was extracted with 20 mL of dichloromethane, and the organic layers were combined, dried over anhydrous sodium sulfate and concentrated under reduced pressure to obtain a crude product, which was separated and purified by silica gel column chromatography (DCM/MeOH (v/v) = 100/0-97/3) to obtain tert-butyl 3-[[4-[1-[1-[[4-cyano-3-(trifluoromethyl)phenyl]carbamoyl]cyclobutyl]pyrazol-4-yl]-1-piperidyl]methyl]pyrrolidine-1-carboxylate (4a) (0.08 g, yield: 56%).

$$\text{LCMS} \text{m} / \text{z} = 601.3 {\left[\text{M+1}\right]}^{+}$$

### Step 2: N-[4-cyano-3-(trifluoromethyl)phenyl]-1-[4-[1-(pyrrolidin-3-ylmethyl)-4-piperidyl]pyrazol-1-yl]cyclobutanecarboxamide (4b)

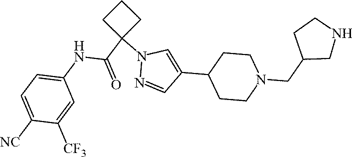

Tert-butyl 3-[[4-[1-[1-[[4-cyano-3-(trifluoromethyl)phenyl]carbamoyl]cyclobutyl]pyrazol-4-yl]-1-piperidyl]methyl]pyrrolidine-1-carboxylate (4a) (0.08 g, 0.133 mmol) was dissolved in 2 mL of dichloromethane. 1 mL of trifluoroacetic acid was added, and the mixture was reacted at room temperature for 1 h. The reaction solution was concentrated under reduced pressure. 20 mL of dichloromethane was added to the residue, and the pH was adjusted to 9 with saturated sodium bicarbonate. The liquid separation was conducted, the aqueous layer was extracted with 10 mL of dichloromethane, and the organic layers were combined, dried over anhydrous sodium sulfate and concentrated under reduced pressure to obtain crude N-[4-cyano-3-(trifluoromethyl)phenyl]-1-[4-[1-(pyrrolidin-3-ylmethyl)-4-piperidyl]pyrazol-1-yl]cyclobutanecarboxamide (4b) (0.0667 g).

$$\text{LCMS} \text{m} / \text{z} = 501.3 {\left[\text{M+1}\right]}^{+}$$

### Step 3: N-[4-cyano-3-(trifluoromethyl)phenyl]-1-[4-[1-[[1-[2-(2,6-dioxo-3-piperidyl)-1,3-dioxo-isoindolin-5-yl]pyrrolidin-3-yl]methyl]-4-piperidyl]pyrazol-1-yl]cyclobutanecarboxamide (Compound 4)

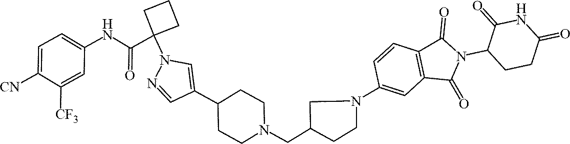 [SMILES: N#Cc1ccc(NC(=O)C2(n3cc(C4CCN(CC5CCN(c6ccc7c(c6)C(=O)N(C6CCC(=O)NC6=O)C7=O)C5)CC4)cn3)CCC2)cc1C(F)(F)F]

The crude N-[4-cyano-3-(trifluoromethyl)phenyl]-1-[4-[1-(pyrrolidin-3-ylmethyl)-4-piperidyl]pyrazol-1-yl]cyclobutanecarboxamide (4b) (0.060 g, 0.12 mmol) was dissolved in 3 mL of dimethylsulfoxide. 2-(2,6-dioxopiperidin-3-yl)-5-fluoroisoindoline-1,3-dione (see WO 2017197056 for the synthetic method) (0.036 g, 0.13 mmol) and diisopropylethylamine (0.031 g, 0.24 mmol) were added, and the mixture was stirred at 90° C. for 2 h. The reaction solution was cooled to room temperature. 5 mL of water was added, and the mixture was stirred for 2 min and filtered. The filter cake was washed with 10 mL of water, then collected and dissolved with dichloromethane (30 mL). The resultant was washed with saturated aqueoussodium chloride solution (10 mL), dried over anhydrous sodium sulfate and filtered. The filtrate was concentrated under reduced pressure, and the crude product was separated and purified by silica gel column chromatography (methanol/dichloromethane (v/v) = 0 : 1-1 : 19) to obtain N-[4-cyano-3-(trifluoromethyl)phenyl]-1-[4-[1-[[1-[2-(2,6-dioxo-3-piperidyl)-1,3-dioxo-isoindolin-5-yl]pyrrolidin-3-yl]methyl]-4-piperidyl]pyrazol-1-yl]cyclobutanecarboxamide (compound 4) (0.040 g, yield: 44%).

¹H NMR (400 MHz, CDC1₃) δ 9.01 (s, 1H), 8.17 (s, 1H), 7.98 - 7.92 (m, 1H), 7.79 - 7.68 (m, 2H), 7.68 - 7.57 (m, 2H), 7.41 (s, 1H), 6.95 (d, 1H), 6.72 - 6.65 (m, 1H), 4.94 (dd, 1H), 3.66 - 3.46 (m, 2H), 3.45 - 3.37 (m, 1H), 3.28 - 3.15 (m, 1H), 3.09 - 2.97 (m, 3H), 2.95 - 2.65 (m, 6H), 2.60 - 2.32 (m, 3H), 2.30 - 1.76 (m, 9H), 1.75 - 1.60 (m, 1H), 1.37 - 1.20 (m, 2H).

$$\text{LCMS} \text{m} / \text{z} = 757.3 {\left[\text{M+1}\right]}^{+}$$

### Example 5

### N-cyano-3-(trifluoromethyl)phenyl)-2-(1-(2-(2,6-dioxopiperidin-3-yl)-1,3-dioxoisoindolin-5-yl)piperidin-4-yl)-1H-pyrazol-1-yl)-2-methylpropanamide (Compound 5)

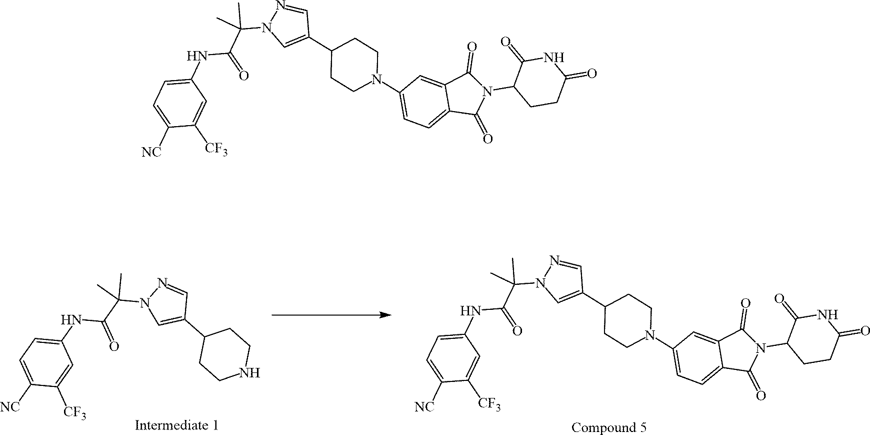

N-cyano-3-(trifluoromethyl)phenyl)-2-methyl-2-(piperidin-4-yl)-1H-pyrazol-1-yl)propanamide (intermediate 1) (0.1 g, 0.25 mmol) was dissolved in 5 mL of DMSO. 0.5 mL of DIPEA and 2-(2,6-dioxopiperidin-3-yl)-5-fluoroisoindoline-1,3-dione (see WO 2017197056 for the synthetic method) (90 mg, 0.33 mmol) was added, and the mixture was stirred at 85° C. for 3 h. The reaction solution was cooled to room temperature, and 30 mL of water was added. The mixture was filtered to collect a solid, which was washed with 10 mL of water, dissolved with 50 mL of dichloromethane, dried over anhydrous sodium sulfate and concentrated under reduced pressure, and then the crude product was separated and purified by silica gel column chromatography(dichloromethane/methanol (v/v) = 15 : 1) to obtain N-(4-cyano-3-(trifluoromethyl)phenyl)-2-(4-(1-(2-(2,6-dioxopiperidin-3-yl)-1,3-dioxoisoindolin-5-yl)piperidin-4-yl)-1H-pyrazol-1-yl)-2-methylpropanamide (compound 5) (90 mg, yield: 54%).

¹H NMR (400 MHz, CDC1₃) δ 9.82 (s, 1H), 8.12 (br.s, 1H), 7.96 - 7.93 (m, 1H), 7.82 - 7.66 (m, 3H), 7.63 (s, 1H), 7.48 (s, 1H), 7.31 (d, 1H), 7.08 (dd, 1H), 4.94 (dd, 1H), 4.08 - 3.98 (m, 2H), 3.16 - 3.04 (m, 2H), 2.94 - 2.67 (m, 4H), 2.19 - 2.01 (m, 3H), 1.92 (s, 6H), 1.78 - 1.65 (m, 2H).

$$\text{LCMS} \text{m} / \text{z} = 662.2 {\left[\text{M+1}\right]}^{+}$$

### Example 6

### N-cyano-3-(trifluoromethyl)phenyl]-1-[2-[1-[1-[2-(2,6-dioxo-3-piperidyl)-1,3-dioxo-isoindolin-5-yl]azetidin-3-yl]azetidin-3-yl]ethynyl]pyrazol-1-yl]cyclobutanecarboxamide (Compound 6)

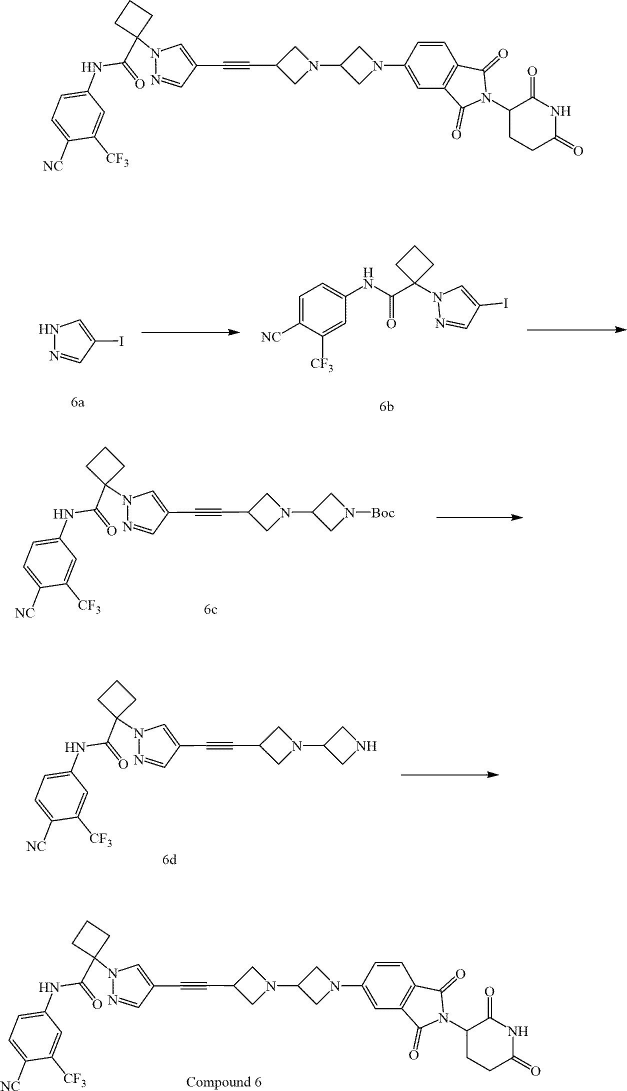

### Step 1: N-[4-cyano-3-(trifluoromethyl)phenyl]-1-(4-iodopyrazol-1-yl)cyclobutanecarboxamide (6b)

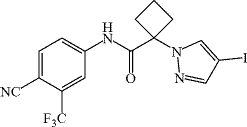

4-iodo-1H-pyrazole (6a) (0.559 g, 2.88 mmol) was dissolved in 10 mL of acetonitrile. 1-bromo-N-(4-cyano-3-(trifluoromethyl)phenyl)cyclobutane-1-carboxamide (see WO 2020063407 for the synthetic method) (1.00 g, 2.88 mmol) andcesium carbonate (1.03 g, 3.16 mmol) were added successively, and the mixture was reacted at 80° C. for 1 h under a microwave condition. The reaction solution was cooled to room temperature, and 20 mL of ethyl acetate and 10 mL of water were added. The liquid separation was conducted. The organic layer was dried over anhydrous sodium sulfate and concentrated under reduced pressure to obtain a crude product, which was separated and purified by silica gel column chromatography (petroleum ether/ethyl acetate (v/v) = 9 : 1) to obtain N-[4-cyano-3-(trifluoromethyl)phenyl]-1-(4-iodopyrazol-1-yl)cyclobutanecarboxamide (6b) (0.130 g, yield: 10%).

### Step 2: Tert-butyl 3-[3-[2-[1-[1-[[4-cyano-3-(trifluoromethyl)phenyl]carbamoyl]cyclobutyl]pyrazol-4-yl]ethynyl]azetidin-1-yl]azetidine-1-carboxylate (6c)

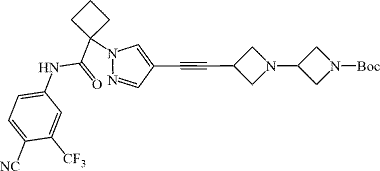

N-cyano-3-(trifluoromethyl)phenyl]-1-(4-iodopyrazol-1-yl)cyclobutanecarboxamide (6b) (0.100 g, 0.217 mmol) was dissolved in 3 mL of dichloromethane. Triethylamine (0.0660 g, 0.652 mmol) was added, PdCl₂(PPh₃)₂ (0.0150 g, 0.0213 mmol) and cuprous iodide (0.0062 g, 0.0326 mmol) were added successively under nitrogen atmosphere, and then a solution of tert-butyl 3-ethynyl-[1,3′-biazetidine]-1′-carboxylate (intermediate B) (0.0770 g, 0.326 mmol) in dichloromethane (1 mL) was slowly added dropwise. After the addition was completed, the reaction solution was reacted at room temperature for 16 h. 5 mL of water and 10 mL of dichloromethane were added. The liquid separation was conducted, and the organic layer was washed with 5 mL of saturated sodium chloride solution, dried over anhydrous sodium sulfate and concentrated under reduced pressure to obtain a crude product, which was separated and purified by silica gel column chromatography (ethyl acetate) to obtain tert-butyl 3-[3-[2-[1-[1-[[4-cyano-3-(trifluoromethyl)phenyl]carbamoyl]cyclobutyl]pyrazol-4-yl]ethynyl]azetidin-1-yl]azetidine-1-carboxylate (6c) (0.0350 g, yield: 28%).

### Step 3: 1-[4-[2-[1-(azetidin-3-yl)azetidin-3-yl]ethynyl]pyrazol-1-yl]-N-[4-cyano-3-(trifluoromethyl)phenyl]cyclobutanecarboxamide (6d)

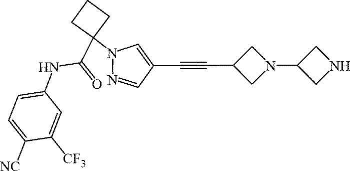

Tert-butyl 3-[3-[2-[1-[1-[[4-cyano-3-(trifluoromethyl)phenyl]carbamoyl]cyclobutyl]pyrazol-4-yl]ethynyl]azetidin-1-yl]azetidine-1-carboxylate (6c) (0.030 g, 0.053 mmol) was dissolved in 5 mL of dichloromethane. 2 mL of trifluoroacetic was added, and the mixture was reacted at room temperature for 1 h. The reaction solution was concentrated under reduced pressure. 20 mL of dichloromethane was added to the residue, and the pH was adjusted to 9 with saturated sodium bicarbonate. The liquid separation was conducted, the aqueous layer was extracted with 10 mL of dichloromethane, and the organic layers were combined, dried over anhydrous sodium sulfate and concentrated under reduced pressure to obtain crude 1-[4-[2-[1-(azetidin-3-yl)azetidin-3-yl]ethynyl]pyrazol-1-yl]-N-[4-cyano-3-(trifluoromethyl)phenyl]cyclobutanecarboxamide (6d) (0.020 g).

$$\text{LCMS} \text{m} / \text{z} = 469.2 {\left[\text{M+1}\right]}^{+}$$

### Step 4: N-[4-cyano-3-(trifluoromethyl)phenyl]-1-[4-[2-[1-[1-[2-(2,6-dioxo-3-piperidyl)-1,3-dioxo-isoindolin-5-yl]azetidin-3-yl]azetidin-3-yl]ethynyl]pyrazol-1-yl]cyclobutanecarboxamide (Compound 6)

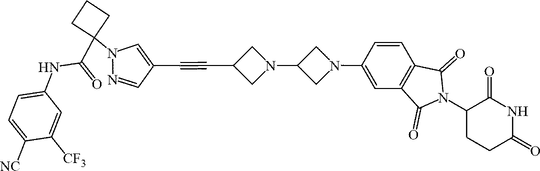

The crude 1-[4-[2-[1-(azetidin-3-yl)azetidin-3-yl]ethynyl]pyrazol-1-yl]-N-[4-cyano-3-(trifluoromethyl)phenyl]cyclobutanecarboxamide (6d) (0.020 g) was dissolved in dimethyl sulfoxide (3 mL). 2-(2,6-dioxopiperidin-3-yl)-5-fluoroisoindoline-1,3-dione (see WO 2017197056 for the synthetic method) (0.013 g, 0.047 mmol) and diisopropylethylamine (0.011 g, 0.085 mmol) were added, and the mixture was stirred at 90° C. for 2 h. The reaction solution was cooled to room temperature. 5 mL of water was added, and the mixture was stirred for 2 min and filtered. The filter cake was washed with 10 mL of water, then collected and dissolved with dichloromethane (30 mL). The resultant was washed with 10 mL of saturatedaqueous sodium chloride solution, dried over anhydrous sodium sulfate and filtered. The filtrate was concentrated under reduced pressure, and the crude product was separated and purified by silica gel column chromatography (methanol/dichloromethane (v/v) = 0 : 1-1 : 19) to obtain N-[4-cyano-3-(trifluoromethyl)phenyl]-1-[4-[2-[1-[1-[2-(2,6-dioxo-3-piperidyl)-1,3-dioxo-isoindolin-5-yl]azetidin-3-yl]azetidin-3-yl]ethynyl]pyrazol-1-yl]cyclobutanecarboxamide (compound 6) (0.020 g, two-step yield calculated from compound 6c: 52%).

¹H NMR (400 MHz, CDC1₃) δ 8.11 - 7.97 (m, 2H), 7.95 - 7.87 (m, 1H), 7.80 -7.74 (m, 1H), 7.72 - 7.64 (m, 2H), 7.58 - 7.50 (m, 1H), 6.83 - 6.77 (m, 1H), 6.58 - 6.51 (m, 1H), 4.93 (dd, 1H), 4.20 - 3.26 (m, 10H), 2.94 - 2.67 (m, 4H), 2.56 - 2.45 (m, 1H), 2.44 - 2.28 (m, 2H), 2.28 - 2.17 (m, 1H), 2.17 - 2.08 (m, 1H), 2.06 - 1.96 (m, 1H).

$$\text{LCMS} \text{m} / \text{z} = 725.2 {\left[\text{M+1}\right]}^{+}$$

### Example 7

### N-cyano-3-(trifluoromethyl)phenyl]-1-[2-[1-[2-(2,6-dioxo-3-piperidyl)-1,3-dioxo-isoindolin-5-yl]azetidin-3-yl]ethynyl]pyrazol-1-yl]cyclobutanecarboxamide (Compound 7)

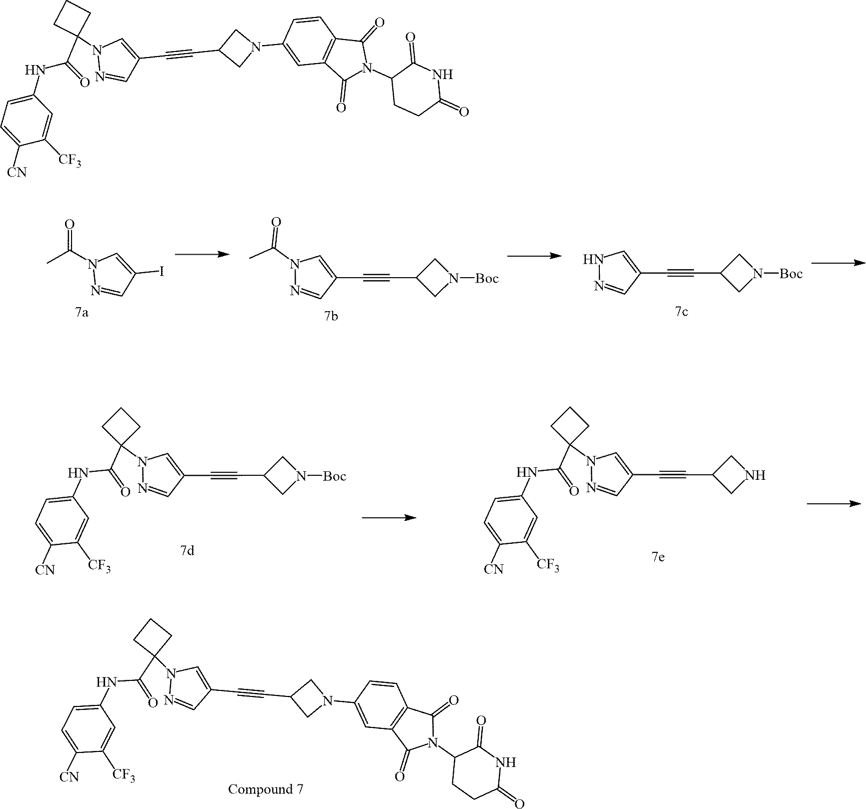

### Step 1: Tert-butyl 3-[2-(1-acetylpyrazol-4-yl)ethynyl]azetidine-1-carboxylate (7b)

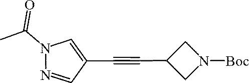 [SMILES: CC(=O)n1cc(C#CC2CN(C(=O)OC(C)(C)C)C2)cn1]

1-acetyl-4-iodo-1H-pyrazole (7a) (0.200 g, 0.847 mmol) was dissolved in 3 mL of dichloromethane. Triethylamine (0.257 g, 2.54 mmol) was added, PdCl₂(PPh₃)₂ (0.06 g, 0.0855 mmol) and cuprous iodide (0.0242 g, 0.127 mmol) were added successively under nitrogen atmosphere, and then a solution of tert-butyl 3-ethynyl-1-azetidinecarboxylate (0.200 g, 1.10 mmol) in dichloromethane (1 mL) was slowly added dropwise. After the addition was completed, the mixture was reacted at room temperature for 16 h. 5 mL of water and 10 mL of dichloromethane were added to the reaction system. The liquid separation was conducted, and the organic layer was washed with 5 mL of saturated sodium chloride solution, dried over anhydrous sodium sulfate and concentrated under reduced pressure to obtain a crude product, which was separated and purified by silica gel column chromatography (petroleum ether/ethyl acetate (v/v) = 5/1) to obtain tert-butyl 3-[2-(1-acetylpyrazol-4-yl)ethynyl]azetidine-1-carboxylate (7b) (0.190 g, yield: 78%).

¹H NMR (400 MHz, CDC1₃) δ 8.27 (s, 1H), 7.69 (s, 1H), 4.23 - 4.16 (m, 2H), 4.04 - 3.96 (m, 2H), 3.56 - 3.45 (m, 1H), 2.69 (s, 3H), 1.45 (s, 9H).

### Step 2: Tert-butyl 3-[2-(1H-pyrazol-4-yl)ethynyl]azetidine-1-carboxylate (7c)

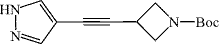 [SMILES: CC(C)(C)OC(=O)N1CC(C#Cc2cn[nH]c2)C1]

Tert-butyl 3-[2-(1-acetylpyrazol-4-yl)ethynyl]azetidine-1-carboxylate (7b) (0.190 g, 0.657 mmol) was dissolved in 5 mL of anhydrous methanol. Potassium carbonate (0.182 g, 1.32 mmol) was added, and The mixture was stirred at room temperature for 5 min. 10 mL of water and 20 mL of ethyl acetate were added to the reaction solution. The liquid separation was conducted, and the organic layer was washed with 10 mL of saturated sodium chloride solution, dried over anhydrous sodium sulfate and concentrated under reduced pressure to obtain crude tert-butyl 3-[2-(1H-pyrazol-4-yl)ethynyl]azetidine-1-carboxylate (7c) (0.150 g).

### Step 3: Tert-butyl 3-[2-[1-[1-[[4-cyano-3-(trifluoromethyl)phenyl]carbamoyl]cyclobutyl]pyrazol-4-yl]ethynyl]azetidine-1-carboxylate (7d)

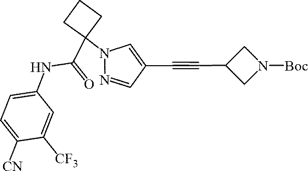 [SMILES: CC(C)(C)OC(=O)N1CC(C#Cc2cnn(C3(C(=O)Nc4ccc(C#N)c(C(F)(F)F)c4)CCC3)c2)C1]

The crude tert-butyl 3-[2-(1H-pyrazol-4-yl)ethynyl]azetidine-1-carboxylate (7c) (0.142 g) was dissolved in 10 mL of acetonitrile. 1-bromo-N-(4-cyano-3-(trifluoromethyl)phenyl)cyclobutane-1-carboxamide (see WO 2020063407 for the synthetic method) (0.200 g, 0.576 mmol) and cesium carbonate (0.207 g, 0.635 mmol) were added, and the mixture was reacted at 80° C. for 1 h under a microwave condition. The reaction solution was cooled to room temperature. 5 mL of water and 10 mL of dichloromethane were added. The liquid separation was conducted, and the organic layer was washed with 10 mL of saturated sodium chloride solution, dried over anhydrous sodium sulfate and concentrated under reduced pressure. The crude product was separated and purified by silica gel column chromatography (petroleum ether/ethyl acetate (v/v) = 9/1) to obtain tert-butyl 3-[2-[1-[1-[[4-cyano-3-(trifluoromethyl)phenyl]carbamoyl]cyclobutyl]pyrazol-4-yl]ethynyl]azetidine-1-carboxylate (7d) (0.130 g, two-step yield calculated from compound 7b: 41%).

### Step 4: 1-[4- [2-(azetidin-3 -yl)ethynyl]pyrazol-1 -yl] -N- [4-cyano-3 -(trifluoromethyl)phenyl]cyclobutanecarboxamide (7e)

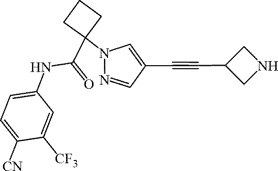 [SMILES: N#Cc1ccc(NC(=O)C2(n3cc(C#CC4CNC4)cn3)CCC2)cc1C(F)(F)F]

Tert-butyl 3-[2-[1-[1-[[4-cyano-3-(trifluoromethyl)phenyl]carbamoyl]cyclobutyl]pyrazol-4-yl]ethynyl]azetidine-1-carboxylate (7d) (0.050 g, 0.097 mmol) was dissolved in 5 mL of dichloromethane. 2 mL of trifluoroacetic was added, and the mixture was reacted at room temperature for 1 h. The reaction solution was concentrated under reduced pressure. 20 mL of dichloromethane was added to the residue, and the pH was adjusted to 10 with saturated sodium bicarbonate. The liquid separation was conducted, the aqueous layer was extracted with 10 mL of dichloromethane, and the organic layers were combined, dried over anhydrous sodium sulfate and concentrated under reduced pressure to obtaincrude 1-[4-[2-(azetidin-3-yl)ethynyl]pyrazol-1-yl]-N-[4-cyano-3-(trifluoromethyl)phenyl]cyclobutanecarboxamide (7e) (0.040 g).

$$\text{LCMS} \text{m} / \text{z} = 414.1 {\left[\text{M+1}\right]}^{+}$$

### Step 5: N-[4-cyano-3-(trifluoromethyl)phenyl]-1-[4-[2-[1-[2-(2,6-dioxo-3-piperidyl)-1,3-dioxo-isoindolin-5-yl]azetidin-3-yl]ethynyl]pyrazol-1-yl]cyclobutanecarboxamide (Compound 7)

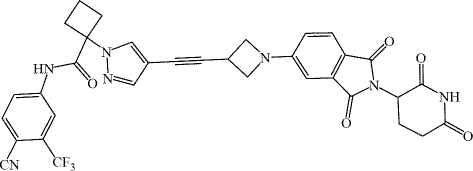 [SMILES: N#Cc1ccc(NC(=O)C2(n3cc(C#CC4CN(c5ccc6c(c5)C(=O)N(C5CCC(=O)NC5=O)C6=O)C4)cn3)CCC2)cc1C(F)(F)F]

The crude 1-[4-[2-(azetidin-3-yl)ethynyl]pyrazol-1-yl]-N-[4-cyano-3-(trifluoromethyl)phenyl]cyclobutanecarboxamide (7e) (0.030 g) was dissolved in dimethyl sulfoxide (3 mL). 2-(2,6-dioxopiperidin-3-yl)-5-fluoroisoindoline-1,3-dione (see WO 2017197056 for the synthetic method) (0.022 g, 0.080 mmol) and diisopropylethylamine (0.019 g, 0.15 mmol) were added, and the mixture was stirred at 90° C. for 2 h. The reaction solution was cooled to room temperature. 5 mL of water was added, and the mixture was stirred for 2 min and filtered. The filter cake was washed with 10 mL of water, then collected and dissolved with dichloromethane (30 mL). The organic phase was washed with 10 mL of saturated sodium chloride solution, dried over anhydrous sodium sulfate and filtered. The filtrate was concentrated under reduced pressure, and the crude product was separated and purified by silica gel column chromatography (petroleum ether/ethyl acetate (v/v) = 3/7) to obtain N-[4-cyano-3-(trifluoromethyl)phenyl]-1-[4-[2-[1-[2-(2,6-dioxo-3-piperidyl)-1,3-dioxo-isoindolin-5-yl]azetidin-3-yl]ethynyl]pyrazol-1-yl]cyclobutanecarboxamide (compound 7) (0.010 g, two-step yield calculated from compound 7d: 21%).

¹H NMR (400 MHz, CDC1₃) δ 8.83 (s, 1H), 8.23 (s, 1H), 7.97 - 7.91 (m, 1H), 7.83 - 7.61 (m, 5H), 6.80 (d, 1H), 6.55 (dd, 1H), 4.94 (dd, 1H), 4.40 - 4.31 (m, 2H), 4.11 -4.00 (m, 2H), 3.87 - 3.74 (m, 1H), 3.11 - 2.99 (m, 2H), 2.96 - 2.67 (m, 5H), 2.26 - 1.96 (m, 3H).

$$\text{LCMS} \text{m} / \text{z} = 670.3 {\left[\text{M+1}\right]}^{+}$$

### Example 8

### N-cyano-3-(trifluoromethyl)phenyl)-2-((1′-(2-(2,6-dioxopiperidin-3-yl)-1,3-dioxoisoindolin-5-yl)-[1,3′-biazetidin]-3-yl)ethynyl)-1H-pyrazol-1-yl)-2-methylpropanamide (Compound 8)

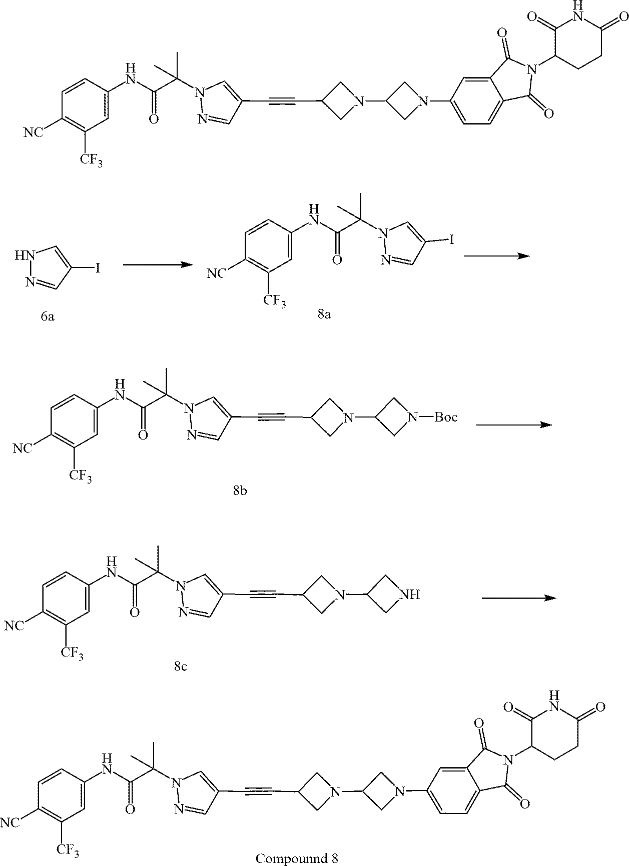

### Step 1: N-(4-cyano-3-(trifluoromethyl)phenyl)-2-(4-iodo-1H-pyrazol-1-yl)-2-methylpropanamide (8a)

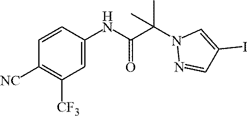

2-bromo-N-(4-cyano-3-(trifluoromethyl)phenyl)-2-methylpropanamide (see WO 2020063407 for the synthetic method) (1.72 g, 5.13 mmol) was dissolved in 35 mL of acetonitrile. 4-iodo-1H-pyrazole (6a) (1.0 g, 5.15 mmol) and cesium carbonate (3.36 g, 10.30 mmol) were added, and the mixture was stirred at 50° C. for 3 h. The reaction solution was cooled to room temperature. 50 mL of water was added, and the mixture was extracted with DCM (40 mL × 3). The organic phase was washed with 50 mL of water, dried over anhydrous sodium sulfate and concentrated under reduced pressure. The crude product was separated and purified by silica gel column chromatography (petroleum ether/ethyl acetate (v/v) = 4 : 1) to obtain N-(4-cyano-3-(trifluoromethyl)phenyl)-2-(4-iodo-1H-pyrazol-1-yl)-2-methylpropanamide (8a) (1.5 g, yield: 65%).

¹H NMR (400 MHz, DMSO-d₆) δ 10.06 (s, 1H), 8.26 (d, 1H), 8.18 (s, 1H), 8.15 (dd, 1H), 8.08 (d, 1H), 7.62 (s, 1H), 1.81 (s, 6H).

$$\text{LCMS} \text{m} / \text{z} = 449.0 {\left[\text{M+1}\right]}^{+}$$

### Step 2: Tert-butyl 3-((1-(1-((4-cyano-3-(trifluoromethyl)phenyl)amino)-2-methyl-1-oxopropan-2-yl)-1H-pyrazol-4-yl)ethynyl)-[1,3′-biazetidine]-1′-carboxylate (8b)

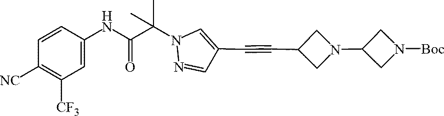

N-cyano-3-(trifluoromethyl)phenyl)-2-iodo-1H-pyrazol-1-yl)-2-methylpropanamide (8a) (224 mg, 0.50 mmol) was dissolved in 10 mL of dichloromethane. Triethylamine (150 mg, 1.48 mmol) was added, PdCl₂(PPh₃)₂ (35 mg, 0.05 mmol) and cuprous iodide (10 mg, 0.05 mmol) were added successively under nitrogen atmosphere, and then a solution of tert-butyl 3-ethynyl-[1,3′-biazetidine]-1′-carboxylate (intermediate B) (178 mg, 0.75 mmol) in dichloromethane (2 mL) was slowly added dropwise. After the addition was completed, the reaction solution was reacted at room temperature for 16 h. 15 mL of water and 10 mL of dichloromethane were added to the reaction solution. The liquid separation was conducted, and the organic layer was washed with 5 mL of saturated sodium chloride solution, dried over anhydrous sodium sulfate and concentrated under reduced pressure. The resulting crude product was separated and purified by silica gel column chromatography (petroleum ether/ethyl acetate (v/v) = 4 : 1) to obtain tert-butyl 3-((1-(1-((4-cyano-3-(trifluoromethyl)phenyl)amino)-2-methyl-1-oxopropan-2-yl)-1H-pyrazol-4-yl)ethynyl)-[1,3′-biazetidine]-1′-carboxylate (8b) (260 mg, yield: 93%).

$$\text{LCMS} \text{m} / \text{z} = 557.3 {\left[\text{M+1}\right]}^{+}$$

### Step 3: 2-(4-([1,3′-biazetidin]-3-ylethynyl)-1H-pyrazol-1-yl)-N-(4-cyano-3-(trifluoromethyl)phenyl)-2-methylpropanamide (8c)

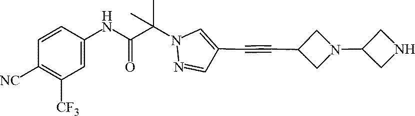

Tert-butyl 3-((1-(1-((4-cyano-3-(trifluoromethyl)phenyl)amino)-2-methyl-1-oxopropan-2-yl)-1 H-pyrazol-4-yl)ethynyl)- [1,3 ‘-biazetidine] -1′-carboxylate (8b) (0.24 g, 0.43 mmol) was dissolved in 10 mL of DCM. 6 mL of trifluoroacetic acid was added, and the mixture was stirred at room temperature for 3 h. After the reaction was completed, the reaction system was directly concentrated under reduced pressure, and the residue was dissolved in 20 mL of 4 mol/L aqueous NaOH. The reaction solutionwas extracted with DCM (40 ml × 3). The organic phase was washed with 50 mL of water, dried over anhydrous sodium sulfate and concentrated under reduced pressure to obtain crude 2-(4-([1,3′-biazetidin]-3-ylethynyl)-1H-pyrazol-1-yl)-N-(4-cyano-3-(trifluoromethyl)phenyl)-2-methylpropanamide (8c) (0.18 g).

$$\text{LCMS} \text{m} / \text{z} = 457.1 {\left[\text{M+1}\right]}^{+}$$

### Step 4: N-(4-cyano-3-(trifluoromethyl)phenyl)-2-(4-((1′-(2-(2,6-dioxopiperidin-3-yl)-1,3-dioxoisoindolin-5-yl)-[1,3′-biazetidin]-3-yl)ethynyl)-1H-pyrazol-1-yl)-2-methylpropanamide (Compound 8)

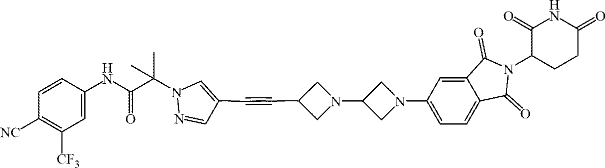

The crude 2-(4-([1,3′-biazetidin]-3-ylethynyl)-1H-pyrazol-1-yl)-N-(4-cyano-3-(trifluoromethyl)phenyl)-2-methylpropanamide (8c) (0.18 g) was dissolved in 10 mL of DMSO. 0.5 mL of DIPEA and 2-(2,6-dioxopiperidin-3-yl)-5-fluoroisoindoline-1,3-dione (see WO 2017197056 for the synthetic method) (181 mg, 0.66 mmol) were added, and the mixture was stirred at 80° C. for 5 h. The reaction solution was cooled to room temperature, and 20 mL of water was added. The mixture was filtered to collect a solid, which was washed with 20 mL of water, dissolved with 50 mL of dichloromethane, dried over anhydrous sodium sulfate and concentrated under reduced pressure, and then the crude product was separated and purified by silica gel column chromatography (dichloromethane/methanol (v/v) = 15 : 1) to obtain N-(4-cyano-3-(trifluoromethyl)phenyl)-2-(4-((1′-(2-(2,6-dioxopiperidin-3-yl)-1,3-dioxoisoindolin-5-yl)-[1,3′-biazetidin]-3-yl)ethynyl)-1H-pyrazol-1-yl)-2-methylpropanamide (compound 8) (18 mg, two-step yield calculated from compound 8b: 6%).

¹H NMR (400 MHz, CDC1₃) δ 9.47 (s, 1H), 8.31 (s, 1H), 7.96 - 7.90 (m, 1H), 7.84 - 7.70 (m, 4H), 7.64 (d, 1H), 6.78 (d, 1H), 6.52 (dd, 1H), 4.92 (dd, 1H), 4.11 - 4.01 (m, 2H), 3.96 - 3.84 (m, 2H), 3.83 - 3.63 (m, 3H), 3.58 - 3.47 (m, 1H), 3.39 - 3.25 (m, 2H), 2.94 - 2.64 (m, 3H), 2.17 - 2.07 (m, 1H), 1.92 (s, 6H).

$$\text{LCMS} \text{m} / \text{z} = 713.3 {\left[\text{M+1}\right]}^{+}$$

### Example 9

### N-cyano-3-(trifluoromethyl)phenyl)-2-((1-(2-(2,6-dioxopiperidin-3-yl)-1,3-dioxoisoindolin-5-yl)azetidin-3-yl)ethynyl)-1H-pyrazol-1-yl)-2-methylpropanamide (Compound 9)

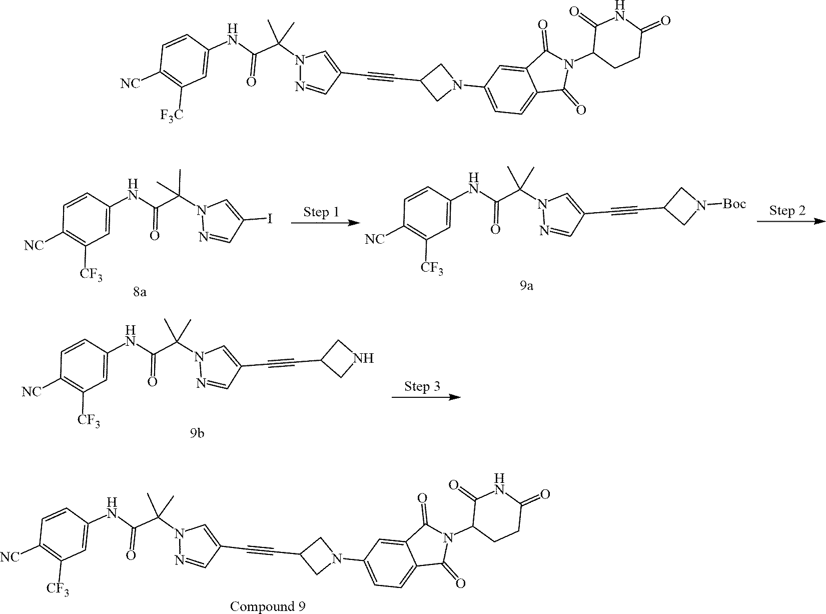

### Step 1: Tert-butyl 3-((1-(1-((4-cyano-3-(trifluoromethyl)phenyl)amino)-2-methyl-1-oxopropan-2-yl)-1H-pyrazol-4-yl)ethynyl)azetidine-1-carboxylate (9a)

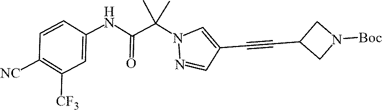

N-cyano-3-(trifluoromethyl)phenyl)-2-iodo-1H-pyrazol-1-yl)-2-methylpropanamide (8a) (224 mg, 0.50 mmol) was dissolved in 10 mL of dichloromethane. Triethylamine (150 mg, 1.48 mmol) was added, PdCl₂(PPh₃)₂ (35 mg, 0.05 mmol) and cuprous iodide (10 mg, 0.05 mmol) were added successively under nitrogen atmosphere, and then a solution of tert-butyl 3-ethynylazetidine-1-carboxylate (136 mg, 0.75 mmol) in dichloromethane (2 mL) was slowly added dropwise. After the addition was completed, the reaction solution was reacted at room temperature for 16 h. 15 mL of water and 10 mL of dichloromethane were added to the reaction solution. The liquid separation was conducted, and the organic layer was washed with 5 mL of saturated sodium chloride solution, dried over anhydrous sodium sulfate and concentrated under reduced pressure. The residue was separated and purified by silica gel column chromatography (petroleum ether/ethyl acetate (v/v) = 4 : 1) to obtain tert-butyl 3-((1-(1-((4-cyano-3-(trifluoromethyl)phenyl)amino)-2-methyl-1-oxopropan-2-yl)-1H-pyrazol-4-yl)ethynyl)azetidine-1-carboxylate (9a) (230 mg, yield: 92%).

### Step 2: 2-(4-(azetidin-3-ylethynyl)-1H-pyrazol-1-yl)-N-(4-cyano-3-(trifluoromethyl)phenyl)-2-methylpropanamide (9b)

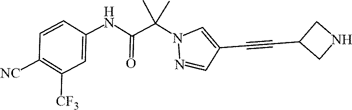

Tert-butyl 3-((1-(1-((4-cyano-3-(trifluoromethyl)phenyl)amino)-2-methyl-1-oxopropan-2-yl)-1H-pyrazol-4-yl)ethynyl)azetidine-1-carboxylate (9a) (0.23 g, 0.46 mmol) was dissolved in 10 mL of DCM. 6 mL of trifluoroacetic acid was added, and the mixture was stirred at room temperature for 3 h. After the reaction was completed, the system was directly concentrated under reduced pressure, and the residue was dissolved in 20 mL of 4 mol/L aqueous NaOH. The reaction solution was extracted with DCM (40 mL × 3). The organic phase was washed with 50 mL of water, dried over anhydrous sodium sulfate and concentrated under reduced pressure to obtain crude 2-(4-(azetidin-3-ylethynyl)-1H-pyrazol-1-yl)-N-(4-cyano-3-(trifluoromethyl)phenyl)-2-methylpropanamide (9b) (0.18 g).

$$\text{LCMS} \text{m} / \text{z} = 402.1 {\left[\text{M+1}\right]}^{+}$$

### Step 3: N-(4-cyano-3-(trifluoromethyl)phenyl)-2-(4-((1-(2-(2,6-dioxopiperidin-3-yl)-1,3-dioxoisoindofin-5-yl)azetidin-3-yl)ethynyl)-1H-pyrazol-1-yl)-2-methylpropanamide (Compound 9)

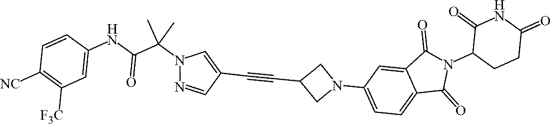

The crude 2-(4-(azetidin-3-ylethynyl)-1H-pyrazol-1-yl)-N-(4-cyano-3-(trifluoromethyl)phenyl)-2-methylpropanamide (9b) (0.17 g) was dissolved in 10 mL of DMSO. 0.5 mL of DIPEA and 2-(2,6-dioxopiperidin-3-yl)-5-fluoroisoindoline-1,3-dione (see WO 2017197056 for the synthetic method) (180 mg, 0.65 mmol) were added, and the mixture was stirred at 80° C. for 5 h. The reaction solution was cooled to room temperature, and 20 mL of water was added. The mixture was filtered to collect a solid, which was washed with 20 mL of water, dissolved with 50 mL of dichloromethane, dried over anhydrous sodium sulfate and concentrated under reduced pressure, and then the crude product was separated and purified by silica gel column chromatography (dichloromethane/methanol (v/v) = 15 : 1) to obtain N-(4-cyano-3-(trifluoromethyl)phenyl)-2-(4-((1-(2-(2,6-dioxopiperidin-3-yl)-1,3-dioxoisoindolin-5-yl)azetidin-3-yl)ethynyl)-1H-pyrazol-1-yl)-2-methylpropanamide (compound 9) (80 mg, two-step yield calculated from compound 9a: 28%).

¹H NMR (400 MHz, CDC1₃) δ 9.49 (s, 1H), 8.04 (s, 1H), 7.95 - 7.90 (m, 1H), 7.84 - 7.70 (m, 4H), 7.67 (d, 1H), 6.80 (d, 1H), 6.56 (dd, 1H), 4.94 (dd, 1H), 4.40 - 4.32 (m, 2H), 4.10 - 4.02 (m, 2H), 3.86 - 3.75 (m, 1H), 2.94 - 2.65 (m, 3H), 2.18 - 2.07 (m, 1H), 1.92 (s, 6H).

$$\text{LCMS} \text{m} / \text{z} = 658.2 {\left[\text{M+1}\right]}^{+}$$

### Example 10

### N-cyano-3-(trifluoromethyl)phenyl)-2-((1-(2-(2,6-dioxopiperidin-3-yl)-1,3-dioxoisoindolin-5-yl)piperidin-4-yl)ethynyl)-1H-pyrazol-1-yl)-2-methylpropanamide (Compound 10)

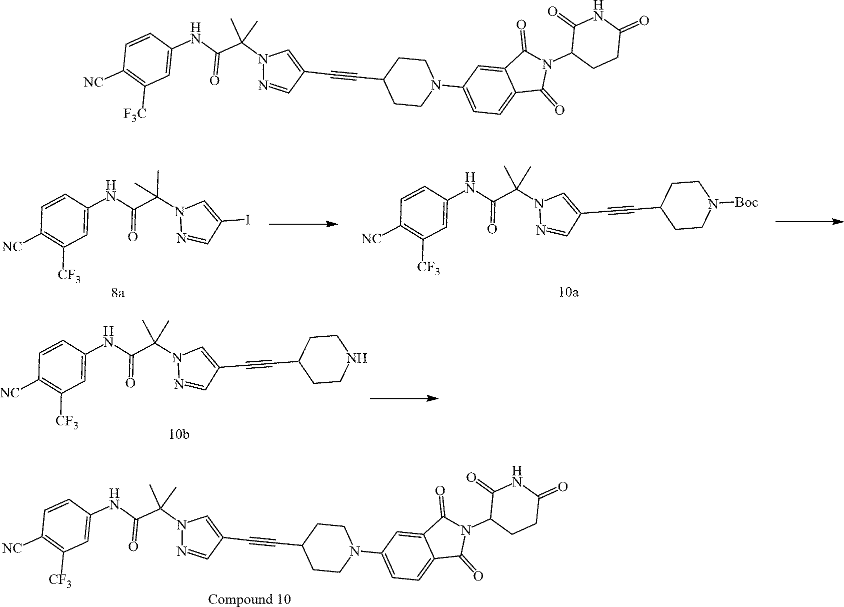

### Step 1: Tert-butyl 4-((1-(1-((4-cyano-3-(trifluoromethyl)phenyl)amino)-2-methyl-1-oxopropan-2-yl)-1H-pyrazol-4-yl)ethynyl)piperidine-1-carboxylate (10a)

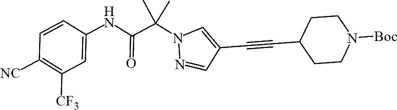

N-cyano-3-(trifluoromethyl)phenyl)-2-iodo-1H-pyrazol-1-yl)-2-methylpropanamide (8a) (224 mg, 0.50 mmol) was dissolved in 10 mL of dichloromethane. Triethylamine (150 mg, 1.48 mmol) was added, PdCl₂(PPh₃)₂ (35 mg, 0.05 mmol) and cuprous iodide (10 mg, 0.05 mmol) were added successively under nitrogen atmosphere, and then a solution of tert-butyl 4-ethynylpiperidine-1-carboxylate (170 mg, 0.81 mmol) in dichloromethane (2 mL) was slowly added dropwise. After the addition was completed, the reaction solution was reacted at room temperature for 16 h. 15 mL of water and 10 mL of dichloromethane were added to the reaction solution. The liquid separation was conducted, and the organic layer was washed with 5 mL of saturated sodium chloride solution, dried over anhydrous sodium sulfate and concentrated under reduced pressure. The residue was separated and purified by silica gel column chromatography (petroleum ether/ethyl acetate (v/v) = 4 : 1) to obtain tert-butyl 4-((1-(1-((4-cyano-3-(trifluoromethyl)phenyl)amino)-2-methyl-1-oxopropan-2-yl)-1H-pyrazol-4-yl)ethynyl)piperidine-1-carboxylate (10a) (200 mg, yield: 76%).

### Step 2: N-(4-cyano-3-(trifluoromethyl)phenyl)-2-methyl-2-(4-(piperidin-4-ylethynyl)-1H-pyrazol-1-yl)propanamide (10b)

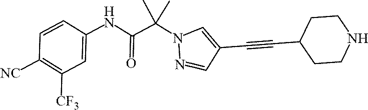

Tert-butyl 4-((1-(1-((4-cyano-3-(trifluoromethyl)phenyl)amino)-2-methyl-1-oxopropan-2-yl)-1H-pyrazol-4-yl)ethynyl)piperidine-1-carboxylate (10a) (0.20 g, 0.38 mmol) was dissolved in 10 mL of DCM. 6 mL of trifluoroacetic acid was added, and the mixture was stirred at room temperature for 3 h. After the reaction was completed, the reaction system was directly concentrated under reduced pressure, and the residue was dissolved in 20 mL of 4 mol/L aqueous NaOH. The reaction solution was extracted with DCM (40 mL × 3). The organic phase was washed with 50 mL of water, dried over anhydrous sodium sulfate and concentrated under reduced pressure to obtain crude N-(4-cyano-3-(trifluoromethyl)phenyl)-2-methyl-2-(4-(piperidin-4-ylethynyl)-1H-pyrazol-1-yl)propanamide (10b) (0.16 g).

$$\text{LCMS} \text{m} / \text{z} = 430.2 {\left[\text{M+1}\right]}^{+}$$

### Step 3: N-(4-cyano-3-(trifluoromethyl)phenyl)-2-(4-((1-(2-(2,6-dioxopiperidin-3-yl)-1,3-dioxoisoindolin-5-yl)piperidin-4-yl)ethynyl)-1H-pyrazol-1-yl)-2-methylpropanamide (Compound 10)

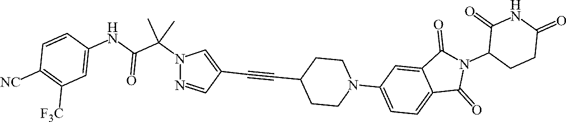

The crude N-(4-cyano-3-(trifluoromethyl)phenyl)-2-methyl-2-(4-(piperidin-4-ylethynyl)-1H-pyrazol-1-yl)propanamide (10b) (0.15 g) was dissolved in10 mL of DMSO. 0.5 mL of DIPEA and 2-(2,6-dioxopiperidin-3-yl)-5-fluoroisoindoline-1,3-dione (see WO 2017197056 for the synthetic method) (145 mg, 0.53 mmol) was added, and the mixture was stirred at 80° C. for 5 h. The reaction solution was cooled to room temperature, and 20 mL of water was added. The mixture was filtered to collect a solid, which was washed with 20 mL of water, dissolved with 50 mL of dichloromethane, dried over anhydrous sodium sulfate and concentrated under reduced pressure, and then the crude product was separated and purified by silica gel column chromatography (dichloromethane/methanol (v/v) = 15 : 1) to obtain N-(4-cyano-3-(trifluoromethyl)phenyl)-2-(4-((1-(2-(2,6-dioxopiperidin-3-yl)-1,3-dioxoisoindolin-5-yl)piperidin-4-yl)ethynyl)-1H-pyrazol-1-yl)-2-methylpropanamide (compound 10) (45 mg, two-step yield calculated from compound 10a: 18%).

¹H NMR (400 MHz, CDC1₃) δ 9.49 (s, 1H), 8.13 (s, 1H), 7.96 - 7.90 (m, 1H), 7.82 - 7.65 (m, 5H), 7.30 (d, 1H), 7.10 (dd, 1H), 4.94 (dd, 1H), 3.78 - 3.68 (m, 2H), 3.39 -3.28 (m, 2H), 2.96 - 2.66 (m, 4H), 2.18 - 1.99 (m, 3H), 1.92 (s, 6H), 1.88 - 1.78 (m, 2H).

$$\text{LCMS} \text{m} / \text{z} = 686.2 {\left[\text{M+1}\right]}^{+}$$

### BIOLOGICAL TEST EXAMPLES

### 1. Experiment on Inhibiting VCaP Cell Proliferation

Prostate cancer cells VCaP were purchased from ATCC, the cell medium was 1640 + 10% FBS, and the cells were cultured in an incubator at 37° C. under 5% CO₂. On day 1, the cells in an exponential phase were collected. With a 1% css-FBS phenol red-free medium, the cell suspension was adjusted to a corresponding concentration, plated into T0 wells at 7500 cells/well and incubated for 3 days. After the incubation was completed, R1881 at a final concentration of 0.1 nM and compounds at different concentrations were added, and the plate was placed in the incubator and incubated for another 7 days. On the day of the addition, T₀ plate was detected using a CellTiter-Glo kit and recorded as RLU₀. After the incubation was completed, 100 µL of mediums was pipetted from each well. According to operation instructions for a CellTiter-Glo kit (Promega, G7573), 50 µL of CellTiter-Glo reagents, which were already pre-melted and equilibrated to room temperature, were then added to each well, and the mixtures were uniformly mixed for 2 min using a microplate shaker. The plate was placed at room temperature for 10 min, and then fluorescence signal values were measured using a microplate reader (PHERAstar FSX). The results were processed according to formula (1), the inhibition rate of the compound at different concentrations was calculated, and the GI₅₀ value of the compound with an inhibition rate of 50% was calculated using origin9.2 software, wherein RLU _{compound} was the readout of the treated group, and RLU_{control} was the average value of the solvent control group.

$$\begin{matrix}\text{Inhibition \% =} \\ \left[1 \text{-} \left({\text{RLU}}_{\text{compound}} {\text{- RLU}}_{0}\right) / \left({\text{RLU}}_{\text{control}} {\text{- RLU}}_{0}\right)\right] \times 100 \%\end{matrix} \qquad \text{formula (1)}$$

The results for GI₅₀ values on proliferation inhibition of VCaP cells were shown in Table 1.

TABLE 1

GI₅₀ values for inhibition of VCaP cells by compounds of the present disclosure

| Serial No. | Compound No. | GI₅₀ (µM) |
|---|---|---|
| 1 | Compound 2 | 4.79 |
| 2 | Compound 3 | 3.09 |
| 3 | Compound 4 | 1.81 |
| 4 | Compound 5 | 1.91 |
| 5 | Compound 6 | 1.69 |
| 6 | Compound 7 | 0.47 |
| 7 | Compound 8 | 3.96 |
| 8 | Compound 9 | 0.18 |
| 9 | Compound 10 | 0.33 |

Conclusion: the compounds of the present disclosure had an inhibition effect on prostate cancer cells VCaP.

### 2. Pharmacokinetic Experiment in Rats

Objective: in this experiment, a single dose of each test compound was administered to SD rats intravenously and intragastrically, the concentrations of the test compound in plasma of rats were measured, and the pharmacokinetic characteristics and bioavailability of the test compound in rats were evaluated.

Experimental animals: male SD rats, 200-250 g, 6-8 weeks old, 6 rats/compound, purchased from CHENGDU DOSSY EXPERIMENTAL ANIMALS CO., LTD.

Experimental method: on the day of the experiment, six SD rats were randomly grouped according to their body weights. The animals were fasted but with water available for 12 to 14 hours one day before the administration, and were fed 4 h after the administration.

TABLE 2

| Group | No. of rats | Administration information | | | | | | |
|---|---|---|---|---|---|---|---|---|
| | Male | Test compound | Administr ation dosage* (mg/kg) | Administr ation concentrat ion (mg/mL) | Administr ation volume (mL/kg) | Collected samples | Mode of administ ration | Vehicle |
| G1 | 3 | Compound of the present disclosure | 5 | 1 | 5 | Plasma | Intraven ously | 5% DMA + 5% Solutol + 90% Saline |
| G2 | 3 | Compound of the present disclosure | 20 | 2 | 10 | Plasma | Oral (intragas trically) | 5% DMSO+ 30% PEG-400 + 65% (20% SBE-(β-CD) |

*Dosage is calculated based on free base.

Sampling: before and after the administration, 0.1 mL of blood was taken from the orbit of the rats under isoflurane anesthesia and placed in an EDTAK2 centrifuge tube. Centrifugation was carried out at 5000 rpm at 4° C. for 10 min, and the plasma was collected.

Time points for plasma collection in G1 and G2 groups: 0, 5 min, 15 min, 30 min, 1 h, 2 h, 4 h, 6 h, 8 h, and 24 h.

Before analysis and detection, all samples were stored at -80° C. The samples were analyzed quantitatively by LC-MS/MS.

TABLE 3

Pharmacokinetic parameters of compounds of the present disclosure in plasma of rats

| Test compounds | Mode of administration* | AUC₀₋ₜ (ng/mL·h) | T_{1/2} (h) | Oral bioavailability |
|---|---|---|---|---|
| Compound 9 | i.g. (20 mg/kg) | 45604 ± 3725 | 24.0 ± 10 | Yes |

*Note: i.g. (intragastrical) administration;

Conclusion: the compounds the present disclosure had a certain oral bioavailability in rats.

### 3. Experiment on Inhibiting 22RV1 Cell Proliferation

Prostate cancer cells 22RV1 were purchased from ATCC, the cell medium was RPMI 1640 + 10% FBS, and the cells were cultured in an incubator at 37° C. under 5% CO₂. On day 1, the cells in an exponential phase were collected. With a 1% css-FBS phenol red-free medium, the cell suspension was adjusted to a corresponding concentration, plated at 2000 cells/well and incubated overnight. On day 2, compounds at different concentrations were added, and the plate was placed in the incubator and incubated for another 7 days. After the incubation was completed, according to operation instructions for a CellTiter-Glo kit (Promega, G7573), 50 µL of CellTiter-Glo reagents, which were already pre-melted and equilibrated to room temperature, were added to each well, and the mixtures were uniformly mixed for 2 min using a microplate shaker. The plate was placed at room temperature for 10 min, and then fluorescence signal values were measured using a microplate reader (PHERAstar FSX). The results were processed according to formula (2), the inhibition rate of the compound at different concentrations was calculated, and the IC₅₀ value of the compound with an inhibition rate of 50% was calculated using origin9.2 software with DoseResp function, wherein RLU_{compound} was the readout of the treated group, and RLU_{control} was the average value of the DMSO solvent control group.

$$\text{Inhibition \% =} \left[1 \text{-} {\text{RLU}}_{\text{compound}} / {\text{RLU}}_{\text{control}}\right] \times 100 \% \qquad \text{formula (2)}$$

The results for IC₅₀ values on proliferation inhibition of 22RV1 cells were shown in Table 4.

TABLE 4

IC50 values for inhibition of 22RV1 cells by compounds of the present disclosure

| Serial No. | Compound No. | IC₅₀ (µM) |
|---|---|---|
| 1 | Compound 9 | 0.078 |

Conclusion: the compounds of the present disclosure had an inhibition effect on prostate cancer cells 22RV1.

### 4. Degradation Experiment on Full-Length AR (AR-FL) and AR Splice Variants (AR-Vs) in 22RV1 Cells

Prostate cancer cells 22RV1 were purchased from ATCC, the cell medium was 1640 + 10% FBS, and the cells were cultured in an incubator at 37° C. under 5% CO₂. On day 1, the cells in an exponential phase were collected. With a 1% css-FBS phenol red-free medium, the cell suspension was adjusted to a corresponding concentration and plated into a 6-well plate at 1 mL/well and 100000 cells/ well. The next day, a 1% css-FBS phenol-free medium containing the compound to be tested was added, a 1% css-FBS phenol-free medium containing 0.2% DMSO was added to one well as DMSO solvent control, and the 6-well plate was cultured in an incubator at 37° C. under 5% CO₂. After 24 h, the cells were digested with trypsin and collected into a 1.5 mL centrifuge tube. 15 µL of RIPA lysate (containing 1X protease inhibitor cocktail) was added to each well, and the cells were lysed on ice for 15 min and then centrifuged at 12000 g at 4° C. for 10 min. The supernatant protein samples were collected, and protein quantification was performed by BCA method. AR-FL and AR-Vs were detected using fully automatic protein expression quantitative analysis, which involved diluting the protein to be tested to 1 mg/mL. 4 µL of the diluted protein samples were added to 1 µL of 5× Master Mix (provided in the kit), and the prepared samples were denatured at 95° C. for 5 min and placed on ice for use. The primary antibodies, AR (CST, 5153S) and β-actin (CST, 3700), were diluted with Antibody Diluent II (provided in the kit) at a dilution ratio of 1 : 20 and 1 : 200, respectively. The secondary antibody was a mixed goat anti-mouse and goat anti-rabbit secondary antibody in a ratio of 1 : 1, and the color development solution was a mixed solution of Lumino-S and Peroxide in a ratio of 1 : 1. The prepared reagents were successively added to an assay plate according to instructions of the kit and detected. Western blot band processing was performed using “Compass for SW”, a fully automatic protein expression quantitative analysis software, in which western blot bands were automatically simulated based on signal values. The degradation rate of AR-FL (1) or AR-Vs (2) relative to solvent control at different compound concentrations was calculated according to formulas (1) and (2), wherein AR-FL_{compound} was the relative peak area of AR-FL in the treated group, and AR-FLₛₒₗᵥₑₙₜ was the relative peak area of AR-FL in the solvent control group. AR-Vs_{compound} was the relative peak area of AR-Vs in the treated group, and AR-Vsₛₒₗᵥₑₙₜ was the relative peak area of AR-Vs in the solvent control group.

$$\begin{matrix}\text{Degradation rate of AR-FL=} \\ \left(1 {\text{- AR-FL}}_{\text{compound}} / {\text{AR-FL}}_{\text{solvent}}\right) \times 100 \%\end{matrix} \qquad \text{formula (1)}$$

$$\begin{matrix}\text{Degradation rate of AR-Vs=} \\ \left(1 \text{-} {\text{AR-Vs}}_{\text{compound}} / {\text{AR-Vs}}_{\text{solvent}}\right) \times 100 \%\end{matrix} \qquad \text{formula (2)}$$

DC₅₀ calculation: the compound concentration DC₅₀ values at which the degradation rate of AR-FL or AR-Vs was 50% were calculated using OriginPro2015 software and analyzed using DoseResp function according to formula (1) or (2).

Conclusion: the compounds of the present disclosure had a certain degradation effect on AR-FL or AR-Vs in prostate cancer cells 22RV1.

What is claimed is:

1. A compound or a stereoisomer, a deuterated compound, a solvate, a prodrug, a metabolite, a pharmaceutically acceptable salt or a co-crystal thereof, wherein the compound is selected from a compound as shown in general formula (I), 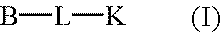
L is selected from -Ak1-Cy1-Ak2-Cy2-Ak3-Cy3-Ak4-Cy4-Ak5-;
Ak1, Ak2, Ak3, Ak4 and Ak5 are each independently selected from CH₂, O, C≡C or a bond;
Cy1, Cy2, Cy3 and Cy4 are each independently selected from a bond, a 4- to 7-membered mono-heterocyclic ring, a 5- to 10-membered fused heterocyclic ring, a 6- to 12-membered spiro-heterocyclic ring, a 7- to 10-membered bridged-heterocyclic ring, 4-to 7-membered monocycloalkyl, 5- to 10-membered fused cycloalkyl, 6- to 12-membered spiro cycloalkyl, 7- to 10-membered bridged cycloalkyl, 5- to 10-membered heteroaryl or 6- to 10-membered aryl, wherein the aryl, heteroaryl, cycloalkyl, mono-heterocyclic ring, fused heterocyclic ring, spiro-heterocyclic ring or bridged-heterocyclic ring is optionally further substituted with 0 to 4 substituents selected from H, F, Cl, Br, I, OH, COOH, CN, NH₂, oxo, C₁₋₄ alkyl, halogen-substituted C₁₋₄ alkyl, hydroxyl-substituted C₁₋₄ alkyl or C₁₋₄ alkoxy, and the heteroaryl, mono-heterocyclic ring, fused heterocyclic ring, spiro-heterocyclic ring or bridged-heterocyclic ring contains 1 to 4 heteroatoms selected from O, S and N;
B is selected from 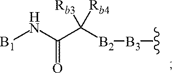
B₁ is selected from one of the following substituted or unsubstituted groups: 6-membered aryl or 6-membered heteroaryl, which, when substituted, is optionally further substituted with 0 to 4 R^{b1}, wherein the heteroaryl contains 1 to 4 heteroatoms selected from O, S and N;
B₂ is selected from one of the following substituted or unsubstituted groups: a 5- to 10-membered heterocyclic group or —NHC(═O)—, which, when substituted, is optionally further substituted with 0 to 4 R^{b2}, wherein the heterocyclic group contains 1 to 4 heteroatoms selected from O, S and N;
B₃ is selected from substituted or unsubstituted 5- to 6-membered aryl or a bond, wherein the 5- to 6-membered aryl, when substituted, is optionally further substituted with 0 to 4 R^{b2};
R^{b1} and R^{b2} are each independently selected from H, F, Cl, Br, I, OH, NH₂, CN, CF₃, —C(═O)NH₂, -C(=O)NH-C₁₋₄ alkyl, —C(═O)N(C₁₋₄ alkyl)₂, C₁₋₄ alkyl or C₁₋₄ alkoxy, wherein the alkyl or alkoxy is optionally further substituted with 0 to 4 substituents selected from H, F, Cl, Br, I or OH;
R^{b3} and R^{b4} are each independently selected from H or C₁₋₆ alkyl, wherein the alkyl is optionally further substituted with 0 to 4 substituents selected from H, F, Cl, Br, I, OH, NH₂, C₁₋₄ alkyl or C₁₋₄ alkoxy;
or R^{b3} and R^{b4} together with the carbon atoms to which they are attached form C₃₋₆ cycloalkyl or a C₃₋₆ mono-heterocyclic ring, wherein the cycloalkyl or mono-heterocyclic ring is optionally further substituted with 0 to 4 substituents selected from H, F, Cl, Br, I, OH, NH₂, C₁₋₄ alkyl or C₁₋₄ alkoxy, and the mono-heterocyclic ring contains 1 to 4 heteroatoms selected from O, S and N;
K is selected from 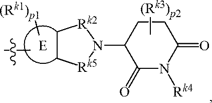 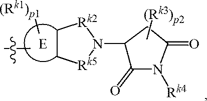 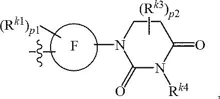 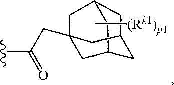 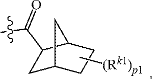 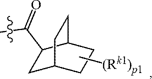 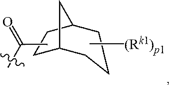 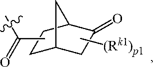 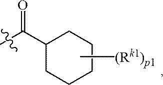 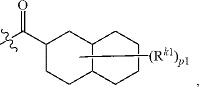 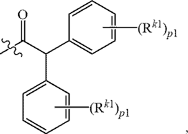 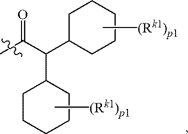 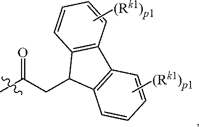 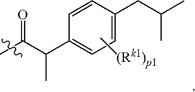 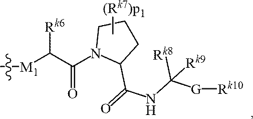 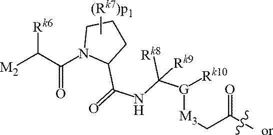 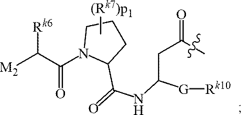
ring E or F is each independently selected from a phenyl ring or a 5- to 6-membered heteroaryl ring, wherein the heteroaryl ring contains 1 to 2 heteroatoms selected from O, S and N;
each R^{k2} is independently selected from CH₂, C═O, S═O and SO₂;
R^{k1}, R^{k3} or R^{k4} is each independently selected from H, F, Cl, Br, I, OH, NH₂, CF₃, CN, COOH, C₁₋₄ alkyl or C₁₋₄ alkoxy;
R^{k5} is selected from C═O or 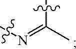
M₁ is selected from a bond, —CH₂—C(═O)NH— or —C(═O)CH₂NH—;
M₂ is selected from -NHC(=O)-C₁₋₆ alkyl or -NHC(=O)-C₃₋₆ cycloalkyl, wherein the alkyl or cycloalkyl is optionally further substituted with 0 to 4 substituents selected from H, F, Cl, Br, I, OH, NH₂, C₁₋₄ alkyl or C₁₋₄ alkoxy;
M₃ is selected from —NH— or —O—;
R^{k6} is selected from C₁₋₆ alkyl, wherein the alkyl is optionally further substituted with 0 to 4 substituents selected from H, F, Cl, Br, I, OH, C₁₋₆ alkyl or C₃₋₆ cycloalkyl;
each R^{k7} is independently selected from H, F, Cl, Br, I, OH, SH, C₁₋₆ alkyl, C₁₋₆ alkoxy, C₁₋₆ alkylthio or C₁₋₆ alkylformyloxy, wherein the alkyl, alkoxy or alkylthio is optionally further substituted with 0 to 4 substituents selected from H, F, Cl, Br, I, OH, C₁₋₄ alkyl or C₁₋₄ alkoxy;
R^{k8} and R^{k9} are each independently selected from H, C₁₋₆ alkyl or C₃₋₆ cycloalkyl, wherein the alkyl or cycloalkyl is optionally further substituted with 0 to 4 substituents selected from H, F, Cl, Br, I, OH, NH₂, C₁₋₄ alkyl or C₁₋₄ alkoxy;
R^{k10} is selected from 5- to 6-membered heteroaryl, wherein the heteroaryl is optionally further substituted with 0 to 4 substituents selected from H, F, Cl, Br, I, OH, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl;
G is selected from 6- to 10-membered aryl or 5- to 10-membered heteroaryl, wherein the aryl or heteroaryl is optionally further substituted with 0 to 4 substituents selected from H, F, Cl, Br, I, OH, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl; and
p1 or p2 is each independently selected from 0, 1, 2, 3 or 4.

2. The compound or the stereoisomer, deuterated compound, solvate, prodrug, metabolite, pharmaceutically acceptable salt or co-crystal thereof according to claim 1, wherein
Cy1, Cy2, Cy3 and Cy4 are each independently selected from a bond, a 4- to 7-membered nitrogen-containing mono-heterocyclic ring, a 5- to 10-membered nitrogen-containing fused heterocyclic ring, a 7- to 10-membered bridged-heterocyclic ring, or a 6- to 12-membered nitrogen-containing spiro-heterocyclic ring, wherein the mono-heterocyclic ring, fused heterocyclic ring, bridged-heterocyclic ring or spiro-heterocyclic ring is optionally further substituted with 0 to 4 substituents selected from H, F, Cl, Br, I, OH, COOH, CN, NH₂, oxo, C₁₋₄ alkyl, halogen-substituted C₁₋₄ alkyl, hydroxyl-substituted C₁₋₄ alkyl or C₁₋₄ alkoxy, and the mono-heterocyclic ring, fused heterocyclic ring, bridged-heterocyclic ring or spiro-heterocyclic ring contains 1 to 4 heteroatoms selected from O, S and N; and
K is selected from 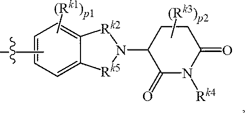 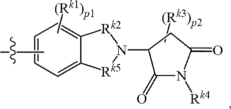 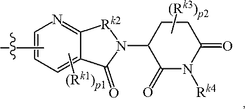 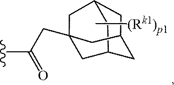 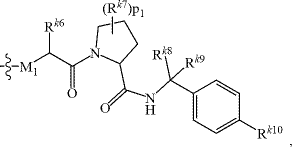 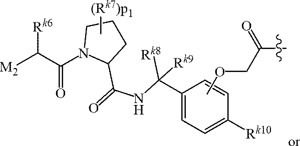 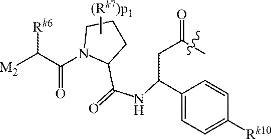
.

3. The compound or the stereoisomer, deuterated compound, solvate, prodrug, metabolite, pharmaceutically acceptable salt or co-crystal thereof according to claim 2, wherein
Cy1, Cy2, Cy3 and Cy4 are each independently selected from a bond or one of the following substituted or unsubstituted groups: azetidinyl, azacyclopentyl, azacyclohexyl, piperidine, morpholine, piperazine, cyclopropyl-fused-azetidinyl, cyclopropyl-fused-azacyclopentyl, cyclopropyl-fused-azacyclohexyl, cyclopropyl-fused-piperidine, cyclobutyl-fused-azetidinyl, cyclobutyl-fused-azacyclopentyl, cyclobutyl-fused-azacyclohexyl, cyclobutyl-fused-piperidine, cyclopentyl-fused-azetidinyl, cyclopentyl-fused-azacyclopentyl, cyclopentyl-fused-azacyclohexyl, cyclopentyl-fused-piperidine, cyclohexyl-fused-azetidinyl, cyclohexyl-fused-azacyclopentyl, cyclohexyl-fused-azacyclohexyl, cyclohexyl-fused-piperidine, azetidinyl-fused-azetidinyl, azetidinyl-fused-azacyclopentyl, azetidinyl-fused-azacyclohexyl, azetidinyl-fused-piperidine, azacyclopentyl-fused-azetidinyl, azacyclopentyl-fused-azacyclopentyl, azacyclopentyl-fused-azacyclohexyl, azacyclopentyl-fused-piperidine, azacyclohexyl-fused-azetidinyl, azacyclohexyl-fused-azacyclopentyl, azacyclohexyl-fused-azacyclohexyl, azacyclohexyl-fused-piperidine, cyclobutyl-spiro-azetidinyl, cyclobutyl-spiro-azacyclopentyl, cyclobutyl-spiro-azacyclohexyl, cyclopentyl-spiro-azetidinyl, cyclopentyl-spiro-azacyclopentyl, cyclopentyl-spiro-azacyclohexyl, cyclohexyl-spiro-azetidinyl, cyclohexyl-spiro-azacyclopentyl, cyclohexyl-spiro-azacyclohexyl, azetidinyl-spiro-azetidinyl, azetidinyl-spiro-azacyclopentyl, azetidinyl-spiro-azacyclohexyl, azacyclopentyl-spiro-azetidinyl, azacyclopentyl-spiro-azacyclopentyl, azacyclopentyl-spiro-azacyclohexyl, azacyclohexyl-spiro-azetidinyl, azacyclohexyl-spiro-azacyclopentyl, azacyclohexyl-spiro-azacyclohexyl, cyclobutyl-spiro-piperidine, cyclopentyl-spiro-piperidine, cyclohexyl-spiro-piperidine, azetidinyl-spiro-piperidine, azacyclopentyl-spiro-piperidine, azacyclohexyl-spiro-piperidine, 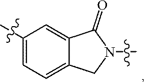 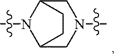 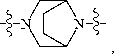 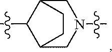 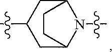 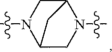 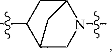 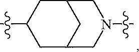 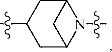 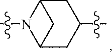 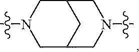 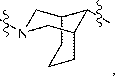 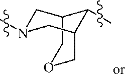 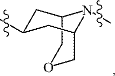
which, when substituted, is optionally further substituted with 0 to 4 substituents selected from H, F, Cl, Br, I, OH, NH
₂, COOH, CN, oxo, C₁₋₄ alkyl, halogen-substituted C₁₋₄ alkyl, hydroxyl-substituted C₁₋₄ alkyl or C₁₋₄ alkoxy;
R^{b1} and R^{b2} are each independently selected from H, F, Cl, Br, I, OH, NH₂, CN, CF₃, —C(═O)NH₂, —C(═O)NH— CH₃, —C(═O)N(CH₃)₂, methyl, ethyl, propyl, isopropyl, methoxy or ethoxy, wherein the methyl, ethyl, propyl, isopropyl, methoxy or ethoxy is optionally further substituted with 0 to 4 substituents selected from H, F, Cl, Br, I or OH;
K is selected from 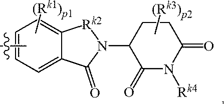 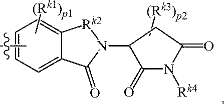 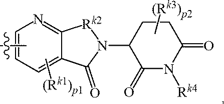 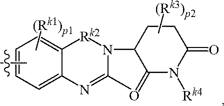 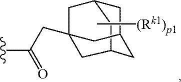 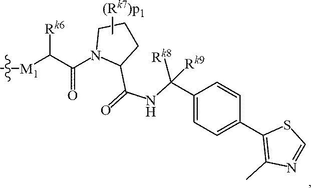 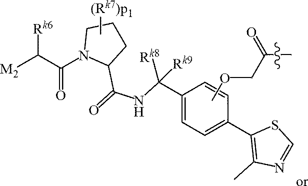 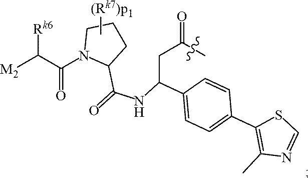
each R^{k2} is independently selected from CH₂ or C═O;
R^{k1}, R^{k3} or R^{k4} is each independently selected from H, CH₃, F, Cl, Br, I, OH or NH₂;
M₁ is selected from a bond, —CH₂—C(═O)NH— or —C(═O)CH₂NH—;
M₂ is selected from -NHC(=O)-methyl, -NHC(=O)-ethyl, -NHC(=O)-cyclopropyl, NHC(=O)-cyclobutyl,NHC(=O)-cyclopentyl or -NHC(=O)-cyclohexyl, wherein the methyl, ethyl, cyclopropyl, cyclobutyl, cyclopentyl or cyclohexyl is optionally further substituted with 0 to 4 substituents selected from H, F, Cl, Br, I, OH, NH₂, C₁₋₄ alkyl or C₁₋₄ alkoxy;
R^{k6} is selected from methyl, ethyl, propyl, isopropyl, tert-butyl, isobutyl or sec-butyl;
each R^{k7} is independently selected from H, F, OH, SH, methyl, methoxy or -SCH₃;
R^{k8} and R^{k9} are each independently selected from H, methyl, ethyl, cyclopropyl or cyclobutyl; and
p1 or p2 is each independently selected from 0, 1 or 2.

4. The compound or the stereoisomer, deuterated compound, solvate, prodrug, metabolite, pharmaceutically acceptable salt or co-crystal thereof according to claim 3, wherein
Cy1, Cy2, Cy3 and Cy4 are each independently selected from one of the following substituted or unsubstituted groups: a bond, 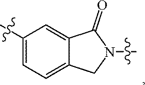 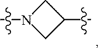 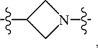 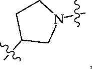 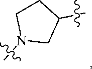 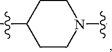 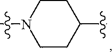 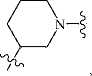 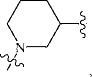 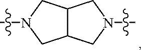 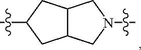 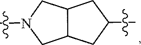 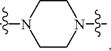 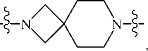 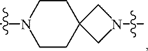 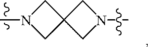 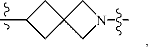 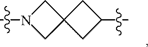 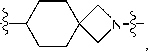 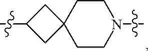 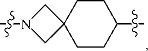 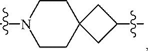 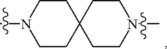 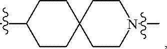 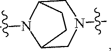 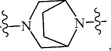 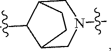 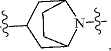 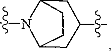 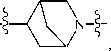 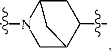 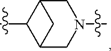 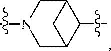 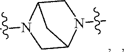 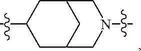 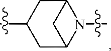 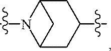 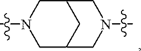 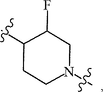 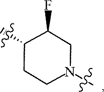 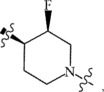 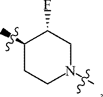 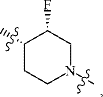 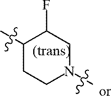 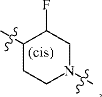
which, when substituted, is optionally further substituted with 0 to 4 substituents selected from H, F, CF
₃, methyl, oxo, hydroxymethyl, COOH, CN or NH₂;
B is selected from 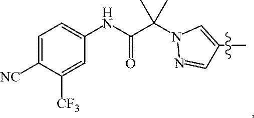 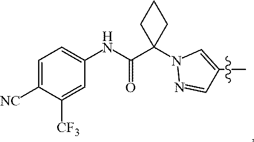 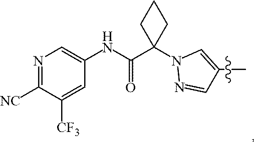 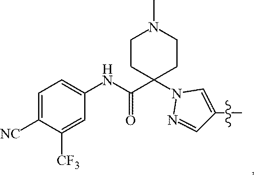 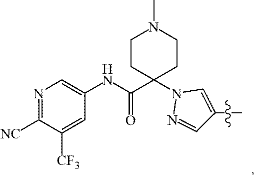 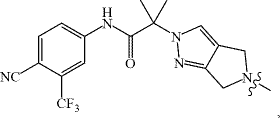 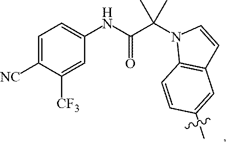 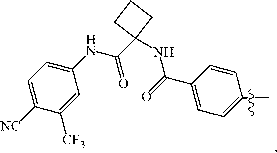 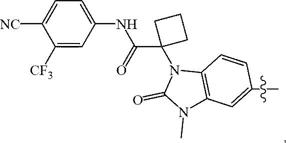 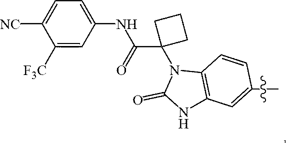 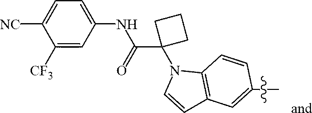 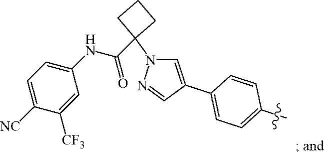
K is selected from 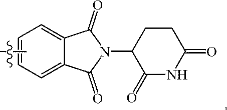 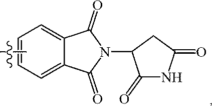 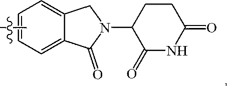 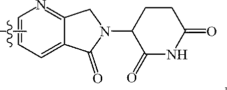 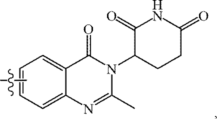 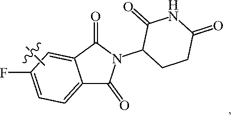 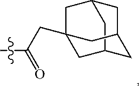 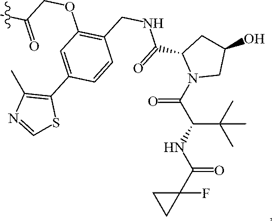 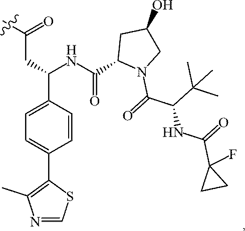 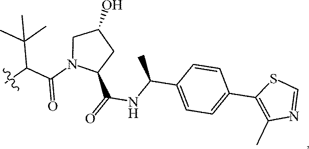 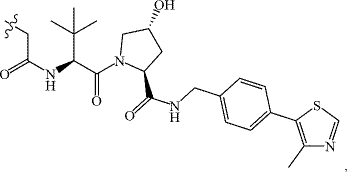 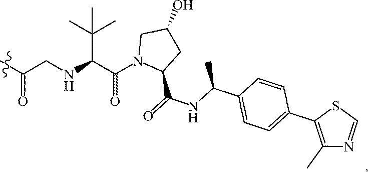 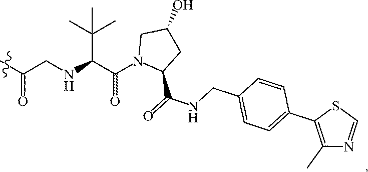 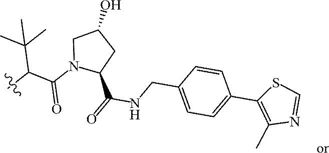 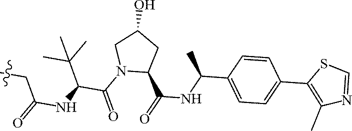
.

5. The compound or the stereoisomer, deuterated compound, solvate, prodrug, metabolite, pharmaceutically acceptable salt or co-crystal thereof according to claim 4, wherein
L is selected from a bond, 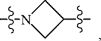 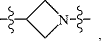 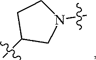 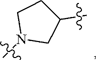 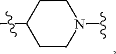 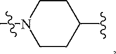 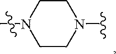 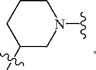 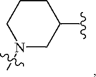 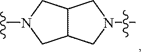 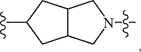 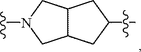 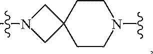 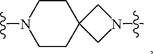 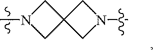 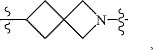 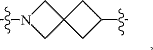 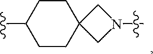 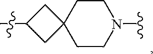 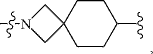 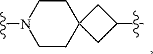 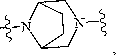 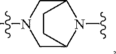 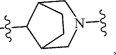 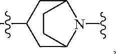 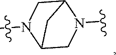 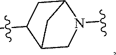 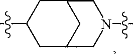 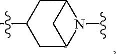 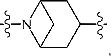 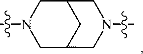 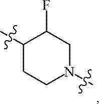 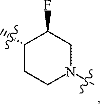 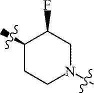 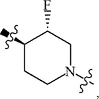 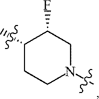 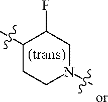 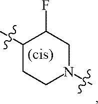
wherein the left side of L is linked to B;
or L is selected from 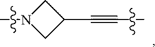 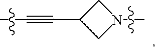 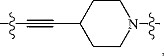 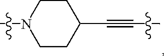 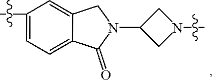 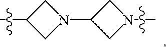 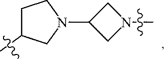 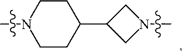 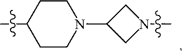 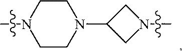 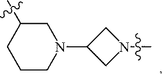 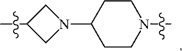 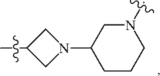 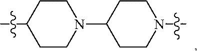 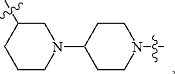 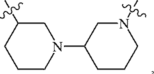 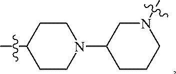 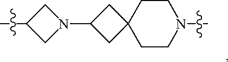 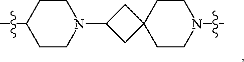 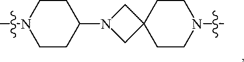 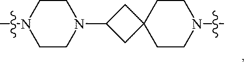 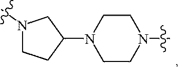 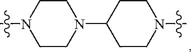 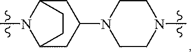 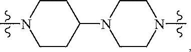 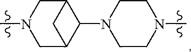 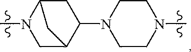 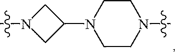 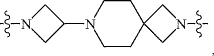 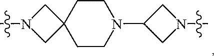 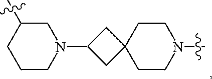 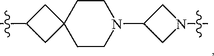 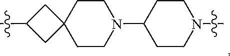 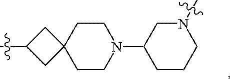 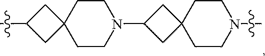 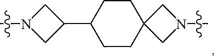 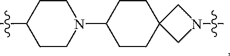 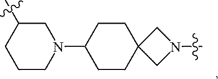 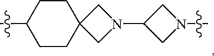 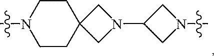 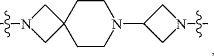 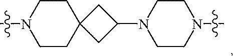 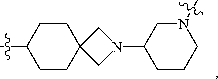 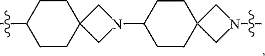 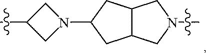 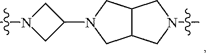 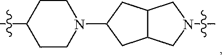 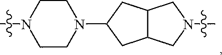 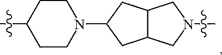 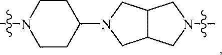 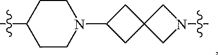 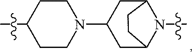 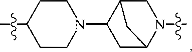 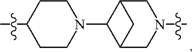 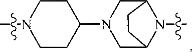 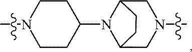 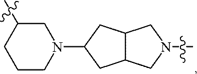 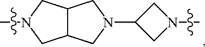 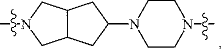 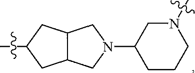 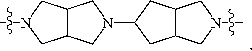 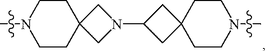 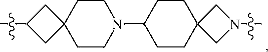 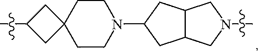 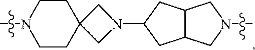 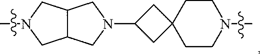 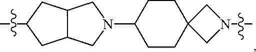 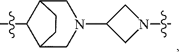 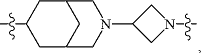 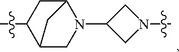 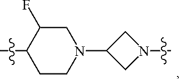 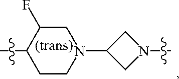 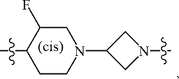 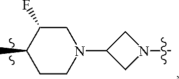 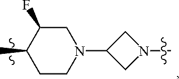 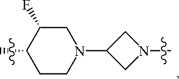 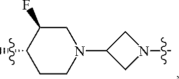 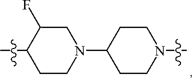 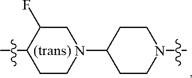 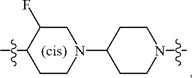 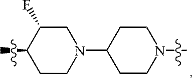 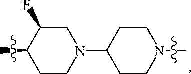 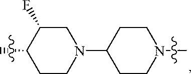 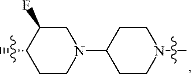 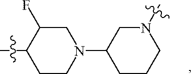 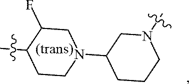 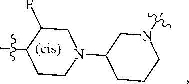 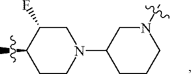 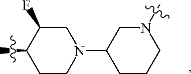 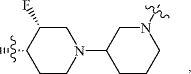 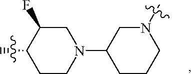 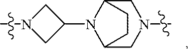 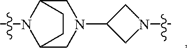 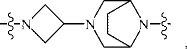 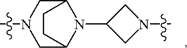 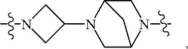 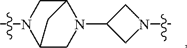 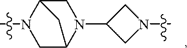 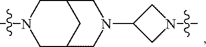 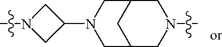 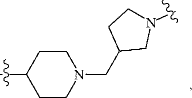
wherein the left side of L is linked to B;
or L is selected from 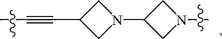 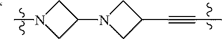 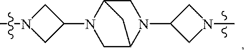 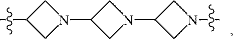 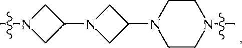 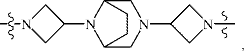 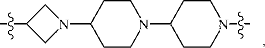 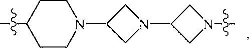 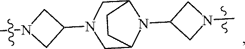 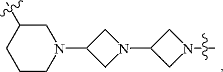 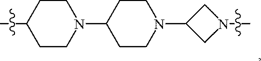 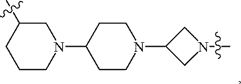 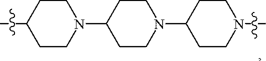 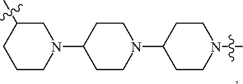 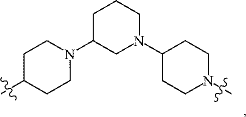 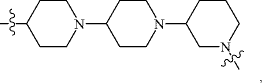 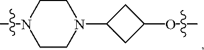 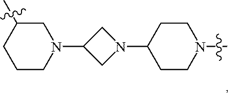 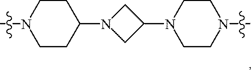 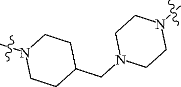 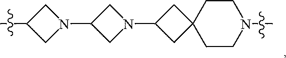 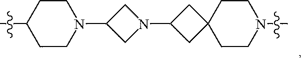 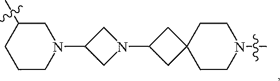 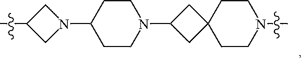 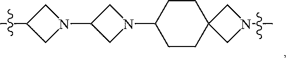 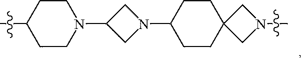 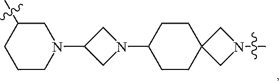 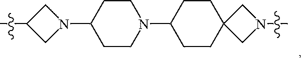 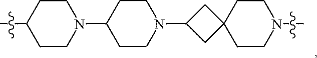 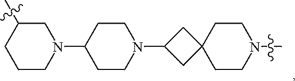 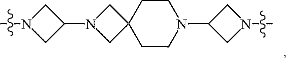 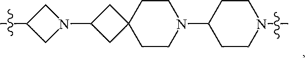 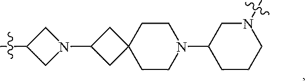 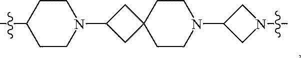 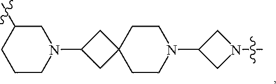 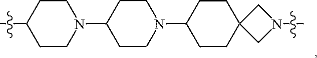 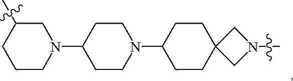 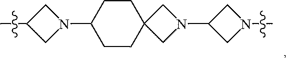 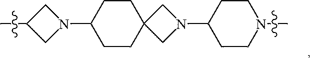 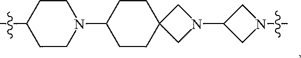 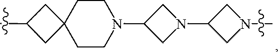 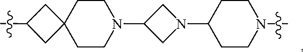 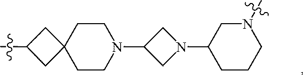 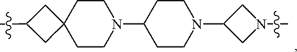 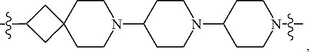 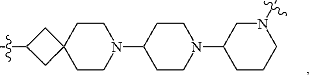 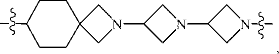 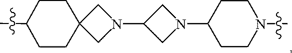 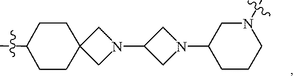 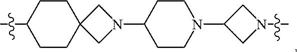 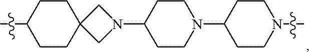 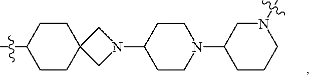 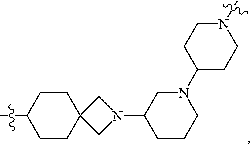 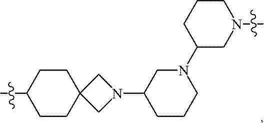 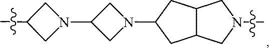 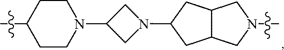 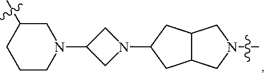 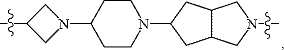 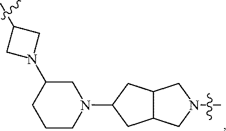 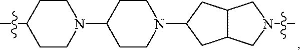 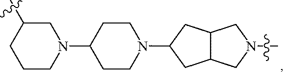 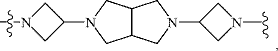 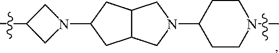 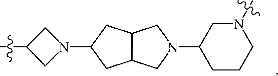 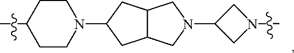 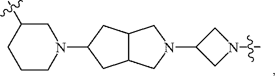 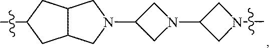 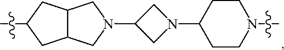 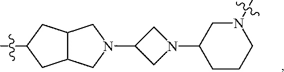 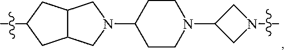 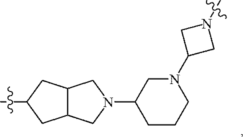 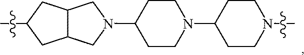 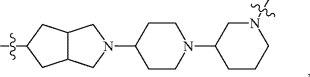 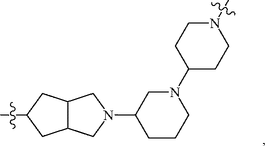 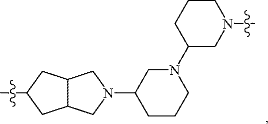 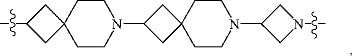 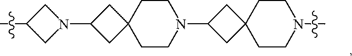 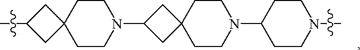 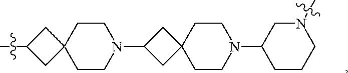 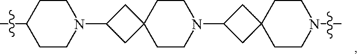 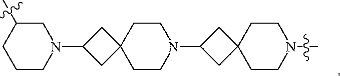 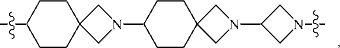 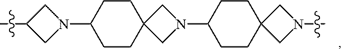 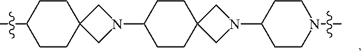 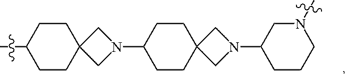 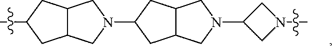 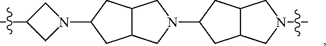 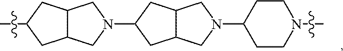 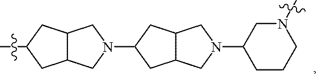 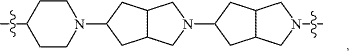 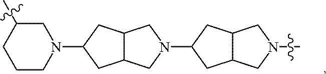 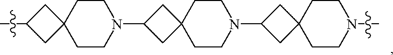 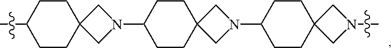 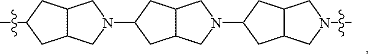 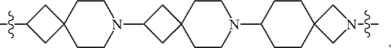 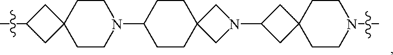 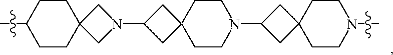 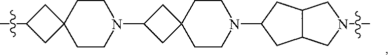 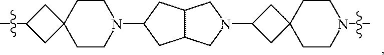 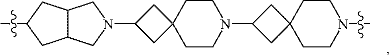 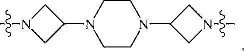 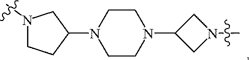 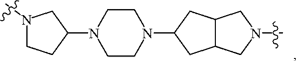 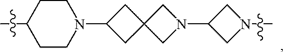 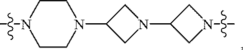 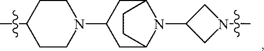 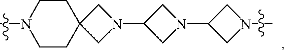 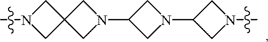 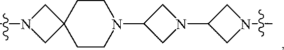 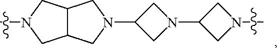 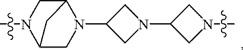 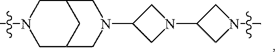 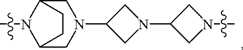 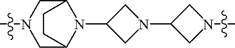 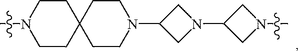 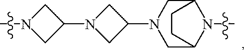 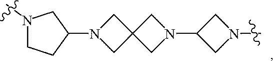 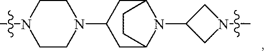 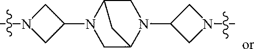 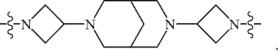
wherein the left side of L is linked to B;
or L is selected from 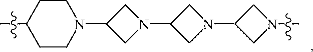 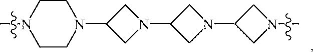 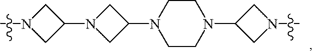 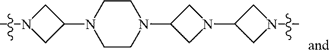 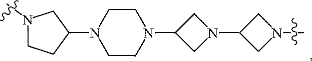
wherein the left side of L is linked to B.

6. The compound or the stereoisomer, deuterated compound, solvate, prodrug, metabolite, pharmaceutically acceptable salt or co-crystal thereof according to claim 5, wherein
K is selected from 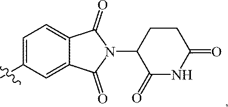 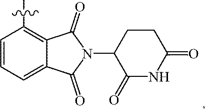 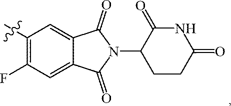 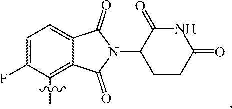 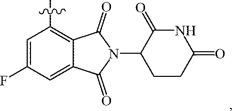 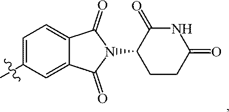 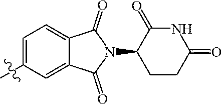 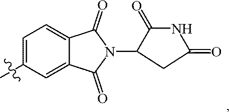 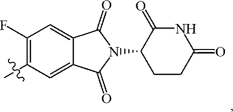 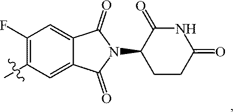 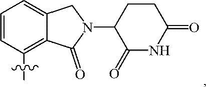 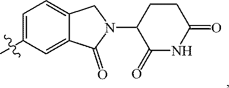 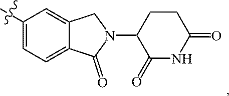 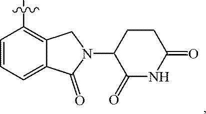 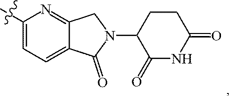 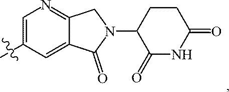 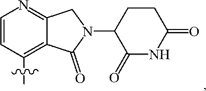 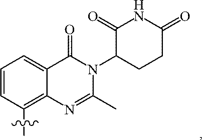 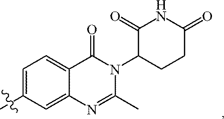 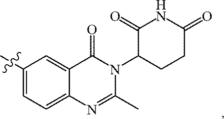 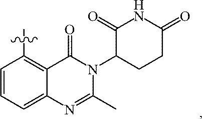 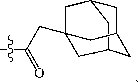 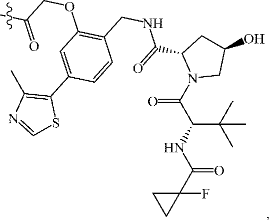 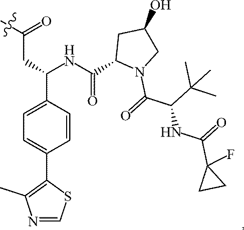 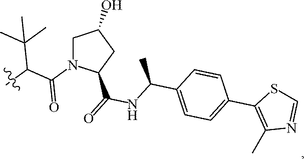 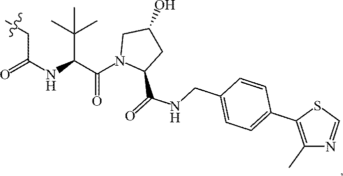 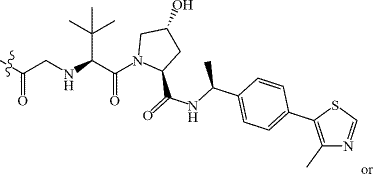 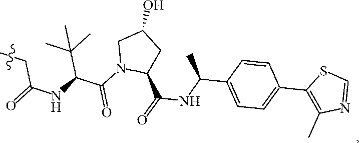
.

7. The compound or the stereoisomer, deuterated compound, solvate, prodrug, metabolite, pharmaceutically acceptable salt or co-crystal thereof according to claim 5, wherein
K is selected from 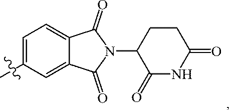 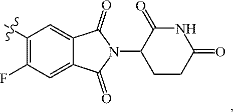 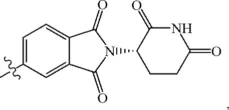 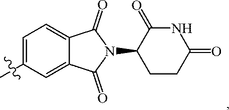 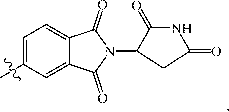 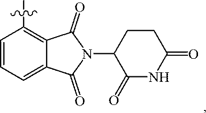 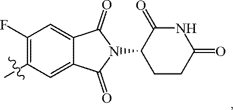 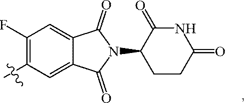 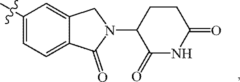 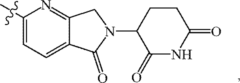 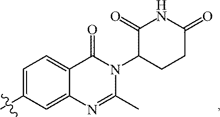 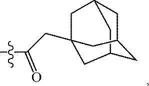 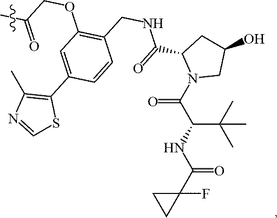 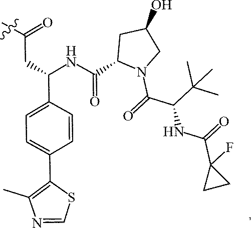 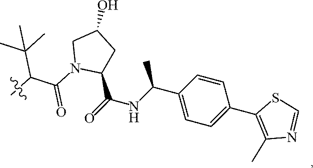 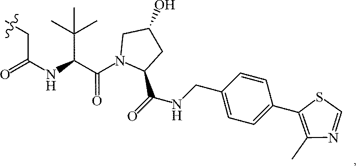 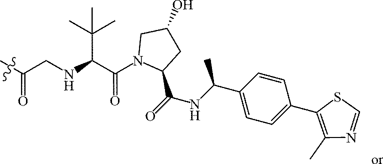 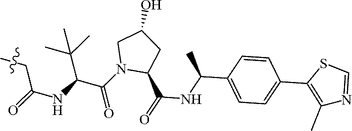
.

8. The compound or the stereoisomer, deuterated compound, solvate, prodrug, metabolite, pharmaceutically acceptable salt or co-crystal thereof according to claim 7, wherein the compound has a structure selected from one of the following structures:

| | |
|---|---|
| 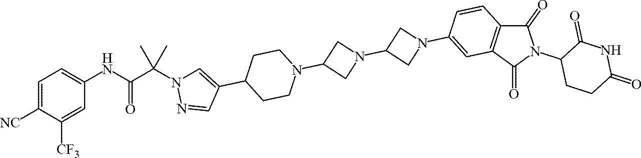 | 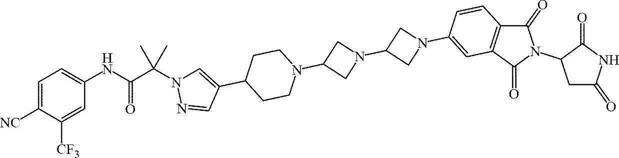 |
| 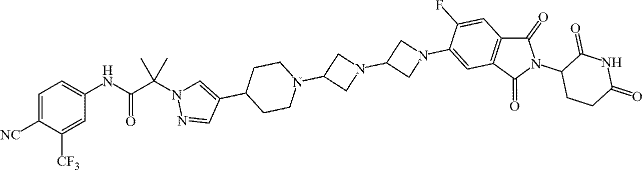 | 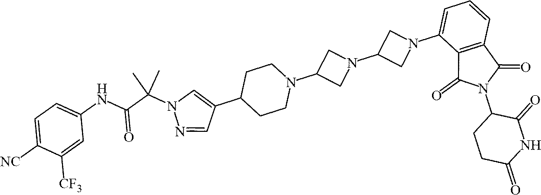 |
| 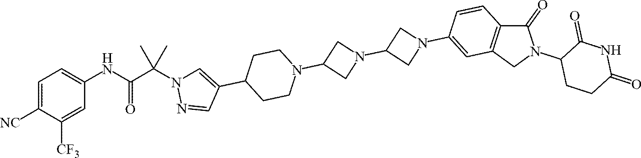 | 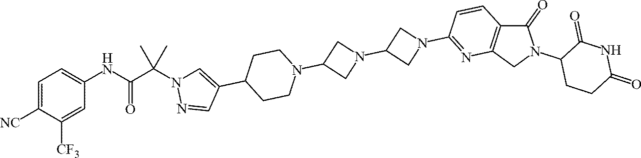 |
| 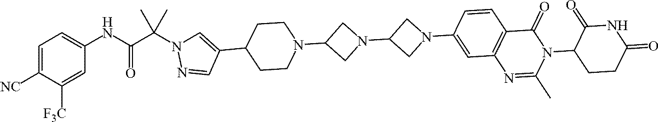 | 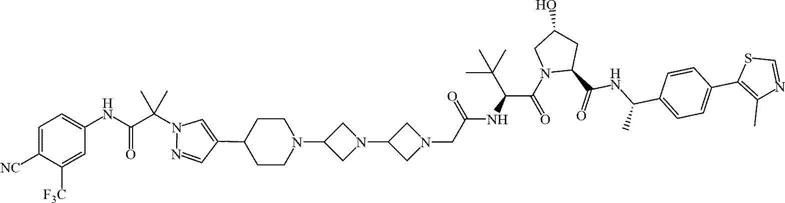 |
| 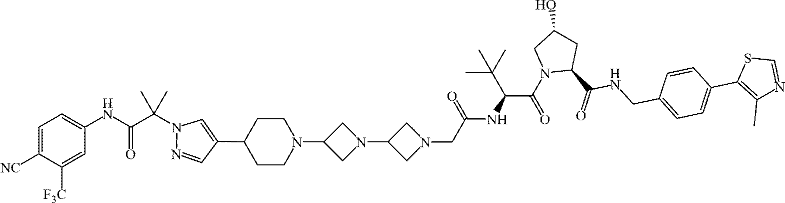 | 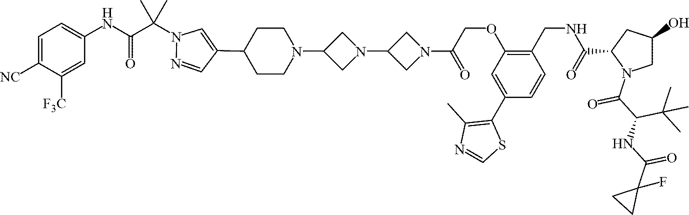 |
| 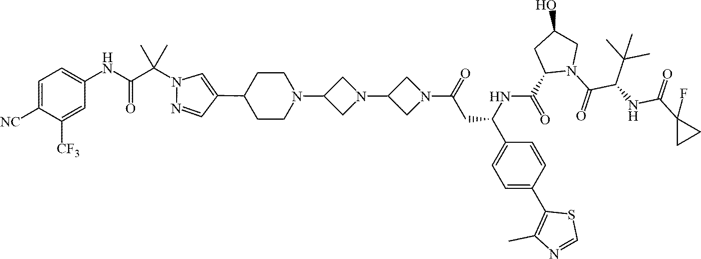 | 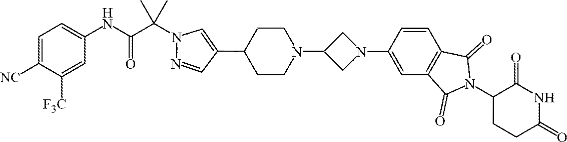 |
| 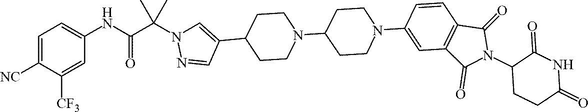 | 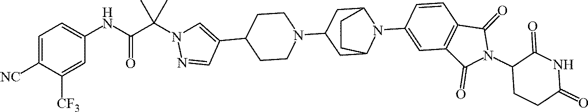 |
| 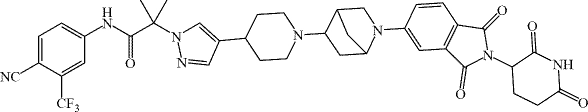 | 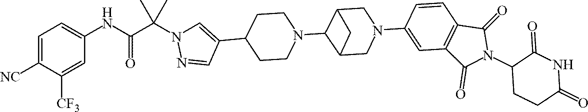 |
| 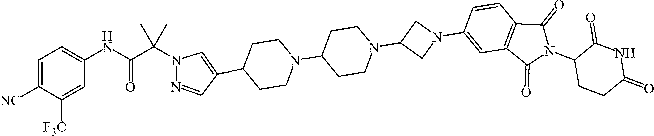 | 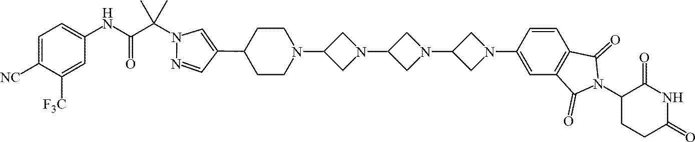 |
| 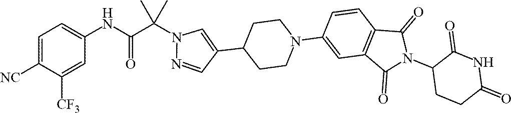 | 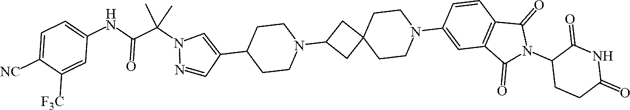 |
| 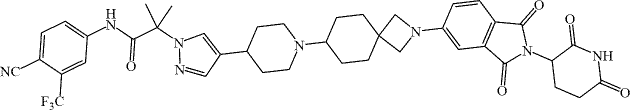 | 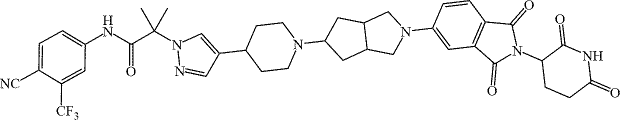 |
| 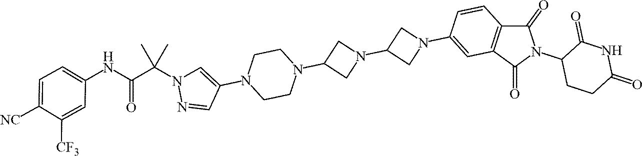 | 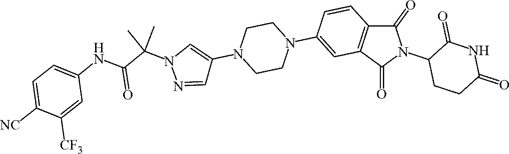 |
| 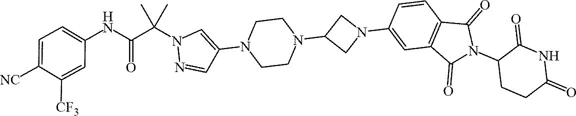 | 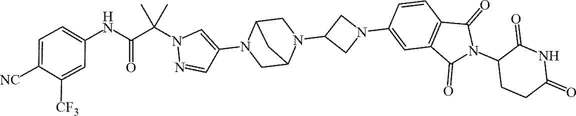 |
| 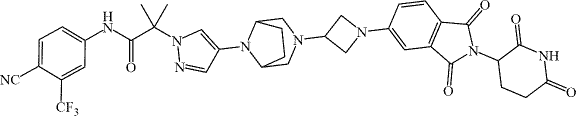 | 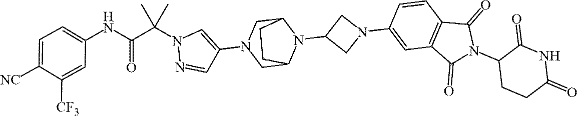 |
| 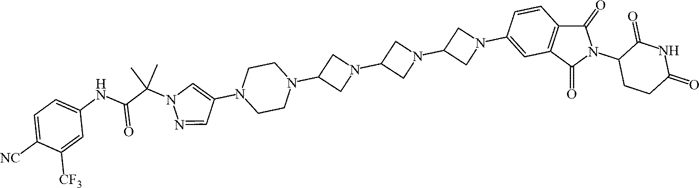 | 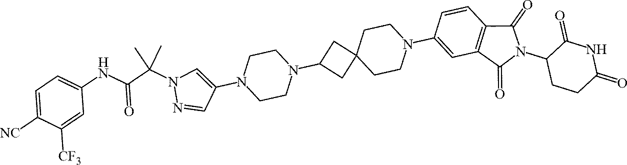 |
| 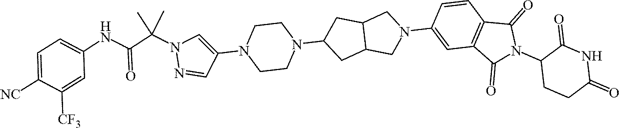 | 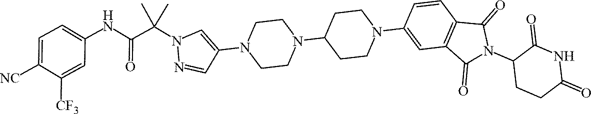 |
| 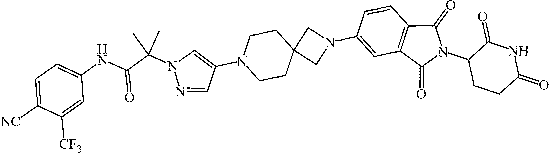 | 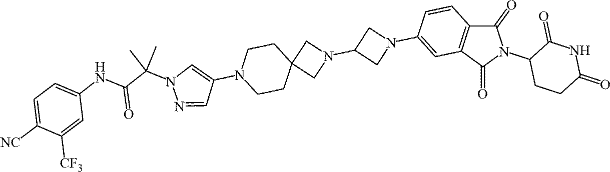 |
| 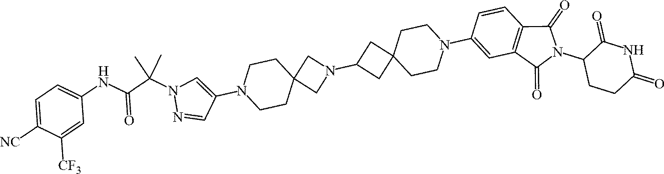 | 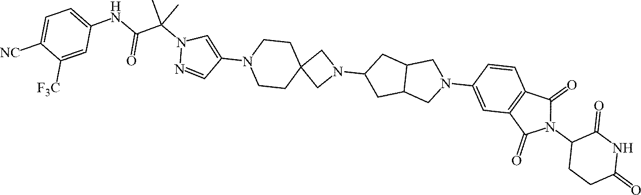 |
| 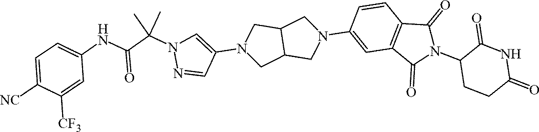 | 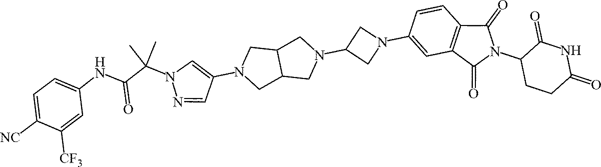 |
| 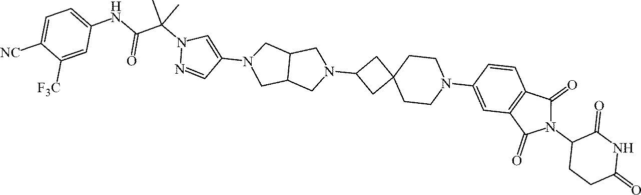 | 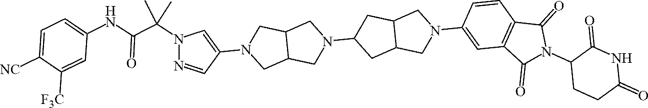 |
| 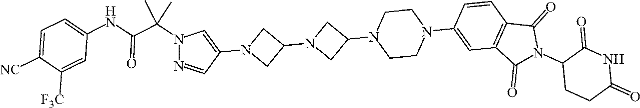 | 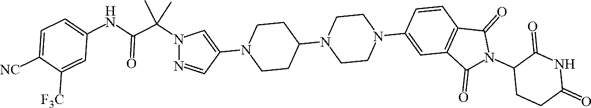 |
| 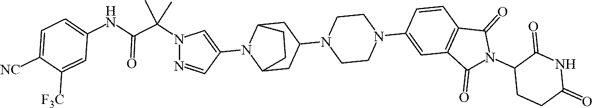 | 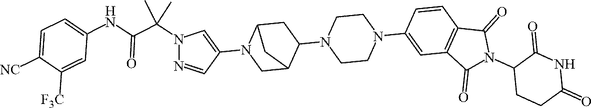 |
| 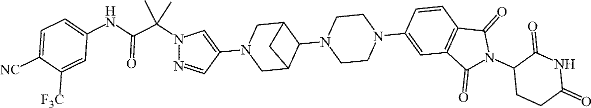 | 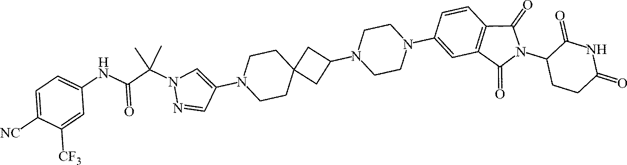 |
| 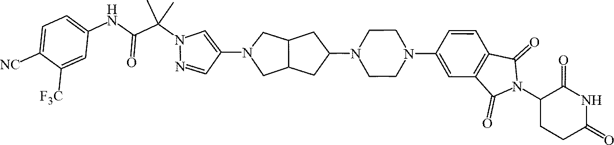 | 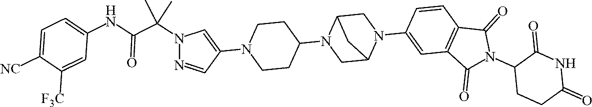 |
| 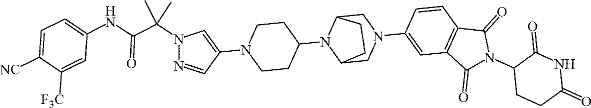 | 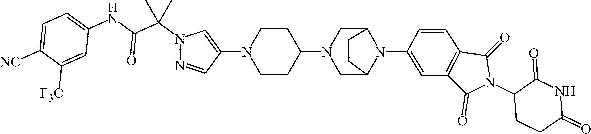 |
| 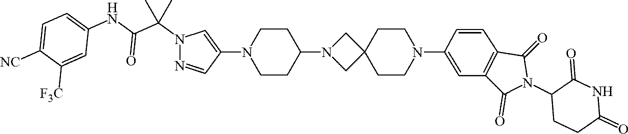 | 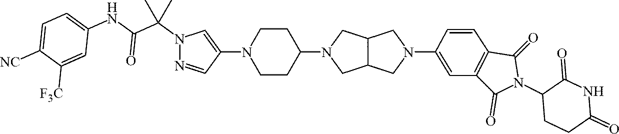 |
| 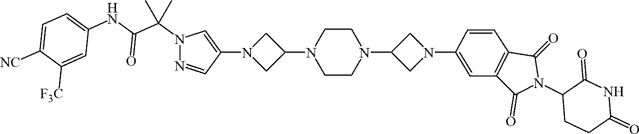 | 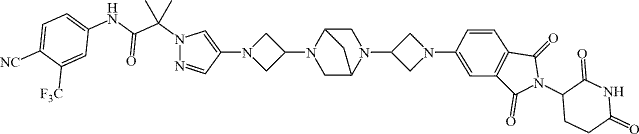 |
| 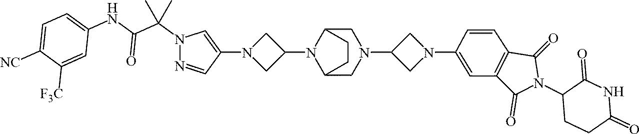 | 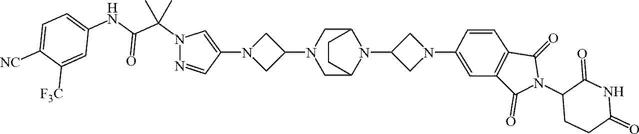 |
| 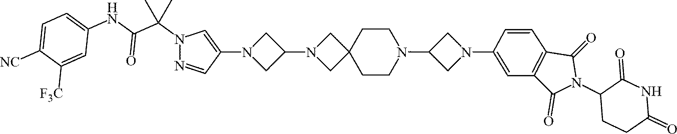 | 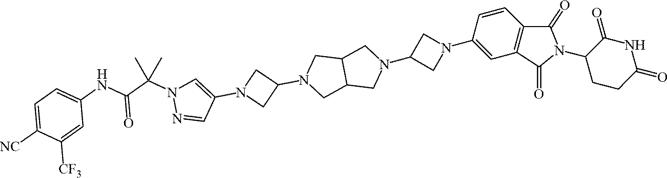 |
| 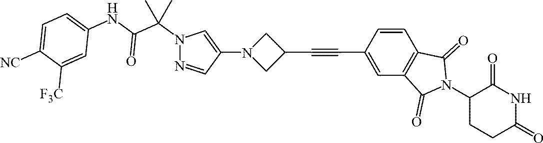 | 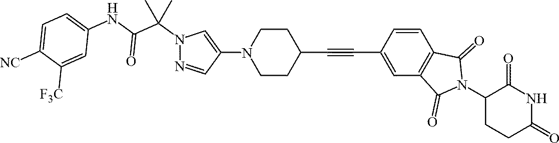 |
| 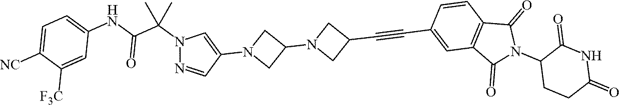 | 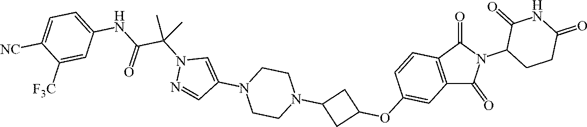 |
| 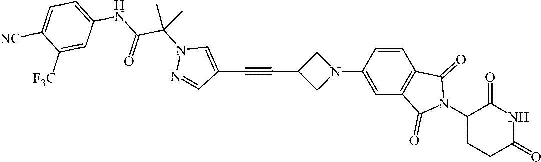 | 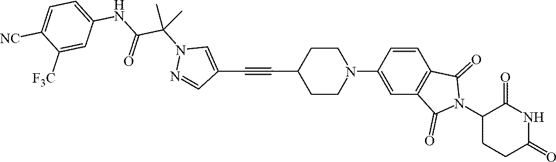 |
| 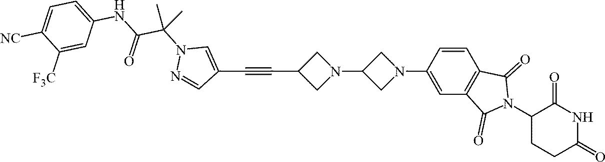 | 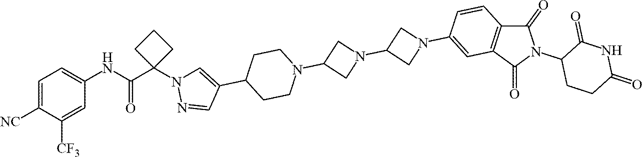 |
| 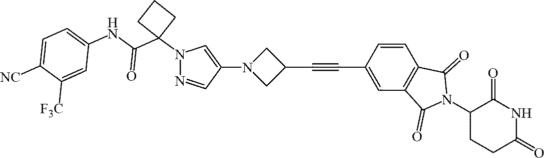 | 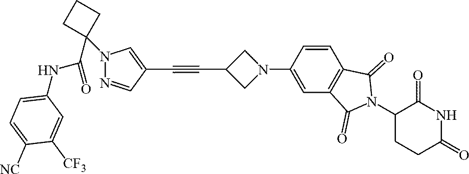 |
| 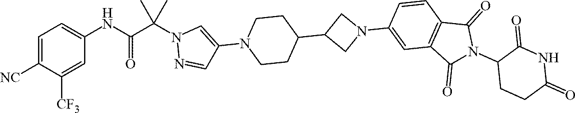 | 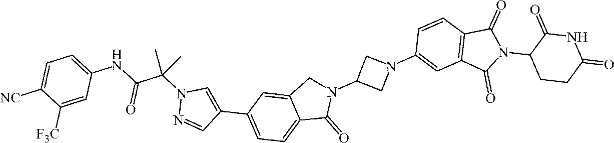 |
| 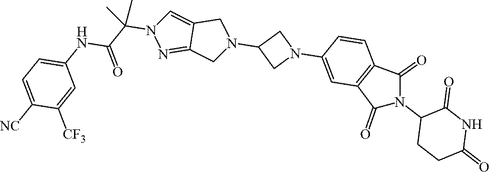 | 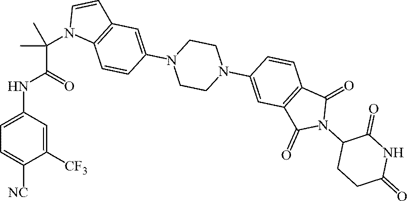 |
| 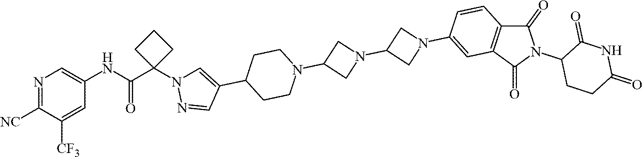 | 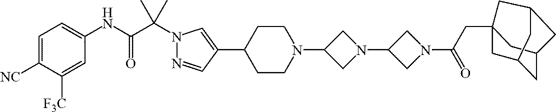 |
| 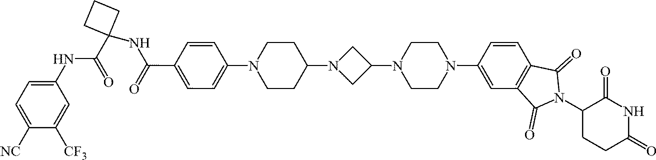 | 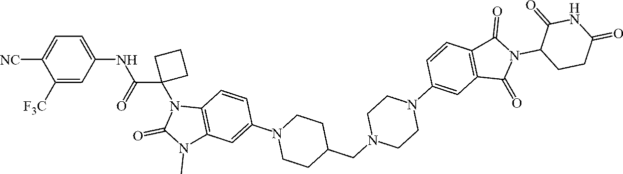 |
| 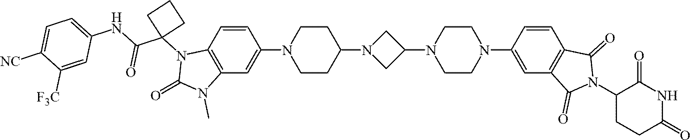 | 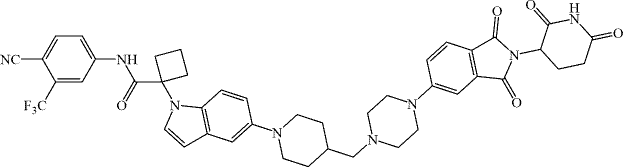 |
| 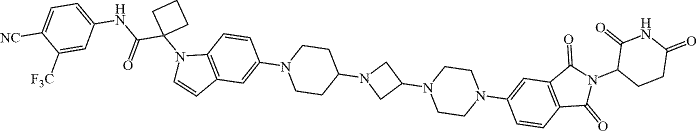 | 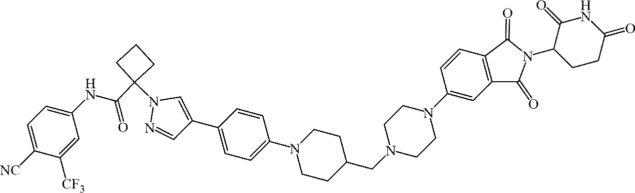 |
| 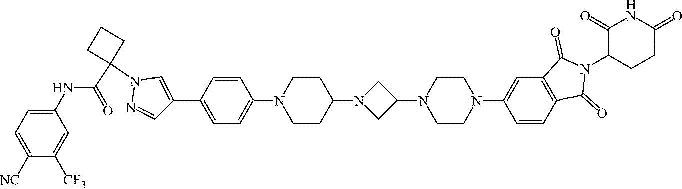 | 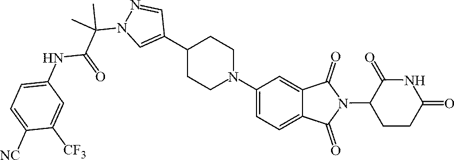 |
| 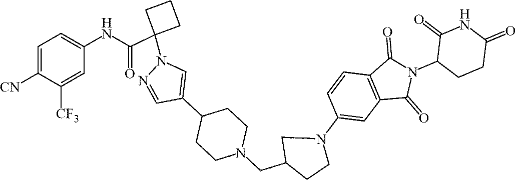 [SMILES: N#Cc1ccc(NC(=O)C2(n3cc(C4CCN(CC5CCN(c6ccc7c(c6)C(=O)N(C6CCC(=O)NC6=O)C7=O)C5)CC4)cn3)CCC2)cc1C(F)(F)F] | 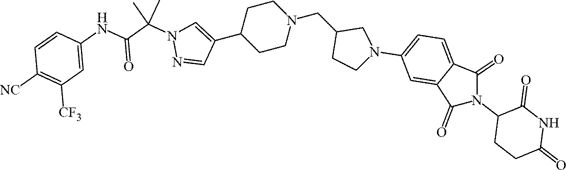 |
| 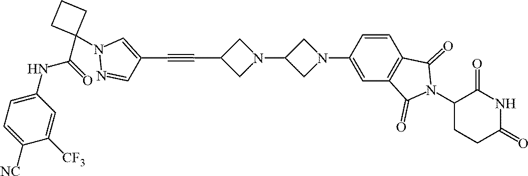 | 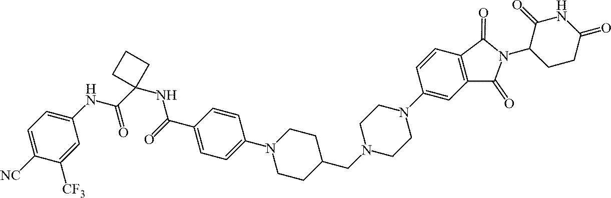 |

.

9. A pharmaceutical composition, comprising the compound or the stereoisomer, deuterated compound, solvate, prodrug, metabolite, pharmaceutically acceptable salt or co-crystal thereof according to claim 1, and a pharmaceutically acceptable carrier.

10. (canceled)

11. (canceled)

12. The method according to claim 13, wherein the disease is selected from prostate cancer.

13. A method for treating a disease related to AR activity or expression level or a disease related to the inhibition or degradation of AR, wherein the method comprises administering the compound or the stereoisomer, deuterated compound, solvate, prodrug, metabolite, pharmaceutically acceptable salt or co-crystal thereof according to claim 1.
